(19) United States
(12) Patent Application Publication
(10) Pub. No.: US 2021/0093637 A1
(43) Pub. Date: Apr. 1, 2021

(54) TOPICAL OINTMENT FORMULATIONS OF PDE-4 INHIBITOR AND THEIR USE IN TREATING SKIN CONDITIONS

(71) Applicant: Dermavant Sciences GmbH, Basel (CH)

(72) Inventors: James LEE, Durham, NC (US); Adam SIMPSON, Durham, NC (US)

(21) Appl. No.: 16/768,898

(22) PCT Filed: Dec. 7, 2018
(86) PCT No.: PCT/US2018/064580

§ 371 Date: Jun. 2, 2020

Related U.S. Application Data
(60) Provisional application No. 62/695,389, filed on Jul. 9, 2018.
(60) Provisional application No. 62/634,242, filed on Feb. 23, 2018.
(60) Provisional application No. 62/595,943, filed on Dec. 7, 2017.

(51) Int. Cl. A61K 31/517; A61K 9/00; A61K 47/22; A61K 47/10; A61K 47/12; A61K 47/06; A61K 47/34; A61P 17/00; A61P 17/04; A61K 9/06

(52) U.S. Cl. CPC A61K 31/517; A61K 9/0014; A61K 47/22; A61K 47/10; A61K 9/06; A61K 47/06; A61K 47/34; A61P 17/00; A61P 17/04; A61K 47/12

(57) ABSTRACT

Embodiments herein are directed to topical compositions comprising a therapeutically effective amount of methyl N-[3-(6,7-dimethoxy-2-methylaminoquinazolin-4-yl)phenyl]terephthalamic acid, PEG 400, PEG 4000, white petrolatum, vitamin E, glycerol monostearate/glycerides, isopropyl myristate, and water. The topical compositions may be used to treat a variety of skin conditions, including atopic dermatitis. Patients treated include pediatrics, adolescents and adults.

### CROSS-REFERENCE TO RELATED APPLICATIONS

This application claims the priority benefit under 35 U.S.C. 119(e) of U.S. Provisional Application No. 62/595,943 filed Dec. 7, 2017, U.S. Provisional Application No. 62/634,242 filed Feb. 23, 2018, and U.S. Provisional Application No. 62/695,389 filed Jul. 9, 2018 each disclosure of which is incorporated by reference in their entireties.

### SUMMARY

Embodiments herein are directed to topical compositions comprising a therapeutically effective amount of methyl N-[3-(6,7-dimethoxy-2-methylaminoquinazolin-4-yl)phenyl]terephthalamic acid, PEG 400, PEG 4000, white petrolatum, vitamin E, glycerol monostearate/glycerides, isopropyl myristate, and water.

Some embodiments herein are directed to methods of treating skin conditions in a patient in need thereof comprising topically applying a topical composition comprising a therapeutically effective amount of methyl N-[3-(6,7-dimethoxy-2-methylaminoquinazolin-4-yl)phenyl]terephthalamic acid, PEG 400, PEG 4000, white petrolatum, vitamin E, glycerol monostearate/glycerides, isopropyl myristate, and water. In certain embodiments, the patient is an adolescent. In certain embodiments, the skin condition is atopic dermatitis.

### DESCRIPTION OF DRAWINGS

For a fuller understanding of the nature and advantages of the present invention, reference should be had to the following detailed description taken in connection with the accompanying drawings, in which:

FIG. 1 illustrates the mean amount (μg) of a compound of formula (I) of embodiments herein collected from the stratum corneum of each donor 24 hours after application of the topical formulation of embodiments herein.

FIG. 2 illustrates the mean amount (μg) of a compound of formula (I) of embodiments herein collected from the epidermis for each donor 24 hours after application of the topical formulation of embodiments herein.

FIG. 3 illustrates the mean amount (μg) of a compound of formula (I) of embodiments herein collected from the dermis for each donor 24 hours after application of the topical formulation of embodiments herein.

FIG. 4 illustrates the timeline of the protocol used in Example 2.

FIG. 5 illustrates hematoxylin and eosin staining of normal skin versus skin with atopic dermatitis lesions. Note the epidermal hyperplasia, hyperkeratosis, ulceration, and immune cell infiltration in the DNCB-induced skin.

FIG. 6 illustrates hematoxylin and eosin staining of skin sections treated for atopic dermatitis skin lesions prophylactically (left) or therapeutically (right) at 40× magnification.

FIG. 7 illustrates select cytokine data from prophylactic (top) and therapeutic (bottom) studies. Featured cytokines are IL-6 (left), IL-17 (middle), and TNF-α (right). Data was collected from skin samples at day 15 in each study and run in a LUMINEX panel.

FIG. 8 illustrates scratching assay results in a prophylactic (top) and therapeutic (bottom) study.

FIG. 9 provides the response in IGA (0/1+2 point improvement) at week 4 in the ITT population.

FIG. 10 provides the response in IGA (0/1+2 point improvement) at week 4 in the PPS population.

FIG. 11 provides the response in IGA (0/1) at week 4 in the ITT population.

FIG. 12 provides the response in IGA (0/1) at week 4 in the PPS population.

FIG. 13 provides the IGA response (0/1+2 point improvement) kinetics in the ITT population.

FIG. 14 provides the IGA response (0/1+2 point improvement) kinetics in the PPS population.

FIG. 15 shows the EASI % improvement from baseline and the week 4 EASI % improvement in the ITT population.

FIG. 16 provides data of EASI 50/75/90 responders at week 4 for the ITT population.

FIG. 17 provides data of EASI 50/75/90 responders at week 4 for the PPS population.

FIG. 18 shows the improvement in NRS (itch) from baseline in the ITT population.

FIG. 19 shows the improvement in NRS (itch) from baseline at week 4 in the ITT population.

FIG. 20 shows the improvement in NRS (itch) from baseline at week 4 in the PPS population.

FIG. 21 shows the BSA % improvement from baseline and the week 4 BSA % improvement in the ITT population.

### DETAILED DESCRIPTION

This invention is not limited to the particular processes, compositions, or methodologies described, as these may vary. The terminology used in the description is for the purpose of describing the particular versions or embodiments only, and is not intended to limit the scope of the present invention. Unless defined otherwise, all technical and scientific terms used herein have the same meanings as commonly understood by one of ordinary skill in the art. All publications mentioned herein are incorporated by reference in their entirety. Nothing herein is to be construed as an admission that the invention is not entitled to antedate such disclosure by virtue of prior invention.

It must be noted that, as used herein, and in the appended claims, the singular forms “a,” “an,” and “the” include plural reference unless the context clearly dictates otherwise.

As used herein, the term “about” means plus or minus 10% of the numerical value of the number with which it is being used. Therefore, about 50% means in the range of 45% to 55%.

“Administering” when used in conjunction with a composition means to administer a composition to a patient whereby it positively impacts the tissue to which it is targeted, e.g. the skin. “Administering” a composition may be accomplished by, for example, topical administration, or in combination with other known techniques. Administering may be self-administration, wherein the subject in need of such treatment administers a composition or administering may be by a medical or other health care professional or a caretaker of the subject in need of such treatment.

The term “adolescent” as used herein is a human that is about 12 years of age to less than 17 years of age.

The term “patient” and “subject” are interchangeable and may be taken to mean any human which may be treated with compounds of the present invention. In some embodiments, the patient or subject is an adult, adolescent, child or infant. In some embodiments, the patient or subject is an adult, 18 years old or greater. In some embodiments, the patient or subject is an adolescent, ages 12-17 years old. In some embodiments, the patient or subject is a pediatric individual, ages 2-11 years old.

As used herein, the terms “comprising,” “comprise,” “comprises,” and “comprised” are inclusive or open-ended and do not exclude additional, unrecited elements or method steps.

As used herein, the term “consists of” or “consisting of” means that the composition or method includes only the elements, steps, or ingredients specifically recited in the particular embodiment or claim.

As used herein, the term “consisting essentially of” or “consists essentially of” means that the composition or method includes only the specified materials or steps and those that do not materially affect the basic and novel characteristics of the claimed invention.

Specific embodiments disclosed herein may be further limited in the claims using “consisting of” or “consisting essentially of” language, rather than “comprising”. In other words, though embodiments described herein use the phrase “comprising” or “comprises,” any embodiment described herein can be replaced with “consisting of”/“consists of” or “consisting essentially of”/“consists essentially of.”

The term “dermatitis” is used to refer to a group of skin conditions which result in inflammation of the skin and is characterized by itchiness, red skin and a rash. Included in this group are atopic dermatitis, contact dermatitis, allergic contact dermatitis, irritant contact dermatitis, stasis dermatitis, seborrheic dermatitis, chronic dermatitis, and eczema.

The term “therapeutically effective amount” refers to an amount of a composition, of the embodiments described herein, necessary or sufficient to achieve the desired effect. For example, in some embodiments, the desired effect may include, without limitation, medically therapeutic, cosmetically therapeutic and/or prophylactic treatment, as appropriate.

The terms “exfoliative keratolysis” or “keratolysis exfoliative” refer to a skin condition which is characterized by dry skin and superficial, air-filled blisters. These blisters can be peeled off very easily and will leave reddish, tender areas.

“Follicular hyperkeratinization” plays a key role in the pathogenesis of acne, cells of the follicle become cohesive and do not shed normally onto the skin's surface and results in a microcomedone.

The term “Geleol™” refers to glyceryl monostearate or glycerol monostearate/glycerides.

The term “ichthyosis” refers to a genetic skin disorder characterized by dry, thickened, and scaly skin.

In each of the embodiments disclosed herein, the compositions and methods may be utilized with or on a subject in need of such treatment, which may also be referred to as “in need thereof.” As used herein, the phrase “in need thereof” means that the subject has been identified as having a need for the particular method or treatment and that the treatment has been given to the subject for that particular purpose.

The terms “keratosis follicularis” or “Darier's disease” refer to a genetic disorder characterized by dark crusty patches on the skin, sometimes containing pus.

The term “lichen simplex chronicus” refers to a skin disorder characterized by chronic itching and scratching. The constant scratching causes thick, leathery, darkened, (lichenified) skin.

The term “lichen planus” refers to a disease characterized by itchy reddish-purple polygon-shaped skin lesions on the lower back, wrists, and ankles.

As used herein, the term “methyl N-[3-(6,7-dimethoxy-2-methylaminoquinazolin-4-yl)phenyl]terephthalamic acid,” “E6005,” or “RVT-501” shall also refer to alternative names of the compound, including N-[3-(6,7-dimethoxy-2-methylaminoquinazolin-4-yl)phenyl]terephthalamic methyl ester, methyl 4-[(3-[6,7-dimethoxy-2-(methylamino)quinazolin-4-yl]phenyl)carbamoyl]benzoate, and methyl 4-[({3-[6,7-dimethoxy-2-(methylamino)quinazolin-4-yl]phenyl}amino)carbonyl]benzoate. The compound represented as RVT-501 or E6005 is methyl N-[3-(6,7-dimethoxy-2-methylaminoquinazolin-4-yl)phenyl]terephthalamic acid having the structure:

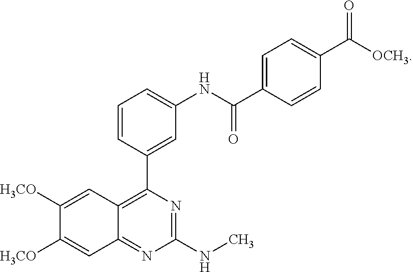 [SMILES: CNc1nc(-c2cccc(NC(=O)c3ccc(C(=O)OC)cc3)c2)c2cc(OC)c(OC)cc2n1]

As used herein, the term “pharmaceutically acceptable” and grammatical variations thereof, as they refer to carriers, diluents, excipients, and reagents or other ingredients of the composition, represent that the materials used in the final composition are not irritating or otherwise harmful to the patient in general and to the skin, in particular, and preferably are pleasant and well tolerated with respect to general appearance, pH, color, smell and texture (feel), that they are not, for example, unacceptably sticky (tacky), oily or drying, and that they do spread easily, absorb into the skin at an acceptable rate of absorption.

As used herein, the terms “metabolite of E6005,” “ER-392710,” or “M11” refer to the metabolite of methyl N-[3-(6,7-dimethoxy-2-methylaminoquinazolin-4-yl)phenyl]terephthalamic acid. The compound of M11 is 4-((3-(6,7-dimethoxy-2-(methylamino)quinazolin-4-yl)phenyl)carbamoyl)benzoic acid and has the structure:

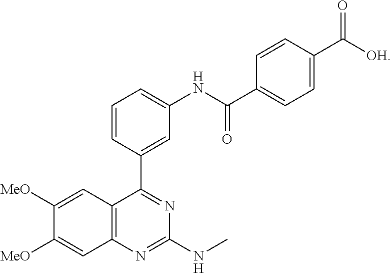 [SMILES: CNc1nc(-c2cccc(NC(=O)c3ccc(C(=O)O)cc3)c2)c2cc(OC)c(OC)cc2n1]

The term “Pityriasis rubra pilaris” refers to a group of chronic disorders characterized by reddish orange, scaling plaques and keratotic follicular papules. Symptoms may include reddish-orange patches on the skin, severe flaking, uncomfortable itching, thickening of the skin on the feet and hands, and thickened bumps around hair follicles.

The term “psoriasis” refers to the autoimmune disease characterized by patches of abnormal skin which is red, itchy and scaly. There are five main types of psoriasis: plaque, guttate, inverse, pustular, and erythrodermic.

The terms “pruritus” or “prurigo” refer to the severe itching of the skin due to a variety of ailments.

The term “palmoplantar pustulosis” refers to a chronic pustular condition affecting the palms and soles.

The term “rosacea” refers to a skin condition characterized by redness, pimples, swelling, and small, superficial dilated blood vessels.

The term “sebaceous adenomas” refers to a small bump on the skin, when many small bumps appear it is referred to as “sebaceous hyperplasia.”

The term “sebaceous gland” includes unilobular or multilobular glands that secrete sebum. Sebaceous glands include pilosebaceous units, fordyce spots, Meibomian glands, glands of the Zeiss and Montgomery areolar tubercles.

The phrase “disorder associated with sebaceous glands” includes diseases, conditions and symptoms related to sebaceous gland. Disorders associated with sebaceous glands include acne, seborrhea, sebaceoma, sebaceous carcinoma, seborrheic dermatitis, sebaceous cysts, sebaceous adenoma and sebaceous hyperplasia.

The term “seborrhea” includes oily skin.

The term “seborrheic dermatitis” includes inflammatory skin disorders characterized by scaly, flaky, itchy, and red skin and includes seborrheic dermatitis caused by fungal, genetic, environmental, hormonal and immune function disorders.

The term “sebaceous cysts” include steatocystoma simplex (e.g., simple sebaceous duct cyst and solitary steatocystoma) and steatocystoma multiplex (e.g., epidermal polycystic disease and sebocystomatosis).

The term “sebaceous hyperplasia” includes enlargement of the sebaceous glands.

The term “skin” as used herein refers to the organ of the body which protects the subject from environmental irritations, regulates the body's temperature and allows for external sensations. The “skin” is separated into three layers: the outermost layer called the epidermis which contains melanocytes; the dermis which contains connective tissue, hair follicles and sweat glands; and the deepest subcutaneous layer called the hypodermis which is made up of fat and connective tissue.

As used herein, the term “topically” and “topical” refers to application of the compositions of the present invention to the surface of the skin and mucous membranes.

“Topical application” or “topical administration” refers to the delivery of a composition, for treating conditions of the epidermis or dermis, wherein the topical composition is applied to the skin and acts locally and does not have a systemic effect. Topical administration of a drug may often be advantageously applied in, for example, the treatment of various skin disorders.

As used herein the terms “topical formulations” and “topical compositions” refer to formulations or compositions that may be applied to skin or mucous membranes. Topical formulations or compositions may, for example, be used to confer therapeutic benefit to a patient or cosmetic benefit to a consumer. Such topical formulations or compositions may be provided in the form of a cream, foam, gel, lotion, or ointment.

The terms “treat,” “treated,” or “treating” as used herein refers to therapeutic treatment, cosmetic treatment and/or prophylactic or preventative measures, wherein the object is to prevent, reduce, eliminate or slow down (lessen) an undesired physiological condition, disorder or disease, or to obtain beneficial or desired clinical results (e.g. decrease acne, comedones, pimples, or breakouts). For the purposes of this disclosure, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of unwanted side effects.

The term “wart” refers to the small, rough, and hard growths that are similar in color to the rest of the skin caused by caused by infection with a type of human papillomavirus (HPV). A number of types exist including: common warts, plantar warts, filiform warts, and genital warts.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability.

### Compound of Formula (I)

In some embodiments, the compound represented by the formula (I) is methyl N-[3-(6,7-dimethoxy-2-methylaminoquinazolin-4-yl)phenyl]terephthalamic acid (RVT-501) having the structure:

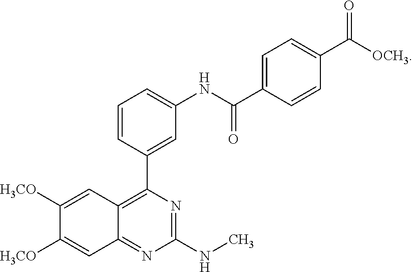 [SMILES: CNc1nc(-c2cccc(NC(=O)c3ccc(C(=O)OC)cc3)c2)c2cc(OC)c(OC)cc2n1]

The compound and methods of making such compound are further described in U.S. Pat. Nos. 7,939,540 and 8,530,654, which are each hereby incorporated by reference in its entirety.

Optical Isomers-Diastereomers-Geometric Isomers-Tautomers. Compounds described herein may contain an asymmetric center and may thus exist as enantiomers. Where the compounds according to the invention possess two or more asymmetric centers, they may additionally exist as diastereomers. Embodiments herein include all such possible stereoisomers as substantially pure resolved enantiomers, racemic mixtures thereof, as well as mixtures of diastereomers. The formulas are shown without a definitive stereochemistry at certain positions. Embodiments herein include all stereoisomers of such formulas and pharmaceutically acceptable salts thereof. Diastereoisomeric pairs of enantiomers may be separated by, for example, fractional crystallization from a suitable solvent, and the pair of enantiomers thus obtained may be separated into individual stereoisomers by conventional means, for example by the use of an optically active acid or base as a resolving agent or on a chiral HPLC column. Further, any enantiomer or diastereomer of a compound of the general formula may be obtained by stereospecific synthesis using optically pure starting materials or reagents of known configuration. Embodiments described herein include all isomers of the compound of formula (I) disclosed herein, such as a geometric isomer, an optical isomer, a stereoisomer, or a tautomer, and an isomeric mixture. Embodiments herein include both the racemic form and the optically active form. Embodiments further include a single crystal form or a mixture thereof. Moreover, embodiments herein also include an amorphous form, an anhydrate, and a hydrate form of the compound. Furthermore, embodiments herein also include metabolites, salts, hydrates, and pro-drugs of the compounds disclosed herein.

In some embodiments, a salt of compounds described herein may include an inorganic acid salt, an organic acid salt, an inorganic basic salt, an organic basic salt, an acidic or basic amino acid salt or the like. In some embodiments, the inorganic acid salt may include hydrochloride, hydrobromide, sulfate, nitrate, phosphate or the like. In some embodiments, the salt may be selected from a hydrochloride, hydrobromide, sulfate, or phosphate. In some embodiments, the organic acid salt may include acetate, succinate, fumarate, maleate, tartrate, citrate, lactate, stearate, benzoate, methanesulfonate, ethanesulfonate, p-toluenesulfonate, or benzenesulfonate. In some embodiments, the salt may be methanesulfonate or p-toluenesulfonate.

In some embodiments, the inorganic basic salt may include: alkaline metal salts such as a sodium salt or a potassium salt; alkaline-earth metal salts such as a calcium salt or a magnesium salt; aluminum salts; ammonium salts, or the like. In some embodiments, the organic basic salt may include a diethylamine salt, a diethanolamine salt, a meglumine salt, an N,N′-dibenzylethylenediamine salt, or the like.

In some embodiments, the acidic amino acid salt may include aspartate and glutamate. In some embodiments, the basic amino acid salt may include an arginine salt, a lysine salt, an ornithine salt or the like.

### Topical Formulations

In some embodiments, the active ingredient is methyl N-[3-(6,7-dimethoxy-2-methylaminoquinazolin-4-yl)phenyl]terephthalamic acid (RVT-501):

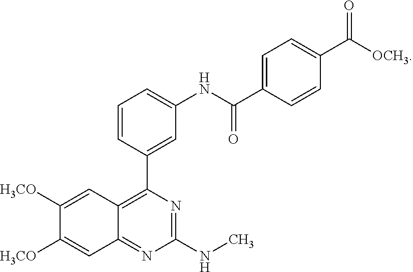 [SMILES: CNc1nc(-c2cccc(NC(=O)c3ccc(C(=O)OC)cc3)c2)c2cc(OC)c(OC)cc2n1]

Embodiments herein are directed to topical compositions comprising a therapeutically effective amount of methyl N-[3-(6,7-dimethoxy-2-methylaminoquinazolin-4-yl)phenyl]terephthalamic acid, PEG 400, PEG 4000, white petrolatum, vitamin E, glycerol monostearate/glycerides, isopropyl myristate, and water. In some embodiments, methyl N-[3-(6,7-dimethoxy-2-methylaminoquinazolin-4-yl)phenyl]terephthalamic acid is at a concentration of about 0.01% to about 5% by weight of the topical composition. In some embodiments, PEG 400 is at a concentration of about 25% to about 75% by weight of the topical composition. In some embodiments, PEG 4000 is at a concentration of about 15% to about 35% by weight of the topical composition. In some embodiments, white petrolatum is at a concentration of about 1% to about 10% by weight of the topical composition. In some embodiments, vitamin E is at a concentration of about 0.01% to about 5% by weight of the topical composition. In some embodiments, glycerol monostearate/glycerides is at a concentration of about 2% to about 15% by weight of the topical composition. In some embodiments, isopropyl myristate is at a concentration of about 2% to about 25% by weight of the topical composition. In some embodiments, water is at a concentration of about 0.1% to about 10% by weight of the topical composition.

In certain embodiments, a topical composition comprises methyl N-[3-(6,7-dimethoxy-2-methylaminoquinazolin-4-yl)phenyl]terephthalamic acid at 0.2% by weight, PEG 400 at 50.5% by weight, PEG 4000 at 25.0% by weight, white petrolatum at 4.4% by weight, vitamin E at 0.1% by weight, glycerol monostearate/glycerides at 8.0% by weight, isopropyl myristate at 10.0% by weight, and water at 2.0% by weight.

In certain embodiments, a topical composition comprises methyl N-[3-(6,7-dimethoxy-2-methylaminoquinazolin-4-yl)phenyl]terephthalamic acid at 0.5% by weight, PEG 400 at 50.5% by weight, PEG 4000 at 25.0% by weight, white petrolatum at 4.4% by weight, vitamin E at 0.1% by weight, glycerol monostearate/glycerides at 8.0% by weight, isopropyl myristate at 10.0% by weight, and water at 2.0% by weight.

Embodiments herein are directed to a topical composition comprising a therapeutically effective amount of methyl N-[3-(6,7-dimethoxy-2-methylaminoquinazolin-4-yl)phenyl]terephthalamic acid and pharmaceutically acceptable topical excipients, wherein 90% confidence interval for the ratio of means (population geometric means based on log-transformed data) of the AUC of the topical composition is within 80-125% of the AUC of any one the foregoing topical compositions and the 90% confidence internal for the ratio of means of the Cₘₐₓ of the topical composition is within 70-143% of the Cₘₐₓ of the same foregoing topical composition.

The topical compositions of the present invention may be formulated by those skilled in the art as liquids, toners, solutions, sprays, emulsions, moisturizers, sunscreens, creams, lotions, masks, suspensions, triturates, gels, jellies, pastes, foams, ointments, shampoos, adhesives, serums, treated clothes or pads and the like. In some embodiments the topical composition is formulated as eye drops, as ear drops, or as a composition which can be applied to the surface of the tooth.

In embodiments described herein, the topical compositions may be applied to the skin by any means known in the art including, but not limited to, by an aerosol, spray, pump-pack, brush, swab, or other applicator. The applicator may provide either a fixed or variable metered dose application such as a metered dose aerosol, a stored-energy metered dose pump or a manual metered dose pump.

In embodiments described herein, the topical composition is formulated as to be applied to a site one time a day or multiple times per day.

### Methods of Using the Topical Formulations

Embodiments described herein are directed to methods of treating mild to moderate atopic dermatitis in a patient in need thereof comprising topically applying a topical composition comprising a therapeutically effective amount of methyl N-[3-(6,7-dimethoxy-2-methylaminoquinazolin-4-yl)phenyl]terephthalamic acid, PEG 400, PEG 4000, white petrolatum, vitamin E, glycerol monostearate/glycerides, isopropyl myristate, and water. Embodiments described herein the patient may of different patient populations, wherein the patient maybe a pediatric, an adolescent, or an adult. In embodiments described herein, the therapeutically effective amount of methyl N-[3-(6,7-dimethoxy-2-methylaminoquinazolin-4-yl)phenyl]terephthalamic acid is 0.2% or 0.5%. In embodiments described herein, the topical composition is applied once per day or twice per day. Embodiments described herein are directed to methods of treating mild to moderate atopic dermatitis in a patient in need thereof in accordance with Example 2: Treatment of Atopic Dermatitis, Example 3: Phase 2 Study of RVT-501 in Adult and Adolescent Subjects with Atopic Dermatitis, Example 6: Phase 2 Study to Evaluate the Efficacy, Safety, and Tolerability of RVT-501 Topical Ointment in Pediatric Patients With Mild to Moderate Atopic Dermatitis, or Example 7: Open-Label Study to Evaluate the Safety, Tolerability, and Pharmacokinetics of RVT-501 Topical Ointment in Pediatric Patients With Atopic Dermatitis.

Embodiments herein are directed to methods of treating a skin condition in a patient in need thereof comprising topically applying a topical composition comprising a therapeutically effective amount of methyl N-[3-(6,7-dimethoxy-2-methylaminoquinazolin-4-yl)phenyl]terephthalamic acid, PEG 400, PEG 4000, white petrolatum, vitamin E, glycerol monostearate/glycerides, isopropyl myristate, and water. In certain embodiments, the patient is an adolescent.

In some embodiments, methyl N-[3-(6,7-dimethoxy-2-methylaminoquinazolin-4-yl)phenyl]terephthalamic acid is at a concentration of about 0.01% to about 5% by weight of the topical composition. In some embodiments, PEG 400 is at a concentration of about 25% to about 75% by weight of the topical composition. In some embodiments, PEG 4000 is at a concentration of about 15% to about 35% by weight of the topical composition. In some embodiments, white petrolatum is at a concentration of about 1% to about 10% by weight of the topical composition. In some embodiments, vitamin E is at a concentration of about 0.01% to about 5% by weight of the topical composition. In some embodiments, glycerol monostearate/glycerides is at a concentration of about 2% to about 15% by weight of the topical composition. In some embodiments, isopropyl myristate is at a concentration of about 2% to about 25% by weight of the topical composition. In some embodiments, water is at a concentration of about 0.1% to about 10% by weight of the topical composition.

In certain embodiments, the method of treating a skin condition in a patient in need thereof comprises topically applying a topical composition comprising methyl N-[3-(6,7-dimethoxy-2-methylaminoquinazolin-4-yl)phenyl]terephthalamic acid at 0.2% by weight, PEG 400 at 50.5% by weight, PEG 4000 at 25.0% by weight, white petrolatum at 4.4% by weight, vitamin E at 0.1% by weight, glycerol monostearate/glycerides at 8.0% by weight, isopropyl myristate at 10.0% by weight, and water at 2.0% by weight.

In certain embodiments, the method of treating a skin condition in a patient in need thereof comprises topically applying a topical composition comprising methyl N-[3-(6,7-dimethoxy-2-methylaminoquinazolin-4-yl)phenyl]terephthalamic acid at 0.5% by weight, PEG 400 at 50.5% by weight, PEG 4000 at 25.0% by weight, white petrolatum at 4.4% by weight, vitamin E at 0.1% by weight, glycerol monostearate/glycerides at 8.0% by weight, isopropyl myristate at 10.0% by weight, and water at 2.0% by weight.

Embodiments herein are directed to a topical composition comprising a therapeutically effective amount of methyl N-[3-(6,7-dimethoxy-2-methylaminoquinazolin-4-yl)phenyl]terephthalamic acid and pharmaceutically acceptable topical excipients, wherein 90% confidence interval for the ratio of means (population geometric means based on log-transformed data) of the AUC of the topical composition is within 80-125% of the AUC of any one the foregoing topical compositions and the 90% confidence internal for the ratio of means of the Cₘₐₓ of the topical composition is within 70-143% of the Cₘₐₓ of the same foregoing topical composition.

In certain embodiments, the skin condition being treated in a patient in need thereof is selected from the group consisting of dermatitis; psoriasis; itchy skin; acne; inflammation and redness of the skin; disorders associated with sebaceous glands; oily skin; dry skin; rosacea; burns; disorders affecting the palms or soles; genetic disorders of the skin; warts; and any combination thereof. In some embodiments, dermatitis is selected from the group consisting of atopic dermatitis, contact dermatitis, allergic contact dermatitis, irritant contact dermatitis, stasis dermatitis, seborrheic dermatitis, chronic dermatitis, eczema, and any combination thereof. In some embodiments, psoriasis is selected from the group consisting of plaque psoriasis, guttate psoriasis, inverse psoriasis, pustular psoriasis, erythrodermic psoriasis, and any combination thereof. In some embodiments, itchy skin is selected from the group consisting of pruritus, prurigo, Pityriasis rubra pilaris, lichen simplex chronicus, lichen planus, and any combination thereof. In some embodiments, acne is selected from the group consisting of acne vulgaris, cystic acne, inflammatory acne, non-inflammatory acne, and any combination thereof. In some embodiments, inflammation and redness of the skin is selected from the group consisting of seborrheic dermatitis, urticaria eczema, hives, seborrheic eczema, and any combination thereof. In some embodiments, disorders associated with sebaceous glands is selected from the group consisting of acne, follicular hyperkeratinization, sebostasis, sebaceous adenomas, sebaceous hyperplasia, excess sebum production, seborrhea, sebaceoma, sebaceous carcinoma, seborrheic dermatitis, sebaceous cysts, and any combination thereof. In some embodiments, oily skin is seborrhea. In some embodiments, dry skin is selected from the group consisting of sebostasis, ichthyosis, xerosis, and any combination thereof. In some embodiments, burns is sunburn. In some embodiments, disorders affecting the palms or soles is selected from the group consisting of palmoplantar pustulosis, exfoliative keratolysis, and any combination thereof. In some embodiments, genetic disorders of the skin is Darier's disease.

In some embodiments, the method of treating atopic dermatitis in a patient in need thereof comprises topically applying a topical composition comprising a therapeutically effective amount of methyl N-[3-(6,7-dimethoxy-2-methylaminoquinazolin-4-yl)phenyl]terephthalamic acid, PEG 400, PEG 4000, white petrolatum, vitamin E, glycerol monostearate/glycerides, isopropyl myristate, and water. In certain embodiments, the patient is an adolescent.

In some embodiments, methyl N-[3-(6,7-dimethoxy-2-methylaminoquinazolin-4-yl)phenyl]terephthalamic acid is at a concentration of about 0.01% to about 5% by weight of the topical composition. In some embodiments, PEG 400 is at a concentration of about 25% to about 75% by weight of the topical composition. In some embodiments, PEG 4000 is at a concentration of about 15% to about 35% by weight of the topical composition. In some embodiments, white petrolatum is at a concentration of about 1% to about 10% by weight of the topical composition. In some embodiments, vitamin E is at a concentration of about 0.01% to about 5% by weight of the topical composition. In some embodiments, glycerol monostearate/glycerides is at a concentration of about 2% to about 15% by weight of the topical composition. In some embodiments, isopropyl myristate is at a concentration of about 2% to about 25% by weight of the topical composition. In some embodiments, water is at a concentration of about 0.1% to about 10% by weight of the topical composition.

In certain embodiments, a method of treating atopic dermatitis in a patient in need thereof comprises topically applying a topical composition comprising methyl N-[3-(6,7-dimethoxy-2-methylaminoquinazolin-4-yl)phenyl]terephthalamic acid at 0.2% by weight, PEG 400 at 50.5% by weight, PEG 4000 at 25.0% by weight, white petrolatum at 4.4% by weight, vitamin E at 0.1% by weight, glycerol monostearate/glycerides at 8.0% by weight, isopropyl myristate at 10.0% by weight, and water at 2.0% by weight.

In certain embodiments, a method of treating atopic dermatitis in a patient in need thereof comprises topically applying a topical composition comprising methyl N-[3-(6,7-dimethoxy-2-methylaminoquinazolin-4-yl)phenyl]terephthalamic acid at 0.5% by weight, PEG 400 at 50.5% by weight, PEG 4000 at 25.0% by weight, white petrolatum at 4.4% by weight, vitamin E at 0.1% by weight, glycerol monostearate/glycerides at 8.0% by weight, isopropyl myristate at 10.0% by weight, and water at 2.0% by weight.

In embodiments described herein, the method is directed to applying a topical composition once per day. In embodiments described herein, the method is directed to applying a topical composition multiple times per day. In some embodiments, the topical composition is applied two times per day, three times per day, four times per day, or five times per day. In some embodiments, the topical composition is applied one time in the morning and one time in the evening. In some embodiments, the topical composition is applied every 12 hours, every 11 hours, every 10 hours, every 9 hours, every 8 hours, every 7 hours, every 6 hours, every 5 hours, every 4 hours, every 3 hours, every 2 hours, or every hour.

In embodiments described herein, the method is directed to applying a topical composition to multiple sites on the skin of the body. For example, the topical composition may be applied over large areas of skin prophylactically or the topical composition may be applied to particular sites in need of treatment. In some embodiments, the topical composition is applied to the skin as a liquid, toner, solution, spray, emulsion, moisturizer, sunscreen, cream, lotion, mask, suspension, triturate, gel, jelly, paste, foam, ointment, shampoo, adhesive, serum, treated cloth or pad. In some embodiments, the topical composition is applied to the eyes as eye drops, placed in the ear canal as ear drops or to the surface of the tooth.

### Methods of Detecting Serum Levels of Methyl N-[3-(6,7-dimethoxy-2-methylaminoquinazolin-4-yl)phenyl]terephthalamic Acid and its Metabolite

Embodiments herein are directed to methods of treating a condition in a patient comprising administering a topical composition comprising methyl N-[3-(6,7-dimethoxy-2-methylaminoquinazolin-4-yl)phenyl]terephthalamic acid, and analyzing the level of methyl N-[3-(6,7-dimethoxy-2-methylaminoquinazolin-4-yl)phenyl]terephthalamic acid and a metabolite in the patient's blood. In embodiments, the metabolite is 4-((3-(6,7-dimethoxy-2-(methylamino)quinazolin-4-yl)phenyl)carbamoyl)benzoic acid.

Embodiments herein are directed to methods of treating a condition in a child comprising administering a topical composition comprising methyl N-[3-(6,7-dimethoxy-2-methylaminoquinazolin-4-yl)phenyl]terephthalamic acid, and analyzing the level of methyl N-[3-(6,7-dimethoxy-2-methylaminoquinazolin-4-yl)phenyl]terephthalamic acid and a metabolite in the child's blood. In embodiments, the metabolite is 4-((3-(6,7-dimethoxy-2-(methylamino)quinazolin-4-yl)phenyl)carbamoyl)benzoic acid.

In embodiments, the child is less than 18 years old, less than 15 years old, less than 12 years old, less than 10 years old, less than 5 years old, less than 3 years old, less than 2 years old, or less than 1 year old. In embodiments, the child is an infant. In embodiments, the child weighs less than 50 pounds, less than 40 pounds, less than 30 pounds, less than 20 pounds, or less than 10 pounds.

Embodiments herein are directed to methods of monitoring levels of a drug and a metabolite in a patient's blood during treatment comprising administering a topical composition of the drug, collecting the patient's blood, and analyzing the level of the drug and a metabolite in the blood. In embodiments, the drug is methyl N-[3-(6,7-dimethoxy-2-methylaminoquinazolin-4-yl)phenyl]terephthalamic acid. In embodiments, the metabolite is 4-((3-(6,7-dimethoxy-2-(methylamino)quinazolin-4-yl)phenyl)carbamoyl)benzoic acid.

In embodiments, the level of drug and/or metabolite in the child or patient's blood may determine a treatment recommendation, wherein a level of drug and/or metabolite in the patient's blood is within an acceptable limit may result in the recommendation to continue drug treatment, whereas a level of drug and/or metabolite in the patient's blood outside of an acceptable limit may result in the discontinuation of the drug treatment or a change in the amount of drug treatment applied.

Embodiments herein are directed to methods of treating a skin condition in a patient in need thereof comprising: a) topically applying a topical composition comprising a therapeutically effective amount of methyl N-[3-(6,7-dimethoxy-2-methylaminoquinazolin-4-yl)phenyl]terephthalamic acid, b) collecting about 10 μL to about 1 mL of a blood sample from the patient, c) spotting the blood sample onto a dried blood spot card, and d) analyzing the blood sample for a level of methyl N-[3-(6,7-dimethoxy-2-methylaminoquinazolin-4-yl)phenyl]terephthalamic acid and 4-((3-(6,7-dimethoxy-2-(methylamino)quinazolin-4-yl)phenyl)carbamoyl)benzoic acid.

In embodiments, the patient is an infant or a child, and the volume of blood collected is about 1 mL, about 500 μL, about 100 μL, about 50 μL, about 40 μL, about 30 μL, about 25 μL, about 20 μL, about 15 μL, or about 10 μL.

Embodiments herein are directed to methods of detecting methyl N-[3-(6,7-dimethoxy-2-methylaminoquinazolin-4-yl)phenyl]terephthalamic acid and 4-((3-(6,7-dimethoxy-2-(methylamino)quinazolin-4-yl)phenyl)carbamoyl)benzoic acid comprising: a) collecting about 10 μL to about 1 mL of a blood sample from a patient, b) spotting the blood sample onto a dried blood spot card, c) punching a about 3 mm to about 10 mm disc out of the dried blood spot card and processing the blood sample, d) analyzing the processed blood sample using UPLC-MS/MS (Ultra Performance Liquid Chromatography-tandem Mass Spectrometry), and e) quantifying an amount of methyl N-[3-(6,7-dimethoxy-2-methylaminoquinazolin-4-yl)phenyl]terephthalamic acid and 4-((3-(6,7-dimethoxy-2-(methylamino)quinazolin-4-yl)phenyl)carbamoyl)benzoic acid in the blood sample.

In embodiments, the volume of blood collected is about 1 mL, about 500 μL, about 100 μL, about 50 μL, about 40 μL, about 30 μL, about 25 μL, about 20 μL, about 15 μL, or about 10 μL.

In embodiments, the disc punched out from the dried blood spot card is about 3 mm, about 4 mm, about 5 mm, about 6 mm, about 7 mm, about 8 mm, about 9 mm, or about 10 mm.

In embodiments, the amount of methyl N-[3-(6,7-dimethoxy-2-methylaminoquinazolin-4-yl)phenyl]terephthalamic acid quantified from the blood sample is from about 1 mg/mL to about 200 ng/mL. In embodiments, the amount of methyl N-[3-(6,7-dimethoxy-2-methylaminoquinazolin-4-yl)phenyl]terephthalamic acid quantified from the blood sample is 3 ng/mL. In embodiments, the amount of methyl N-[3-(6,7-dimethoxy-2-methylaminoquinazolin-4-yl)phenyl]terephthalamic acid quantified from the blood sample is 160 ng/mL.

In embodiments, the amount of 4-((3-(6,7-dimethoxy-2-(methylamino)quinazolin-4-yl)phenyl)carbamoyl)benzoic acid quantified from the blood sample is from about 1 mg/mL to about 200 ng/mL. In embodiments, the amount of 4-((3-(6,7-dimethoxy-2-(methylamino)quinazolin-4-yl)phenyl)carbamoyl)benzoic acid quantified from the blood sample is 3 ng/mL. In embodiments, the amount of 4-((3-(6,7-dimethoxy-2-(methylamino)quinazolin-4-yl)phenyl)carbamoyl)benzoic acid quantified from the blood sample is 160 ng/mL.

### EXAMPLES

### Example 1: Skin Penetration Study

The study was designed to evaluate the penetration of an active ingredient, methyl N-[3-(6,7-dimethoxy-2-methylaminoquinazolin-4-yl)phenyl]terephthalamic acid (RVT-501), into and across human cadaver skin from 4 formulations and 1 drug solution using the in vitro Franz finite dose model with human cadaver skin. Phosphate buffered saline; pH 7.4±0.1 was used as receiving medium. Each cell was dosed once with 10 μL/cm2 of the respective formulation using a positive displacement pipette. At pre-selected times after dose application, a 500 μL aliquot of receiving media was removed through the sampling arm of the Franz cell and replaced with an equal volume of fresh receiving medium. A glass rod was used to spread the formulation evenly covering the entire surface area of the skin. At the conclusion of the study, the cells were disassembled and the skin was carefully removed from each cell. Each skin section was washed twice with 0.5 mL of extraction solution (the receiving medium) to collect un-absorbed formulation from the surface of the skin. The skin was carefully separated into epidermis and dermis using forceps. To each epidermis and dermis vial, homogenization solution (phosphate buffered saline, pH 7.4) was added. Tissues were homogenized using a bead homogenizer (OMNI Bead Ruptor 24.)

TABLE 1
Formulations

| | Formulations | | | | |
|---|---|---|---|---|---|
| | B | C1 | C2 | C3 | C4 |
| | Strength (%) | | | | |
| Ingredients | 0.2 | 0.2 | 0.5 | 0.2 | 0.9 |
| Active Ingredient | 0.2 | 0.2 | 0.5 | 0.2 | 0.9 |
| PEG 400 | 20 | 50.3 | 50 | 55 | 99.0 |
| PEG 4000 | 10 | 25 | 25 | 20 | — |
| Water | 2 | 2 | 2 | 2 | — |
| glycerol monostearate | 8 | 8* | 8* | 8* | — |
| White Petrolatum | 49.7 | 4.7 | 4.4 | 4.7 | — |
| Vitamin E | 0.1 | 0.1 | 0.1 | 0.1 | 0.1 |
| Isopropyl Myristate | 10 | 10 | 10 | 10 | — |
| Total | 100 | 100 | 100 | 100 | 100 |

*Glycerol monostearate, mono and diglycerides, NF sold under the tradename Geleol ™ is the glycerol monostearate used in formulations C1, C2 and C3.

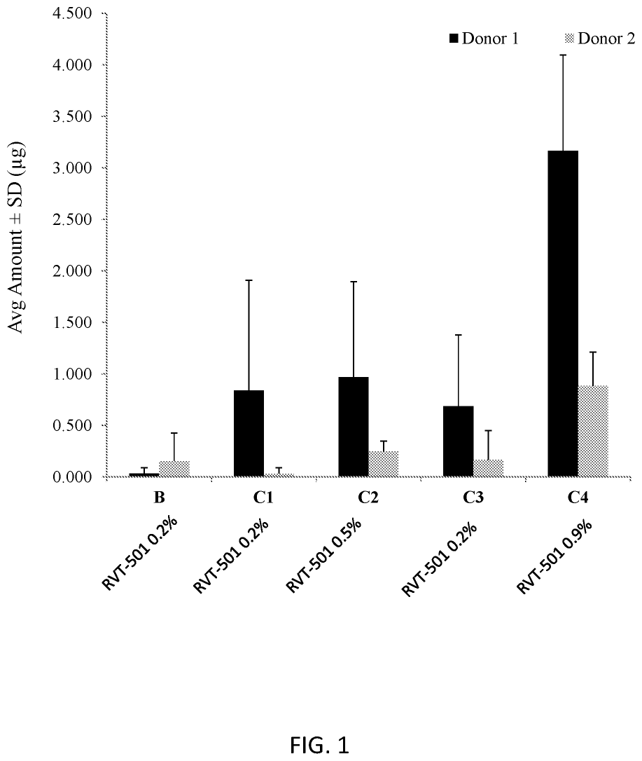

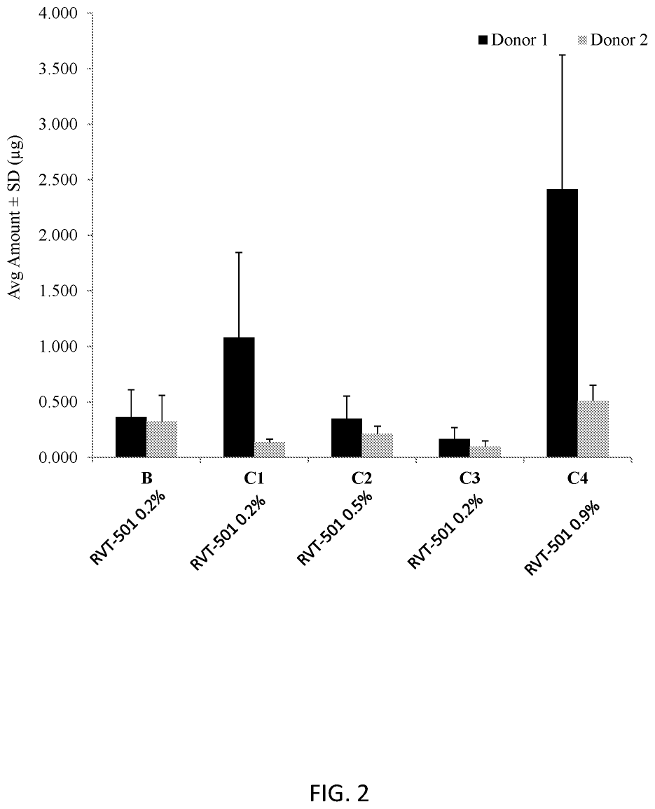

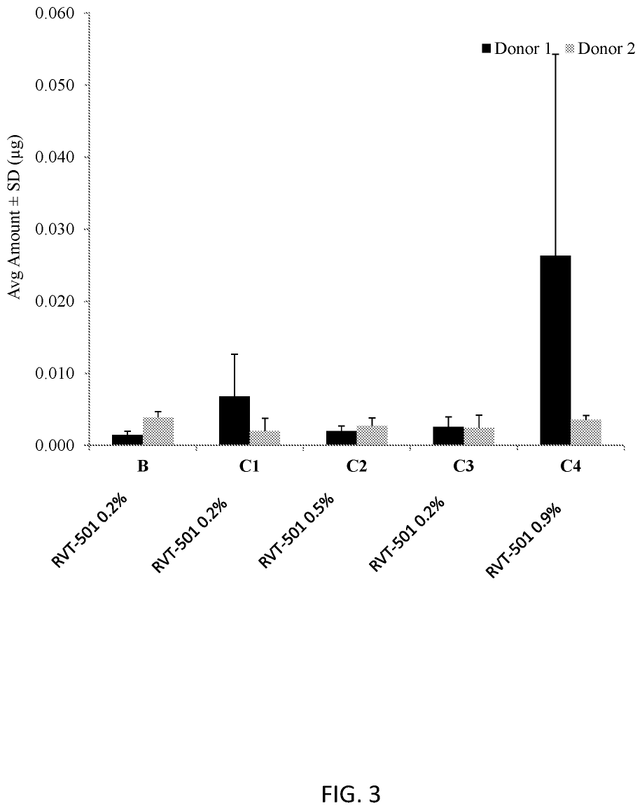

The objective of this study was to evaluate the penetration of methyl N-[3-(6,7-dimethoxy-2-methylaminoquinazolin-4-yl)phenyl]terephthalamic acid (RVT-501) into and across human cadaver skin from 4 formulations (B, C1, C2, and C3) and 1 drug solution (C4). The results indicated greatest permeation of methyl N-[3-(6,7-dimethoxy-2-methylaminoquinazolin-4-yl)phenyl]terephthalamic acid (RVT-501) from the PEG-400 solution (C4). This was expected since C4 was used as a positive control in the study. Drug levels were below the limit of quantification in receptor media at 24 hours for all formulations tested. Results from donor 1 suggested C1 to have higher permeation compared to B, C2, and C3. However, donor 2 results suggested the three formulations to have nearly equivalent permeation. Overall, donor 1 showed a trend of higher permeation compared to donor 2. It was noted that donor 1 appeared visually thinner than donor 2. In addition, a dose response was not observed between the two strengths, 0.2% and 0.5%. Since a similar trend of C1 having greater permeation was not observed in both donors, it can be concluded that formulations B, C1, C2, and C3 had nearly equivalent permeation into the stratum corneum (FIG. 1), epidermis (FIG. 2), and dermis (FIG. 3).

### Example 2: Treatment of Atopic Dermatitis

Atopic dermatitis (AD) was induced in specific pathogen-free (SPF) female NC/Nga mice (n=8/group), 8-12 weeks old, by repeated percutaneous applications of dinitrochlorobenzene (DNCB) to the dorsal skin of the ears and back on days 4, 7, 10, and 13. NC/Nga mice are an established mouse model for atopic dermatitis. See Suto et al. NC/Nga mice: a mouse model for atopic dermatitis; Int Arch Allergy Immunol. 1999; 120 Suppl 1:70-5; and Gao et al., Establishment of allergic dermatitis in NC/Nga mice as a model for severe atopic dermatitis, Biol. Pharm. Bull. 2004 September; 27(9): 1376-81.

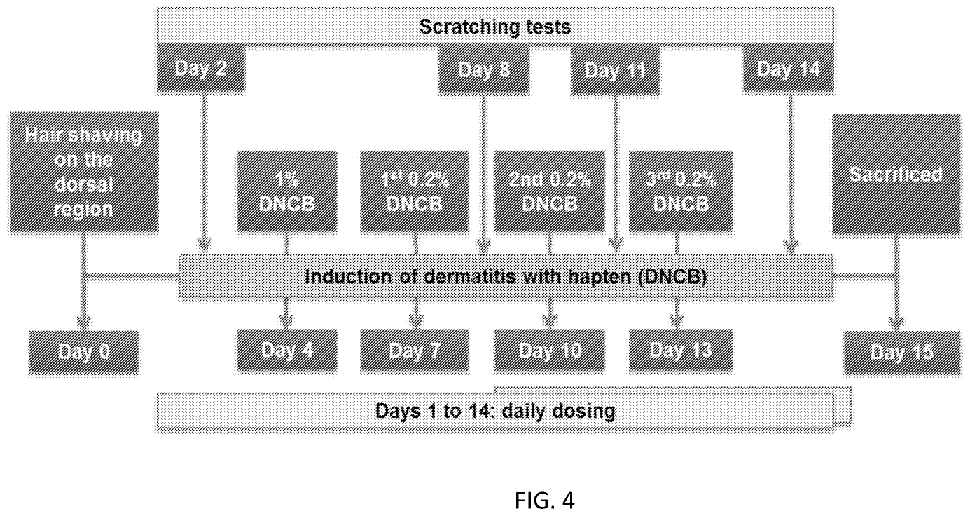

A prophylactic study and a therapeutic study was conducted:
1. Prophylactic study: 0.2% formulation (C1), 0.5% formulation (C2), RVT-501 placebo, tacrolimus placebo, 0.1% tacrolimus, or no treatment (AD control) on days 1-14 or sham-induction of AD.
2. Therapeutic study: 0.2% formulation (C1), 0.5% formulation (C2), active ingredient placebo, tacrolimus placebo, 0.1% tacrolimus, or no treatment (AD control) on days 8-14. See FIG. 4.

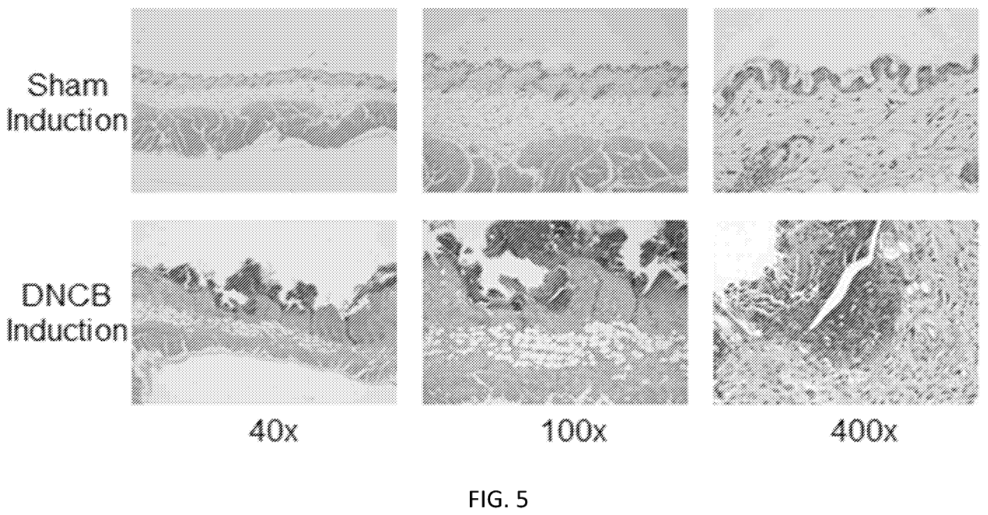

Scratching assays were performed on days 2, 8, 11, 14 in both studies. Skin samples were harvested for histopathology and cytokine analysis on day 15. Histopathology of sham-induced versus DNCB-induced mouse skin indicates clear presence of atopic dermatitis. See FIG. 5.

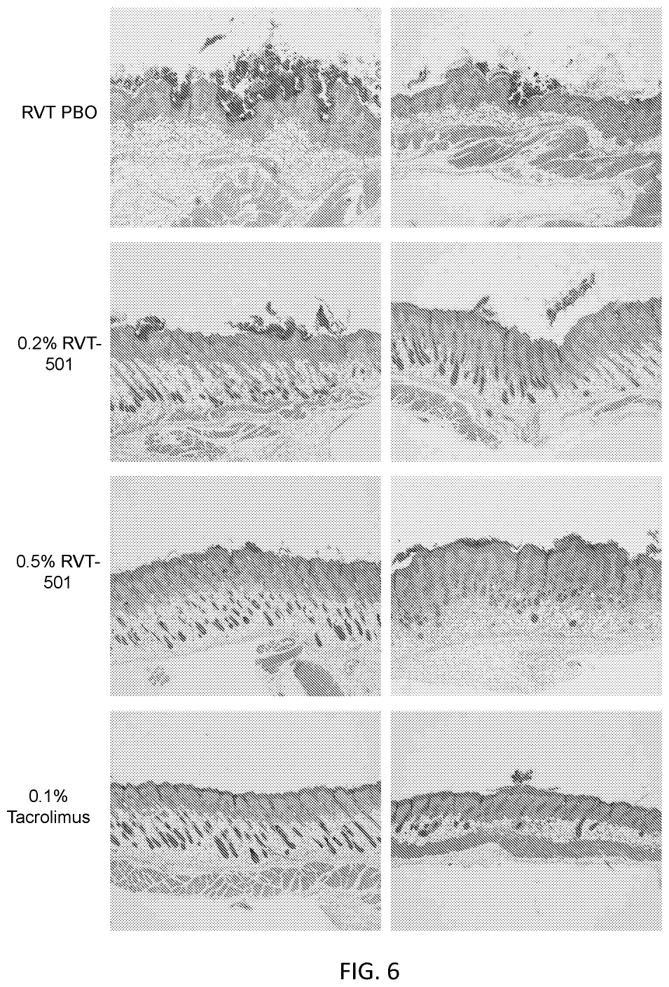

Skin sections were examined at day 15 for AD-associated pathology. Prophylactic treatment with 0.5% formulation (C2) or 0.1% tacrolimus attenuated AD lesions induced by DNCB at the microscopic level. See FIG. 6, left column. As a therapeutic treatment, 0.5% formulation (C2) and 0.1% tacrolimus trended toward a reduction in AD lesion severity. See FIG. 6, right column.

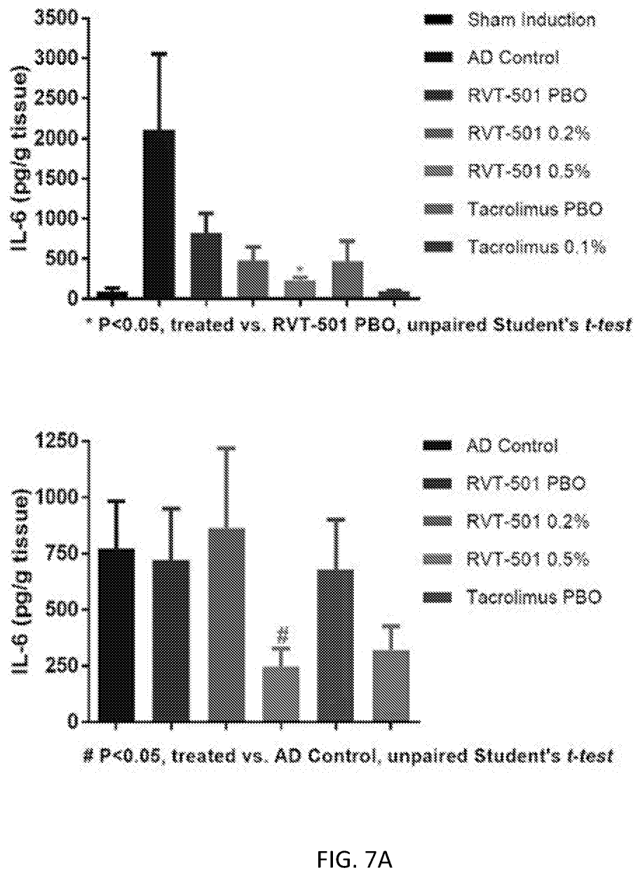

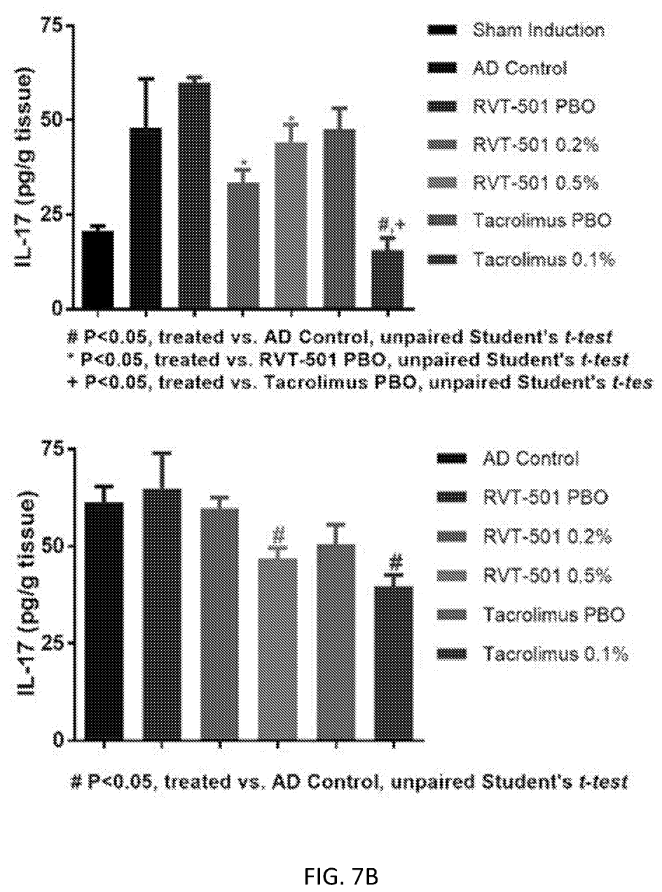

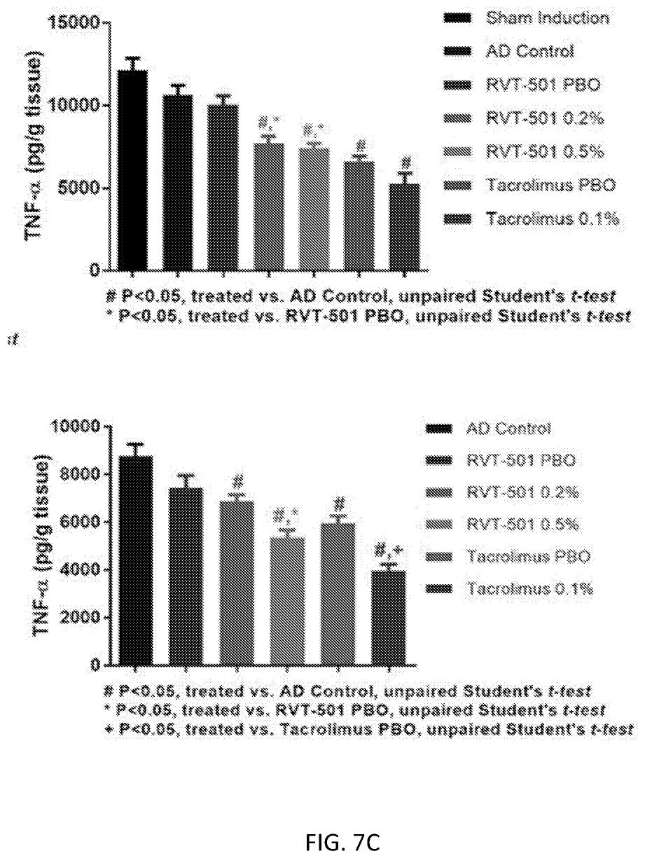

Skin sections were harvested for cytokine analysis at the end of each study to interrogate how these immune modulators were affected by the different treatments. Prophylactic administration of methyl N-[3-(6,7-dimethoxy-2-methylaminoquinazolin-4-yl)phenyl]terephthalamic acid (RVT-501) significantly reduced G-CSF, GM-CSF, KC, MIP-1α, and TNF-α in a dose-dependent manner. Additionally, the 0.5% formulation (C2) decreased IL-3, IL-6, IL-17, MCP-1, and MIP-1β. Therapeutically, Il-1β showed a significant dose-dependent decrease with methyl N-[3-(6,7-dimethoxy-2-methylaminoquinazolin-4-yl)phenyl]terephthalamic acid (RVT-501) treatment. Significant decreases with the 0.5% formulation (C2) were also seen with IL-3, eotaxin, G-CSF, GM-CSF, KC, MIP-1α, MIP-1β, and TNF-α. As a therapeutic, 0.1% tacrolimus significantly decreased IL-1α, IL-1β, IL-4, IL-5, IL-10, IL-12(p40), IL-13, eotaxin, GM-CSF, KC, MCP-1, MIP-1α, MIP-1β, RANTES, and TNF-α. Reduction of these inflammatory cytokines and chemokines likely contributes to the reduction in immune cell infiltration as seen via histopathology in both studies with 0.5% formulation (C2) and 0.1% tacrolimus. See FIG. 7.

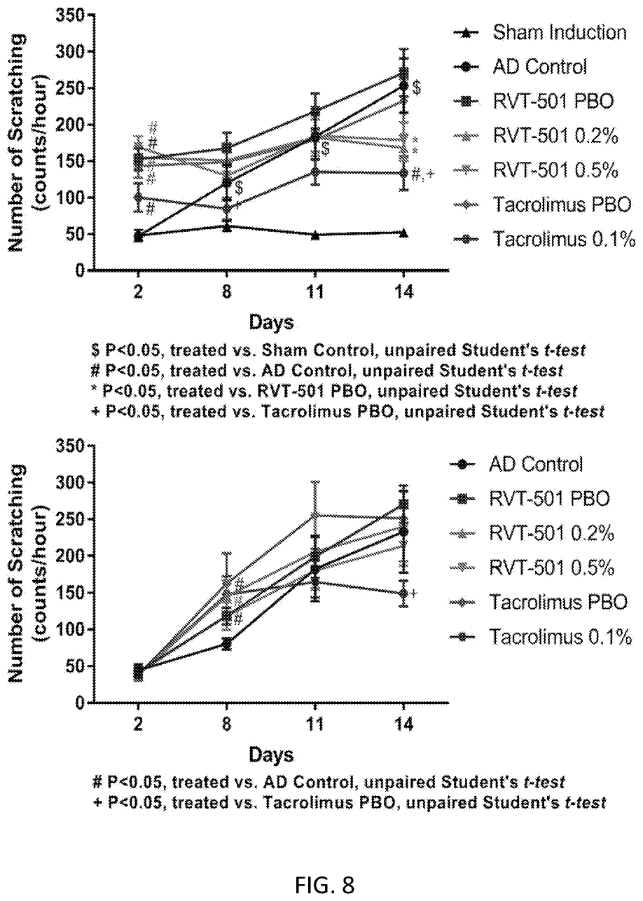

All treatments groups show a significant reduction in scratching relative to placebo in the prophylactic study. As a therapeutic, 0.1% tacrolimus showed a significant decrease in scratching at day 14. See FIG. 8.

CONCLUSIONS: The prophylactic study showed that RVT-501 0.5% formulation (C2) significantly reduced skin ulceration and preserved skin architecture when compared to active ingredient placebo controls and AD control animals. RVT-501 0.5% formulation (C2) also significantly reduced D14 scratching events, ear thickness, AD skin lesion score, and multiple AD-related pro-inflammatory cytokines when compared to the RVT-501 placebo; all of which appeared to reflect dose dependent responses from the 0.2% to 0.5% formulations (C1 and C2, respectively). The therapeutic study showed significant reduction in AD skin lesion score versus the active ingredient placebo that appeared dose dependent, as well as trends in decreased ulceration and ear thickness with RVT-501 0.5% formulation (C2), though these latter changes did not reach statistical significance. Therapeutic treatment of the established mouse AD lesions also revealed significant decreases in AD-related pro-inflammatory cytokines, though these effects were not as prominent as the 14 day prophylactic treatment.

In summary, significant reductions in scratching, microscopic skin histopathology, and inflammatory cytokines were observed with the RVT-501 0.5% formulation (C2) and 0.1% tacrolimus administered prophylactically. Trends toward significance were seen with RVT-501 0.5% formulation (C2) administered therapeutically, and may have been achieved in a model where longer treatment is possible. Accordingly, topical methyl N-[3-(6,7-dimethoxy-2-methylaminoquinazolin-4-yl)phenyl]terephthalamic acid (RVT-501) appears to be an effective treatment for atopic dermatitis.

Although the present invention has been described in considerable detail with reference to certain preferred embodiments thereof, other versions are possible. Therefore, the spirit and scope of the appended claims should not be limited to the description and the preferred versions contained within this specification.

### Example 3: Phase 2 Study of RVT-501 in Adult and Adolescent Subjects with Atopic Dermatitis

Atopic dermatitis is a chronic inflammatory disease of the skin characterized by intense itch (pruritus) and eczematous lesions. It is one of the most common skin diseases, affecting 10-20% of the population in developed countries. It occurs more commonly in children, affecting 15-30% of the child population, and recent estimates indicate approximately 10% of adults are affected. Of the pediatric population, approximately 60% of patients present in the first year of life, and about 85% of patients present by 5 years old.

Disease is mild to moderate in most patients, with 70% of all patients, and 80% of children having mild to moderate disease, and 20% of patients having moderate to severe disease, where the clinical features are more intense and relapsing. Many factors, both genetic and environmental, contribute to the pathogenesis of the disease, which is characterized by defects in skin barrier and immune system dysregulation. The skin lesions that result from these defects are itchy, painful, and cause the patient social and psychological harm due to their appearance. Beyond the immediate physical symptoms and psychological manifestations of the AD lesions, the disease has profound secondary effects on the well-being of patients. Specifically, pruritus associated with the causes the significant patient discomfort, often leading to sleep deprivation, which manifests also into poor sleep quality in parents of young patients.

Despite the high prevalence, there are limited current treatment options available for the patients. The first line treatment option for patients with mild-moderate disease is topical corticosteroids, but many patients are steroid refractory and there are significant long-term safety risks associated with their use. Topical calcineurin inhibitors, Elidel and Protopic, are used as a second-line treatment option but have a boxed warning for carcinogenicity risks. Thus, there is a significant unmet medical need for a therapeutic that is both safe and efficacious.

The diagnosis criteria for atopic dermatitis requires at least three of the following major criteria: pruritus, typical morphology and distribution (Adults: flexural lichenification or linearity, Children and infants: involvement of facial and extensor surfaces), chronic or chronically relapsing dermatitis, or personal or family history of atopy (asthma, allergic rhinitis, atopic dermatitis). As well as at least three of the following minor criteria: xerosis, ichthyosis/keratosis pilaris/palmar hyperlinearity, immediate (type 1) skin test reactivity, elevated serum IgE, early age at onset, tendency to skin infections (Staphylococcus aureus, herpes simplex)/impaired cellular immunity, tendency to nonspecific hand/foot dermatitis, nipple eczema, cheilitis, recurrent conjunctivitis, Dennie-Morgan infraorbital fold, keratoconus, anterior subcapsular cataracts, orbital darkening, facial pallor/erythema, Pityriasis alba, anterior neck folds, itch when sweating, intolerance to wool and lipid solvents, perifollicular accentuation, food intolerance, course influenced by environmental/emotional factors, or white demographic/delayed blanch.

RVT-501, previously known as E6005, is an investigational phosphodiesterase 4 (PDE4) inhibitor. Studies have shown that PDE4 activity is upregulated in atopic dermatitis, resulting in reduced levels of cAMP, ultimately causing protein kinase A (PKA) dependent elevation in pro-inflammatory cytokines. Preclinical and clinical data support that PDE4 inhibition by RVT-501 results in downregulation of disease-related cytokines as well as resultant attenuation in disease severity.

Eisai Co., Ltd. developed a version of RVT-501 ointment (Formulation B), which was a white petrolatum based composition. Four concentrations (0.01%, 0.03%, 0.1%, 0.2%) of RVT-501 Formulation B were developed. The RVT-501 ointment (Formulation B) was used in nonclinical and clinical studies completed to date. The degree of efficacy observed in prior clinical studies with the highest concentration RVT-501 0.2% ointment did not appear to reach a maximum; thus, a formulation with an increased concentration of RVT-501 was developed by Eisai (Formulation C) utilizing a polyethylene glycol based composition. Further refinement of this Formulation C by Dermavant Sciences has resulted in a new RVT-501 ointment formulation at two concentrations, RVT-501 0.2% ointment (Formulation C) and RVT-501 0.5% ointment (Formulation C). These two concentrations of formulation C will be used in this study.

Three studies conducted in adult and pediatric subjects with AD included efficacy endpoints as secondary objectives. In general, these studies showed that RVT-501, at various doses, has dose-related increasing efficacy based on various scoring systems in atopic dermatitis with little to no systemic exposure to RVT-501 or M11 observed.

Study 001 was a multiple ascending dose study in healthy Japanese male subjects that consisted of an open-label, vehicle-controlled skin irritation period (patch test and photopatch test) and a multiple ascending dose period. The patch/photopatch component of the study was performed using Finn Chambers® containing nothing, white petrolatum, vehicle, 0.01%, 0.03%, 0.1% or 0.2% RVT-501 ointment. In the multiple ascending dose component, subjects randomly received vehicle ointment or 0.01%, 0.03%, 0.1% or 0.2% RVT-501 ointment once daily (QD) or twice daily (BID) for up to 11 days (approximately 5 g over approximately 10% BSA). No significant skin or systemic AEs were identified in this study.

A Phase 1/2 study of RVT-501, Study 101, in Japanese male subjects aged 20 to 64 years with AD, was conducted primarily to evaluate the safety and PK of topical application of RVT-501 ointment (0.01%, 0.03%, 0.1%, and 0.2%) compared with vehicle after application for up to 10 days. Additional exploratory objectives included the efficacy of topical application with these concentrations of RVT-501 ointment in the same population. The severity scores of targeted eczema (SSTE) on the back were significantly reduced at the end of the study compared to baseline, in the 0.03%, 0.1%, and 0.2% RVT-501 ointment groups (P=0.031 in the 0.1% RVT-501 group, P<0.001 in the 0.03% and 0.2% RVT-501 groups). The least squares mean difference from vehicle at the end of the study was statistically significant in the 0.2% RVT-501 ointment group (P=0.003). Similar findings, including dose-dependent efficacy responses, were noted for other efficacy measures such as eczema area and severity index (EASI) and scoring atopic dermatitis (SCORAD) in this study.

In Study 201, 78 adults aged 20 to 64 with mild to moderate AD encompassing 5 to 30% body surface area (BSA) were randomized 2:1 to RVT-501 0.2% (n=52) or control (n=26) ointment BID for 4 weeks. All subjects were then continued on RVT-501 0.2% ointment BID for an additional 8 weeks. A total of 72 subjects were exposed to 0.2% RVT-501 ointment in Study 201. Subjects initially receiving RVT-501 0.2% ointment had greater improvements in the Eczema Area and Severity Index (EASI) and Scoring Atopic Dermatitis (SCORAD) scores versus those receiving vehicle, but none of the comparisons of RVT-501 vs. vehicle reached statistical significance at Week 4. Additionally, all subjects generally saw continued trends towards improvement in AD during the 8-week extension phase.

Study 102 was a Phase 1/2 multicenter, randomized, vehicle-controlled study wherein 62 pediatric subjects aged 2 to 15 years with mild to moderate AD were enrolled in sequential, decreasing-aged cohorts and treated with control ointment, or 0.05% or 0.2% RVT-501 ointment BID for 14 days. Improvements in SSTE and Investigator's Global Assessment were consistently seen for subjects on RVT-501 0.2% ointment vs. vehicle, but similar improvements were not see with RVT-501 0.05% ointment. Dose-dependent improvements in AD severity were observed, as were improvements in pruritus in a subject cohort that was not on concomitant antihistamine or anti-allergic medication.

To date, there have been no reports of serious adverse events related to RVT-501.

According to the results of Study 001 in healthy adult males and Study 101 in male adults with atopic dermatitis, RVT-501 ointment produced no clinically-significant findings at concentrations of 0.01% to 0.2% RVT-501 in terms of skin irritation (patch test, photopatch test), other adverse events, laboratory values, vital signs, 12-lead electrocardiography, or ophthalmological findings.

In Study 201, conducted in adult subjects with atopic dermatitis, rates of noteworthy adverse events (e.g., adverse events at the administration site and adverse events involving skin infections) were similar in the vehicle ointment and 0.2% RVT-501 ointment groups during the 4-week randomization stage. In addition, although some adverse events occurred more often in the 0.2% RVT-501 ointment group than in the vehicle ointment group, all were mild or moderate, and tolerability was good. The safety profile for 0.2% RVT-501 ointment applied for 12 weeks was largely the same as that applied for 4 weeks.

In the 102 Study, conducted in pediatric subjects with atopic dermatitis, repeated application of 0.05% and 0.2% RVT-501 ointment for 2 weeks produced no adverse events considered attributable to the investigational product. Furthermore, there were no other clinically significant findings in terms of other laboratory values, vital signs or 12-lead electrocardiography.

The safety of repeated administration of RVT-501 ointment has not yet been evaluated beyond 12 weeks in adults or beyond 2 weeks in children.

The study objectives are to evaluate the safety, pharmacokinetics and efficacy of multiple doses of RVT-501 topical ointment. Prior clinical studies have shown significant efficacy in pediatric patients (Study 102) and positive although nonsignificant efficacy results in adult patients (Study 201) with a 0.2% topical ointment. Preclinical and clinical dose-ranging evidence suggests that higher concentration formulations may result in enhanced efficacy. The primary objective of this study is to evaluate the safety and pharmacokinetics of a 0.5% formulation—a higher concentration formulation than has been used previously—in both adults and adolescents in a BID dosing regimen. A 0.2% BID formulation arm will be included to control for efficacy and safety findings at the previous dose level.

Previous clinical studies with topical ointment doses up to 0.2% BID have shown dose dependent improvement in signs and symptom associated with AD in pediatric and adult populations. In addition, RVT-501 has been well tolerated with few skin related or systemic AEs and has minimal systemic absorption. Preclinical and clinical dose-ranging studies support dosing beyond 0.2%, the highest concentration ointment previously tested. Recently performed skin penetration studies showed an increase in RVT-501 in the skin following topical application of 0.5% ointment compared with the previous 0.2% formulation. Further, radiolabelled pharmacokinetics studies where 14C-RVT-501 was dermally delivered to stripped skin of non-fasted male rats showed that, after a single application, radiolabelled RVT-501 was present in the skin application site 24 hours after administration at 75% of the levels measured after 30 minutes (maximum radioactivity). Thus, both 0.2% and 0.5% topical formulations, BID, will be tested for comparative efficacy in both adults and adolescents.

RVT-501-2001/Phase 2 Study of RVT-501 in Adult and Adolescent Subjects with Atopic Dermatitis:

Primary Objective: To evaluate the safety and pharmacokinetics of topical RVT-501 in adult and adolescent subjects with atopic dermatitis. Primary endpoints: Plasma concentrations of RVT-501 and M11 metabolite, pharmacokinetic parameters (if data permit). Frequency and severity of adverse events (local and systemic), laboratory values, vital signs, and ECG. Secondary Objective: To assess the efficacy of topical RVT-501 in adult and adolescent subjects with atopic dermatitis. Secondary endpoints: Efficacy as determined by: Change from baseline in Investigators Global Assessment (IGA), Proportion of subjects who achieve an IGA of 0 or 1 and at least a decrease of 2 point in IGA, Change from baseline in BSA, Change from baseline in Eczema Area and Severity Index (EASI) score, EASI-50 Analysis (50% reduction in EASI score from baseline), Change from baseline in pruritus as measured with the Numeric Rating Scale. Number of Subjects planned: Approximately 150 total of which approximately 90 will be adults (ages 18 to 70) and 60 will be adolescents (ages 12 to 17). Study design: Multi-center, randomized, vehicle-controlled, double-blind trial. Subjects will be randomized (1:1:1) to the following: RVT-501 0.2% ointment BID×28 days (30 adults, 20 adolescents), RVT-501 0.5% ointment BID×28 days (30 adults, 20 adolescents), Vehicle ointment BID×28 days (30 adults, 20 adolescents). Adult subjects will be enrolled first. After an interim review of the data in 60 adult subjects, adolescent subjects ages (12 to <18) may be enrolled. Duration of the treatment will be for 28 days.

This was a multicenter, randomized, vehicle-controlled, double-blind Phase 2 study in adults and adolescent subjects with mild to moderate AD.

All subjects underwent screening procedures within 30 days of enrollment to confirm eligibility. At Day 0 (baseline), eligible subjects were randomized (1:1:1) to one of three treatment arms. Subjects were instructed on how to apply RVT-501 or placebo while under the supervision of site personnel in the clinic. Subjects applied a thin layer of study medication with their fingertip to all affected areas. Study medication was dispensed to subjects and was applied at home as instructed by site personnel between clinic visits.

During the treatment period, subjects applied RVT-501 ointment or vehicle to affected areas twice daily (BID) for 28 days. Subjects returned to the clinic at Day 4 for evaluation, and again at Weeks 1, 2, 3, and 4 for safety and efficacy assessments. Pharmacokinetic samples were collected at Weeks 1 and 4. On clinic visit days (except on the Day 4 visit), subjects applied study drug on-site while under the supervision of site personnel, after efficacy assessments had been completed.

There was a Follow-up visit 7-10 days following the end of study treatment. A subject's total participation in the study included 8 clinic visits over the course of approximately 10 weeks.

Target Population: Approximately 150 subjects (90 adults and 60 adolescents) with mild or moderate AD were planned to be enrolled.

Main criteria for inclusion: Males and females with confirmed diagnosis of AD by Hanifin and Rajka criteria. For adult subjects, the age range was 18 to 70 years. For adolescent subjects, the age range was 12 to 17 years. Subjects with AD covering ≥3% and <40% of the body surface area (BSA) and with an Investigator's Global Assessment (IGA) of 2 or 3 (mild or moderate) at baseline. Scalp, palms, and soles were excluded from the BSA calculation to determine eligibility at baseline. Minimum Eczema Area and Severity Index (EASI) score of 7 at baseline. AD present for at least 12 months according to the patient/care giver and stable disease for at least 1 month according to the patient/care giver.

Compound: RVT-501 0.2% ointments, applied BID for 28 days, Formulation C1 (see Table 1). RVT-501 0.5% ointments, applied BID for 28 days, Formulation C2 (see Table 1). Vehicle ointment, applied BID for 28 days, Formulation B (see Table 1).

Criteria for Evaluation: Primary Outcome Measures: Frequency and severity of adverse events (local and systemic), laboratory values, vital signs and ECGs, Plasma concentrations of RVT-501 and M11 metabolite, and pharmacokinetic parameters (if data permit). Secondary Outcome Measures: Efficacy as determined by the change form baseline in Investigators Global Assessment (IGA), change from baseline in EASI score, proportion of subjects who achieve an IGA of 0 or 1, and at least a 2-point decrease in IGA, proportion of subjects who achieve an IGA of 0 or 1, change from baseline in BSA affected, EASI-50 Analysis (attaining at least a 50% reduction in EASI score from baseline), and change from baseline in Pruritus as measured with the numeric rating score (NRS) using a visual analog scale. Exploratory: Change from baseline in Patient Oriented Eczema Measure (POEM), patient reported outcome measure(s).

Statistical Methods: Efficacy Analyses: A sample size and power sensitivity analysis was conducted for the efficacy endpoints. Assuming an effect size (defined as difference of mean change from baseline EASI score between treatment groups relative to pooled standard deviation) of 0.7, a sample size of 50 subjects in an active arm and 50 subjects in the combined placebo will provide 93% power at an alpha level of 0.05 (2-sided), based on a 2-sided t-test. The sample size will also allow a difference of 33% between placebo and active treatment in a responder endpoint to be detected with 90% power and a 0.05 significance level assuming the proportion of responders in the placebo group is <=20%. Efficacy endpoints will be summarized and listed by treatment for each age group and both age groups combined; The between-treatment comparisons (active vs placebo and between active dose groups) for continuous efficacy variables will be performed using an analysis of covariance (ANCOVA) model. The between treatment comparisons for the proportion of responders will be compared using a CMH or Chi-square test. Safety Analyses: Adverse events will be mapped to a Medical Dictionary for Regulatory Activities (MedDRA). Treatment emergent adverse events will be summarized by treatment, preferred term and system organ classification. Descriptive summaries of vital signs, ECG parameters, and clinical laboratory results will be presented by study visit and treatment group. Pharmacokinetic Analyses: RVT-501 and M11 plasma concentrations will be listed by subject, treatment, and time; and will be summarized by treatment and time. The number and percent of subjects with a measurable concentration of either analyte at each time point and any time during the study will be provided.

The last observation carried forward (LOCF) was implemented in the case of missing data.

The total and regional EASI scores were summarized by visit, and for the change from baseline and percent change from baseline. The proportion of subjects achieving at least 50% reduction in EASI score from baseline was also summarized. The between-treatment comparisons of change and percent change from baseline were performed by visit using an analysis of covariance (ANCOVA) model. The baseline EASI score was included as a covariate. The age group was included as a covariate for the analyses based on the combined groups. The differences with 95% CIs and p-values between each active and the vehicle group were presented.

The IGA scores were summarized for the shift from baseline by treatment group and visit. The proportion of subjects with an IGA score of 0 (clear) or 1 (almost clear) and at least a 2-point reduction from the baseline at Week 4, and the proportion of subjects with an IGA score of 0 or 1 at Week 4 were summarized.

For the IGA scores, the between-treatment comparisons were performed using an ANCOVA model similar to the model used for the EASI score.

The IGA responder endpoint was defined as IGA score of 0 or 1, and with at least a 2-point reduction from the baseline value at Week 4.

Pairwise comparisons of treatment groups (RVT-501 0.2% vs. vehicle and RVT-501 0.5% vs. vehicle) were generated using the Dunnett's procedure of adjustment for multiple comparisons, and statistical significance of the treatment effect was assessed at the two-sided 5% level.

The total affected BSA and NRS for pruritus were summarized by visit, and for the change from baseline and percent change from baseline.

Interim Analyses: When approximately 60 adult subjects had completed Week 4 of the study, safety and efficacy data were to be reviewed prior to randomization of adolescent subjects. This review did not include subject level data and covered the AEs, clinical laboratory results, ECGs, and vital signs. PK data was also reviewed as well as IGA/EASI results. The review was conducted by clinical research personnel not directly involved with the conduct of the study.

Analysis Populations: Four analysis populations were used for this study. The Safety population, consisting of all subjects enrolled into the study who received study drug, was used for the safety analyses. The intent-to-treat (ITT) population, defined as all subjects randomized to treatment, was the primary population used for the efficacy analyses. The per protocol (PP) population included subjects who applied at least 50% of the doses. The PP population was used for confirmatory analysis of the efficacy variables. The PK population included all subjects who underwent plasma PK sampling and had at least one evaluable PK sample (a concentration reported as below the lower limit of quantitation (LLQ) of the assay was considered an evaluable PK sample).

Safety Analyses: Treatment-emergent adverse events (TEAEs) were listed by subject and summarized by the number of subjects reporting the events, as well as by system organ classification, preferred term, severity, seriousness, and relationship to the study drug. All TEAEs tables were presented separately for adults, adolescents, and overall.

The clinical laboratory results were listed individually by visit, and abnormal values were summarized with the clinical significance. Raw values and change from baseline were summarized by visit. Vital signs were listed and presented descriptively (including change and percent change from baseline) by visit. Results of the single 12-lead ECGs were listed and summarized by visit.

Overall Design: This is a multi-center, randomized, vehicle-controlled, double-blind Phase 2 study in adults and adolescent subjects with mild to moderate atopic dermatitis. All subjects will undergo screening procedures within 30 days of enrollment to confirm eligibility. At Day 0 (baseline), eligible subjects will be randomized (1:1:1) to one of three treatment arms. Subjects will be instructed on how to apply RVT-501 while under the supervision of site personnel in the clinic. Briefly, subjects should apply a thin layer of study medication with their fingertip to all affected areas. Study medication will be dispensed to subjects and will be applied at home as instructed by site personnel between clinic visits. During the treatment period, subjects will apply RVT-501 ointment to affected areas twice a day for 28 days. Subjects will return to the clinic at Day 4 for evaluation, and again at Weeks 1, 2, 3 and 4 for PK, safety and efficacy assessments at the timepoints noted in the Time and Events Table. On clinic visit days (except on Day 4 visit), subjects should apply study drug on-site while under the supervision of site personnel, after efficacy assessments have been completed.

There will be a follow-up visit 7-10 days following the end of study treatment. A subject's total participation in the study will include 8 clinic visits over the course of approximately 10 weeks.

Treatment arms and duration—Treatment Group A: RVT-501 0.2% ointment twice daily×28 days, Treatment Group B: RVT-501 0.5% ointment twice daily×28 days, Treatment Group C: Vehicle ointment twice daily×28 days.

Table 2 provides the timeline for events throughout the treatment period.

TABLE 2
Time and Events over treatment period

| | | | | | | | | Follow-up |
|---|---|---|---|---|---|---|---|---|
| | Screening | Treatment Period | | | | | | 7-10 days |
| | (up to 30 | | Day 4 | Week 1 | Week 2 | Week 3 | Week 4 | post-dose |
| | days prior | | (+/−1 | (Day 7 +/− | (Day 14 +/− | (Day 21 +/− | (Day 29 +/− | or Early |
| Procedure | to Day 0) | Day 0 | day) | 2 days) | 2 days) | 2 days) | 1 days) | Termination |
| Informed consent | X | | | | | | | |
| Inclusion and | X | | | | | | | |
| exclusion criteria | | | | | | | | |
| Demography | X | | | | | | | |
| Brief physical exam | X | | | | | | | X |
| (include height and | | | | | | | | |
| weight at screening | | | | | | | | |
| only) | | | | | | | | |
| Medical history | X | | | | | | | |
| (includes substance | | | | | | | | |
| usage [and Family | | | | | | | | |
| history of premature | | | | | | | | |
| CV disease]) | | | | | | | | |
| Substances = drugs, | | | | | | | | |
| alcohol, tobacco and | | | | | | | | |
| caffeine | | | | | | | | |
| Fitzpatrick skin type | | X | | | | | | |
| assessment | | | | | | | | |
| Pregnancy test | X | X | | X | X | X | X | X |
| (WCBP) | | | | | | | | |
| Serum at Screen, | | | | | | | | |
| Urine at other visits | | | | | | | | |
| HIV, Hep B and Hep | X | | | | | | | |
| C Screen | | | | | | | | |
| Laboratory | X | X | | X | | | X | X |
| assessments | | | | | | | | |
| (chemistry and | | | | | | | | |
| hematology, | | | | | | | | |
| urinalysis including | | | | | | | | |
| liver chemistries) | | | | | | | | |
| 12-lead ECG (pre- | X | X | | X | | | X | |
| dose on dosing | | | | | | | | |
| days) | | | | | | | | |
| Vital signs (pre-dose | X | X | | X | | | X | X |
| on dosing days) | | | | | | | | |
| Randomization | | X | | | | | | |
| On-site training on | | X | | | | | | |
| drug application | | | | | | | | |
| RVT-501 | | X | | X | X | X | X | |
| administration in- | | | | | | | | |
| clinic under site | | | | | | | | |
| supervision | | | | | | | | |
| Study Treatment | | X | | X | X | X | X | |
| Dispensation/Collection | | | | | | | | |
| Dispense, collect, | | X | X | X | X | X | X | |
| review subject | | | | | | | | |
| diaries | | | | | | | | |
| PK samples ¹ | | | | X | | | X | |
| AE/SAE review | | X | X | X | X | X | X | X |
| Concomitant | X | X | X | X | X | X | X | X |
| medication review | | | | | | | | |
| Investigator's Global | X | X | X | X | X | X | X | X |
| Assessment (IGA) | | | | | | | | |
| (pre-dose) | | | | | | | | |
| EASI Score (pre- | X | X | X | X | X | X | X | X |
| dose) | | | | | | | | |
| BSA excluding | X | X | | | | | | |
| scalp, palms, and | | | | | | | | |
| soles | | | | | | | | |
| Whole body BSA | | X | X | X | X | X | X | X |
| Pruritis NRS (pre- | | X | X | X | X | X | X | X |
| dose) | | | | | | | | |
| Patient Reported | | X | X | X | X | X | X | X |
| Symptoms of | | | | | | | | |
| burning and itching | | | | | | | | |
| (pre-dose) | | | | | | | | |
| Patient Reported | | X | | | | | X | X |
| Outcomes: | | | | | | | | |
| POEM (pre-dose) | | | | | | | | |
| Clinical Photography | | X | | | | | X | X |
| (if applicable) | | | | | | | | |

¹ PK samples will be collected pre-dose at week 1 for all subjects. At week 4, PK samples will be collected pre-dose and within 2-4 hours post-dose.

Atopic Dermatitis Assessments: Efficacy measurement outcomes will include: Investigator Global Assessment (IGA): The Investigator's Global Assessment (IGA) of Disease Severity will be assessed at every on-site study visit. The IGA is a global assessment of the current state of the disease. It is a 5-point morphological assessment of overall disease severity and will be determined according to the categories described below. In order to be eligible, subjects must have an IGA score of 2 or 3 at Baseline visit (Day 0). Table 3 describes the IGA scores.

TABLE 3
IGA Scoring Assessment

| Score | Category | Definition |
|---|---|---|
| 0 | Clear | Minor, residual discoloration, no erythema or |
| | | induration/papulation, no oozing/crusting |
| 1 | Almost | Trace, faint pink erythema with almost no |
| | clear | induration/papulation, no oozing/crusting |
| 2 | Mild | Faint pink erythema with induration/papulation |
| | disease | and no oozing/crusting |
| 3 | Moderate | Pink-red erythema with moderate induration/papulation |
| | disease | and there may be some oozing/crusting |
| 4 | Severe | Deep/bright red erythema with severe |
| | disease | induration/papulation with oozing/crusting |

Eczema Area and Severity Index (EASI): The Eczema Area and Severity Index (EASI) will be assessed at every study visit. It quantifies the severity of a subject's atopic dermatitis based on both lesion severity and the percent of BSA affected. The EASI is a composite score ranging from 0-72 that takes into account the degree of erythema, induration/papulation, excoriation, and lichenification (each scored from 0 to 3 separately) for each of four body regions, with adjustment for the percent of BSA involved for each body region and for the proportion of the body region relative to the whole body. A detailed procedure of EASI score calculation is: Four anatomic sites (head, upper extremities, trunk, and lower extremities) are assessed for erythema, induration (papules), excoriation and lichenification as seen on the day of the examination. The severity of each sign is assessed using a 4-point scale: 0=No symptoms, 1=Slight or Mild, 2=Moderate, 3=Marked or Severe. The area affected by atopic dermatitis within a given anatomic site is estimated as a percentage of the total area of that anatomic site and assigned a numerical value according to the degree of atopic dermatitis involvement as follows: 0=no involvement, 1=<10%, 2=10 to <30%, 3=30 to <50%, 4=50 to <70%, 5=70 to <90%, 6=90 to 100%. The EASI score is obtained by using the formula:

EASI=0.1(Eₕ+Iₕ+Eₓₕ+Lₕ)Aₕ+0.2(Eᵤ+Iᵤ+Eₓᵤ+Lᵤ)Aᵤ+0.3(Eₜ+Iₜ+Eₓₜ+Lₜ)Aₜ+0.4(Eₗ+Iₗ+Eₓₗ+Lₗ)Aₗ
Where E, I, Ex, L and A denote erythema, induration, excoriation, lichenification and area, respectively, and h, u, t, and 1 denote head, upper extremities, trunk, and lower extremities, respectively.

EASI-50 represents the subjects achieving a 50% reduction in EASI score from baseline.

Body Surface Area (BSA): The BSA affected by Atopic Dermatitis will be evaluated (from 0 to 100%) at every visit. The subjects scalp, palms and soles should be excluded from the calculations at screening and baseline to determine subject's eligibility. At Day 0 and subsequent visits, BSA of the whole body affected with Atopic Dermatitis will be used to assess efficacy of the study treatment. One subject's palm (excluding fingers) represents approximately 1%, head 10%, upper extremities 20%, trunk 30%, and lower extremities 40% of his/her total BSA.

NRS (Numerical Rating Scale) for Pruritus is a validated scale used to quickly assess pruritus severity, where 0 is no itch and 10 is the worst imaginable itch.

Clinical photography may be performed in a subgroup of subjects at selected study centers that possess the capabilities. This is not required of subjects for participation in the study. Informed consent/assent and photographic release will be required. The photographs may not be referred to by the investigator at any subsequent study visit for the purposes of grading. Photographs will be taken of a representative area of the subject's disease area. Photographs will be taken at the time points specified in the Time and Events Table. Three photographs of the selected skin area will be taken in a standardized fashion (i.e., same camera, angle, background, distance).

Patient Reported Symptoms: The subject will assess burning and pruritus at the application site during clinic visits using the following scale: Burning: 0 None (no burning sensation), 1 Mild (Mild burning sensation (not really bothersome)), 2 Moderate (Moderate burning sensation that is somewhat bothersome), 3 Severe (Intense burning sensation that cause a definite discomfort) and Pruritus: 0 None (no itching), 1 Mild (Mild itching sensation (not really bothersome)), 2 Moderate (Moderate itching sensation that is somewhat bothersome), 3 Severe (Intense itching sensation that cause a definite discomfort). This should be completed by the subject prior to other assessments or evaluations by site personnel, where possible.

Patient Report Outcomes: The Patient Oriented Eczema Measures (POEM—adult version) is a tool used for monitoring atopic dermatitis severity. It focuses on the illness as experienced by the patients. Measurements will be assessed at the time points indicated in Table 1. The full version of POEM is available for free download from the University of Nottingham at http://www.nottingham.ac.uk/research/groups/cebd/resources/poem.aspx.

Patient Diary: The self-administered sign and symptom severity diary (which is based on the content of the POEM) assesses the severity of disease-related signs and symptoms. Response options are on an 11-point NRS and range from 0 (Absent) to 10 (Worst Imaginable). Subjects will be asked to complete the diary each day using a recall period of the past 24 hours where possible. Question 1 of the diary will be used to assess itch. An electronic diary may be utilized.

Pharmacokinetics: Blood samples for PK analysis of RVT-501 and the M11 metabolite will be collected at the time points indicated in Table 1. The actual date and time of each blood sample collection will be recorded as well as the date and time of the last dose of study mediation. The timing of PK samples may be altered and/or PK samples may be obtained at additional time points to ensure thorough PK monitoring.

Table 4 provides the final subject disposition of the current study. Table 5 provides the demographics of the subjects in the current study.

Summary of Results

Study Disposition: After 58 adult subjects completed Week 4 of the study, safety and efficacy data were reviewed. This interim analysis was completed in February 2017, prior to randomization of adolescent subjects. Since the safety and efficacy profile of RVT 501 met the pre-defined criteria in the Interim Analysis Charter, the enrolment of adolescent subjects was allowed.

A total of 157 subjects were randomized in the study (95 adults and 62 adolescents); all were included in the ITT population and in the Safety population (53 subjects in the vehicle group [31 adults and 22 adolescents], 55 subjects [34 adults and 21 adolescents] in the RVT-501 0.2% group, and 49 subjects [30 adults and 19 adolescents] in the RVT-501 0.5% group). Six subjects withdrew consent, 3 subjects were lost to follow up, 2 subjects did not complete the study due to TEAEs, and 1 subject was discontinued because of travelling due to family emergency (other). The PP population included 142 subjects (49 subjects in the vehicle group [29 adults and 20 adolescents], 50 subjects [31 adults and 19 adolescents] in the RVT-501 0.2% group, and 43 subjects [28 adults and 15 adolescents] in the RVT-501 0.5% group). Thirteen subjects were excluded from the PP population for significant treatment noncompliance, and 2 subjects were excluded due to major protocol deviations. The PK population included 152 subjects (51 subjects in the vehicle group [30 adults and 21 adolescents], 53 subjects [32 adults and 21 adolescents] in the RVT-501 0.2% group, and 48 subjects [30 adults and 18 adolescents] in the RVT-501 0.5% group). Five subjects were excluded from the PK population due to missing PK samples.

A total of 145 subjects (87 adults and 58 adolescents) completed the study as planned (50 [94.3%] subjects in the vehicle group, 52 [94.5%] subjects in the RVT-501 0.2% group, and 43 [87.8%] subjects RVT-501 0.5% group).

Demographic and Baseline Characteristics: The mean baseline treatable BSA affected by AD was similar over the treatment groups (15.2% in the vehicle group, 15.9% in the RVT-501 0.2% group, and 13.5% in the RVT-501 0.5% group). Most of the subjects had an IGA of disease severity of 3 (moderate) at baseline.

TABLE 4
Subject Disposition

| | | |
|---|---|---|
| Vehicle | RVT-501 0.2% | RVT-501 0.5% |
| (N = 53) | (N = 55) | (N = 49) |

Subjects Randomized [N]

| | | |
|---|---|---|
| 53 (100.0) | 55 (100.0) | 49 (100.0) |

Subjects Included in Each Analysis Population [n (%)]

| | | | |
|---|---|---|---|
| Safety Population | 53 (100.0) | 55 (100.0) | 49 (100.0) |
| ITT Population | 53 (100.0) | 55 (100.0) | 49 (100.0) |
| Per-Protocol Population | 49 (92.5) | 50 (90.9) | 43 (87.8) |
| Pharmacokinetic | 51 (96.2) | 53 (96.4) | 48 (98.0) |
| Population | | | |

Subjects Completed the Study [n (%)] [2]

| | | | |
|---|---|---|---|
| Yes | 50 (94.3) | 52 (94.5) | 43 (87.8) |
| No | 3 (5.7) | 3 (5.5) | 6 (12.2) |

If No, Reason of Study Discontinuation [n (%)] [2]

| | | | |
|---|---|---|---|
| Adverse Event | 1 (1.9) | 1 (1.8) | 0 |
| Lost to follow-up | 1 (1.9) | 2 (3.6) | 0 |
| Withdrew consent | 0 | 0 | 6 (12.2) |
| Other | 1 (1.9) | 0 | 0 |

Subjects at Baseline [n (%)] [2]

| | | | |
|---|---|---|---|
| Safety Population | 53 (100.0) | 55 (100.0) | 49 (100.0) |
| ITT Population | 53 (100.0) | 55 (100.0) | 49 (100.0) |
| Per-Protocol Population | 49 (92.5) | 50 (90.9) | 43 (87.8) |
| Pharmacokinetic | 51 (96.2) | 53 (96.4) | 48 (98.0) |
| Population | | | |

[1] Percentages based on the number of subjects randomized.
[2] Percentages based on the number of subjects included in the safety population

TABLE 5
Subject Demographics

| | | |
|---|---|---|
| Vehicle | RVT-501 0.2% | RVT-501 0.5% |
| (N = 53 | (N = 55) | (N = 49) |

Age (years)

| | | | |
|---|---|---|---|
| N | 53 | 55 | 49 |
| Mean (SD) | 27.3 (14.24) | 28.0 (15.30) | 28.4 (16.35) |
| Median | 23.0 | 22.0 | 21.0 |
| Min, Max | 12, 58 | 12, 65 | 12, 67 |
| IQR | 15-42 | 16-39 | 16-39 |

Gender [n (%)]

| | | | |
|---|---|---|---|
| Male | 20 (37.7) | 23 (41.8) | 19 (38.8) |
| Female | 33 (62.3) | 32 (58.2) | 30 (61.2) |

Ethnicity [n (%)]

| | | | |
|---|---|---|---|
| Hispanic/Latino | 7 (13.2) | 3 (5.5) | 8 (16.3) |
| Not Hispanic/Latino | 46 (86.8) | 52 (94.5) | 41 (83.7) |

Race [n (%)]

| | | | |
|---|---|---|---|
| White | 30 (56.6) | 36 (65.5) | 25 (51.0) |
| Black or African | 13 (24.5) | 13 (23.6) | 16 (32.7) |
| American | | | |
| Native Hawaiian | 1 (1.9) | 0 | 0 |
| or Other | | | |
| Pacific Islander | | | |
| American Indian or | 0 | 0 | 1 (2.0) |
| Alaska Native | | | |
| Asian | 7 (13.2) | 5 (9.1) | 5 (10.2) |
| Other | 2 (3.8) | 1 (1.8) | 2 (4.1) |

Table 6 provides the summary of adverse events. Table 7 provides a summary of the adverse events by organ class.

TABLE 6
Summary of Treatment Emergent Adverse Events

| | Vehicle | RVT-501 0.2% | RVT-501 0.5% | Total |
|---|---|---|---|---|
| | (N = 53) | (N = 55) | (N = 49) | (N = 157) |
| Treatment Emergent | 17 | 20 | 25 | 62 |
| Adverse Events (TEAEs) | | | | |
| Reported [n] | | | | |
| Subjects With At Least One | 12 (22.6) | 14 (25.5) | 16 (32.7) | 42 (26.8) |
| TEAE [n (%)][1] | | | | |
| Subjects With At Least One | 3 (5.7) | 6 (109) | 4 (8.2) | 13 (8.3) |
| Drug-Related TEAE [n | | | | |
| (%)][1][3] | | | | |

Subject With At Least One TEAE for each Severity/Intensity [2]

| | | | | |
|---|---|---|---|---|
| Mild [n (%)] | 11 (64.7) | 11 (55.0) | 14 (56.0) | 36 (58.1) |
| Moderate [n (%)] | 6 (35.3) | 9 (45.0) | 11 (44.0) | 26 (41.9) |
| Severe [n (%)] | 0 | 0 | 0 | 0 |
| Life-threatening [n (%)] | 0 | 0 | 0 | 0 |
| Death [n (%)] | 0 | 0 | 0 | 0 |
| Subject With At Least One | 0 | 0 | 0 | 0 |
| Serious TEAE [n (%)][1] | | | | |
| Subject With At Least One | 0 | 0 | 0 | 0 |
| Drug-Related Serious TEAE | | | | |
| [n (%)][1] | | | | |

[1]Percentages are based on the number of subjects in the Safety population in each treatment group.
[2] Percentages are based on the total number of treatment emergent adverse events reported in each treatment group.
[3]AE that was reported as “related”
[4] When assessing adverse events, refer to the NIH Common Terminology Criteria for Adverse Events (CTCAE), v.4.02, 2009.

TABLE 7
Summary of Treatment Emergent Adverse Events by Organ Class

| | Vehicle | RVT-501 0.2% | RVT-501 0.5% | Total |
|---|---|---|---|---|
| | (N = 53) | (N = 55) | (N = 49) | (N = 157) |
| Subjects with at least one | 12 (22.6) | 14 (25.5) | 16 (32.7) | 42 (26.8) |
| TEAE [n (%)] | | | | |
| Blood and lymphatic system | 0 | 0 | 1 (2.0) | 1 (0.6) |
| disorders [n (%)] | | | | |
| Leukopenia [n (%)] | 0 | 0 | 1 (2.0) | 1 (0.6) |
| Ear and labyrinth disorders [n | 0 | 0 | 1 (2.0) | 1 (0.6) |
| (%)] | | | | |
| Tympanic membrane | 0 | 0 | 1 (2.0) | 1 (0.6) |
| perforation [n (%)] | | | | |
| Vertigo [n (%)] | 0 | 0 | 1 (2.0) | 1 (0.6) |
| Gastrointestinal disorders [n | 3 (5.7) | 1 (1.8) | 1 (2.0) | 5 (3.2) |
| (%)] | | | | |
| Dry mouth [n (%)] | 0 | 1 (1.8) | 0 | 1 (0.6) |
| Nausea [n (%)] | 2 (3.8) | 0 | 1 (2.0) | 3 (1.9) |
| Vomiting [n (%)] | 1 (1.9) | 0 | 1 (2.0) | 2 (1.3) |
| General disorders and | 4 (7.5) | 5 (9.1) | 3 (6.1) | 12 (7.6) |
| administration site conditions | | | | |
| [n (%)] | | | | |
| Application site pain [n (%)] | 2 (3.8) | 2 (3.6) | 1 (2.0) | 5 (3.2) |
| Application site pruritus [n | 1 (9) | 4 (7.3) | 2(4.1) | 7 (4.5) |
| (%)] | | | | |
| Influenza like illness [n (%)] | 1 (1.9) | 0 | 0 | 1 (0.6) |
| Infections and infestations [n | 5 (9.4) | 8 (14.5) | 9 (18.4) | 22 (14.0) |
| (%)] | | | | |
| Bronchitis [n (%)] | 0 | 1 (1.8) | 0 | 1 (0.6) |
| Gastroenteritis [n (%)] | 0 | 1 (1.8) | 0 | 1 (0.6) |
| Nasopharyngitis [n (%)] | 4 (7.5) | 4 (7.3) | 5 (10.2) | 13 (8.3) |
| Upper respiratory tract | 1 (1.9) | 3 (5.5) | 4 (8.2) | 8 (5.1) |
| infection [n (%)] | | | | |
| Vaginitis bacterial [n (%)] | 0 | 1 (1.8) | 0 | 1 (0.6) |
| Injury, poisoning and | 1 (1.9) | 0 | 0 | 1 (0.6) |
| procedural complications | | | | |
| [n (%)] | | | | |
| Laceration [n (%)] | 1 (1.9) | 0 | 0 | 1 (0.6) |
| Investigations [n (%)] | 1 (1.9) | 0 | 0 | 1 (0.6) |
| Hepatic enzyme increased [n | 1 (1.9) | 0 | 0 | 1 (0.6) |
| (%)] | | | | |
| Metabolism and nutrition | 0 | 0 | 1 (2.0) | 1 (0.6) |
| disorders [n (%)] | | | | |
| Type 2 diabetes mellitus [n | 0 | 0 | 1 (2.0) | 1 (0.6) |
| (%)] | | | | |
| Nervous System disorders [n | 1 (1.9) | 0 | 2 (4.1) | 3 (1.9) |
| (%)] | | | | |
| Dizziness [n (%)] | 1 (1.9) | 0 | 0 | 1 (0.6) |
| Headache [n (%)] | 1 (1.9) | 0 | 2 (4.1) | 3 (1.9) |
| Psychiatric disorders [n (%)] | 1 (1.9) | 0 | 0 | 1 (0.6) |
| Insomnia [n (%)] | 1 (1.9) | 0 | 0 | 1 (0.6) |
| Skin and subcutaneous tissue | 0 | 1 (1.8) | 5 (10.2) | 6 (3.8) |
| disorders [n (%)] | | | | |
| Dermatitis atopic [n (%)] | 0 | 0 | 3 (6.1) | 3 (1.9) |
| Photosensitivity reaction [n | 0 | 0 | 1 (2.0) | 1 (0.6) |
| (%)] | | | | |
| Skin burning sensation [n | 0 | 1 (1.8) | 0 | 1 (0.6) |
| (%)] | | | | |
| Skin exfoliation [n (%)] | 0 | 0 | 1 (2.0) | 1 (0.6) |

Note:
Each treatment emergent adverse event is counted only once for each subject within each System Organ Class and MedDRA Preferred Term.

Safety Results: RVT-501 0.2% and RVT-501 0.5% ointments were generally safe and well tolerated, and no serious adverse events (SAEs) nor deaths were reported during the study. Overall, 42 (26.8%) subjects experienced at least 1 TEAE during the study, with a total of 62 TEAEs reported. Twelve (22.6%) subjects experienced a TEAE in the vehicle group, 14 (25.5%) in the RVT-501 0.2% group, and 16 (32.7%) in the RVT-501 0.5% group. Most of the TEAEs were mild in intensity (58.1% of the reported TEAEs), 41.9% of the TEAEs were of moderate intensity, and none were severe or life-threatening. No subjects experienced a TEAE of grade 3 or higher. A similar frequency and severity of TEAEs was observed between treatment groups. The majority of TEAEs were considered unrelated to study drug. A total of 14 drug-related TEAEs were reported during the study.

One (1.9%) adult subject in the vehicle group (application site pain) and 1 (1.8%) adult subject in the RVT-501 0.2% group (application site pruritus and application site pain) reported TEAEs that led to study discontinuation.

The most common TEAEs across the treatment groups were those classified in the infections and infestations disorders. TEAEs that were reported by more than one subject were: nasopharyngitis (13 [8.3%] subjects), upper respiratory tract infection (8 [5.1%] subjects), application site pruritus (7 [4.5%] subjects), application site pain (5 [3.2%] subjects), nausea (3 [1.9%] subjects), dermatitis atopic (AD flare or worsening of eczema) (3 [1.9%] subjects), headache (3 [1.9%] subjects), and vomiting (2 [1.3%] subjects). A similar number of subjects experienced application site pain and pruritus across the treatment groups. No trends were detected between treatment groups, except that the 3 subjects (1.9%) who reported dermatitis atopic (AD flare or worsening of eczema) were in the RVT-501 0.5% group.

Proportionally, there was a higher percentage of patients in the adult population reporting TEAEs (34.7% in adults compared to 14.5% in adolescents) and drug-related TEAEs (11.6% in adults compared to 3.2% in adolescents) than in the adolescent population. Similarly, more TEAEs were mild in the adolescent population than in the adult population.

Three (1.91%) subjects (one adult in the vehicle group and 2 adults in the RTV-501 0.5% group) had a clinically significant finding for clinical biochemistry, hematology, or urinalysis results that resulted in TEAEs, and they were all considered unrelated to the study drug. No vital signs or ECG findings were considered to be clinically significant by the investigator during the study. Overall, there were no trends detected between treatment groups for the safety laboratory results, vital signs, and ECGs.

Pharmacokinetics summary: PK samples were collected pre-dose at Weeks 1 and 4, and 2-4 hrs post-dose at Week 4. Only 1 subject (an adolescent) had detectable RVT-501 above the LLQ (lower limit of quantitation, 1 ng/mL) at 1.23 ng/mL pre-dose and 2 hrs post-dose on Week 4. Three patients had detectable M11 exposure with the highest value at 1.60 ng/mL.

Pharmacokinetic Results: No measurable concentrations were reported for RVT-501 at Week 1 (pre-dose); values were below the LLQ (1.00 ng/mL) for all treatment groups. One adolescent subject in the RVT-501 0.2% group had measurable concentrations at Week 4, both pre-dose and post-dose values were near the LLQ (highest value was 1.23 ng/mL).

Plasma concentrations of M11 metabolite were measurable in 2 subjects at Week 1 (pre-dose) (1 adolescent subject in RVT-501 0.2% and 1 adult subject in RVT-501 0.5%) and in 1 adult subject at Week 4 (pre-dose) in the RVT-501 0.5% group. The highest concentration was 1.60 ng/mL and all concentrations were near the LLQ (1.00 ng/mL). The data demonstrate minimal to no systemic absorption of RVT-501 or its active metabolite.

Measurable concentrations of plasma RVT-501 were reported in the RVT-501 0.2% group in 1 adolescent subject (Subject 18014) at Week 4, pre-dose and 2 hours post-dose (1.23 and 1.20 ng/mL, respectively). This subject had an IGA score of 3 (moderate), a total EASI score of 7.8, and a BSA affected by AD of 9% at baseline. Measurable concentrations of plasma M11 were reported pre-dose in the RVT 501 0.2% group in 1 adolescent subject (Subject 18014) at Week 1 (1.27 ng/mL) and in the RVT-501 0.5% group in 2 adult subjects (Subject 03005 and Subject 09003), respectively at Week 1 (1.60 ng/mL) and at Week 4 (1.09 ng/mL). These subjects had an IGA score of 3 (moderate), a total EASI score of 26.1 and 20.0, and a BSA affected by AD of 35% and 17% at baseline, respectively.

Efficacy Results, see Table 8.

TABLE 8
Results

| | | | | |
|---|---|---|---|---|
| | | Vehicle | RVT-501 0.2% | RVT-501 0.5% |
| Age Group | Statistics | (N = 53) | (N = 55) | (N = 49) |

Proportion of Subjects with at Least a 2-point Reduction in IGA to Clear or Almost Clear at Week 4

| | | | | |
|---|---|---|---|---|
| Adult | N (%) | 6 (19.4) | 8 (23.5) | 6 (20.0) |
| Adolescent | N (%) | 2 (9.1) | 4 (19.0) | 6 (31.6) |
| Overall | N (%) | 8 (15.1) | 12 (21.8) | 12 (24.5) |

Proportion of Subjects Who Achieved an IGA Score of Clear or Almost Clear at Week 4

| | | | | |
|---|---|---|---|---|
| Adult | N (%) | 6 (19.4) | 10 (29.4) | 6 (20.0) |
| Adolescent | N (%) | 3 (13.6) | 5 (23.8) | 7 (36.8) |
| Overall | N (%) | 9 (17.0) | 15 (27.3) | 13 (26.5) |

Mean Percent Change from Baseline in BSA at Week 4

| | | | | |
|---|---|---|---|---|
| Adult | Mean (SD) | −37.5 (37.72) | −44.3 (33.39) | −44.5 (35.47) |
| Adolescent | Mean (SD) | −31.4 (39.11) | −49.5 (33.05) | −40.9 (38.67) |
| Overall | Mean (SD) | −35.0 (38.05) | −46.2 (33.05) | −43.1 (36.38) |

Mean Percent Change from Baseline in EASI Scores at Week 4

| | | | | |
|---|---|---|---|---|
| Adult | Mean (SD) | −50.7 (36.07) | −56.1 (30.42) | −53.4 (39.78) |
| Adolescent | Mean (SD) | −49.1 (35.18) | −61.8 (23.55) | −56.0 (39.75) |
| Overall | Mean (SD) | −50.0 (35.37) | −58.3 (27.97) | −54.4 (39.38) |

Mean Percent Change from Baseline in Pruritus NRS at Week 4

| | | | | |
|---|---|---|---|---|
| Adult | Mean (SD) | −27.5 (53.92) | −39.2 (46.83) | −38.6 (45.09) |
| Adolescent | Mean (SD) | −37.8 (44.44) | −19.8 (77.48) | −31.6 (49.17) |
| Overall | Mean (SD) | −29.3 (49.72) | −31.5 (60.90) | −35.9 (46.29) |

BSA = Body Surface Area; EASI: Eczema Area and Severity Index; IGA = Investigator's Global Assessment; NRS = Numeric Rating Scale; SD = standard deviation.

Over time, there was an incremental increase in the proportion of subjects presenting an improvement in IGA scores in each treatment group. For RVT-501 0.2%, the increase was more pronounced compared with the vehicle from Day 4 to Week 1, and similar results were observed starting from Week 2. For RVT-501 0.5%, the increase was more pronounced compared with the vehicle at Day 4 and Weeks 1 and 4.

As shown in Table 9, overall, at Week 4, a total of 102 subjects out of 157 (65.0%) had an improvement in their IGA score. This included 34 of 53 [64.1%] in the vehicle group, 35 of 55 [63.6%] in the RVT-501 0.2% group, and 33 of 49 [67.3%] in the RVT 501 0.5% group. Three (3) subjects (1.9%) had a worsening in their IGA scores (1 subject [1.9%] in the vehicle group and 2 subjects [4.1%] in the RVT-501 0.5% group).

TABLE 9
Shift Table from Baseline for IGA at Week 4 - Overall Age Group (ITT Population)

| | Week 4 Values, N (%) | | | | | |
|---|---|---|---|---|---|---|
| | | Almost | Mild | Moderate | | |
| | Clear | Clear | Disease | Disease | Severe | |
| | (0) | (1) | (2) | (3) | (4) | Total |
| Baseline Values, N (%) | | | | | | |
| Vehicle (N = 53) | | | | | | |
| Clear (0) | 0 | 0 | 0 | 0 | 0 | 0 |
| Almost Clear (1) | 0 | 0 | 0 | 0 | 0 | 0 |
| Mild Disease (2) | 0 | 1 (1.9) | 2 (3.8) | 1 (1.9) | 0 | 4 (7.5) |
| Moderate Disease (3) | 1 (1.9) | 7 (13.2) | 25 (47.2) | 16 (30.2) | 0 | 49 (92.5) |
| Severe Disease (4) | 0 | 0 | 0 | 0 | 0 | 0 |
| Total | 1 (1.9) | 8 (15.1) | 27 (50.9) | 17 (32.1) | 0 | 53 (100) |
| Baseline Values, N (%) | | | | | | |
| RVT-501 0.2% (N = 55) | | | | | | |
| Clear (0) | 0 | 0 | 0 | 0 | 0 | 0 |
| Almost Clear (1) | 0 | 0 | 0 | 0 | 0 | 0 |
| Mild Disease (2) | 1 (1.8) | 3 (5.5) | 4 (7.3) | 0 | 0 | 8 (14.5) |
| Moderate Disease (3) | 1 (1.8) | 10 (18.2) | 20 (36.4) | 16 (29.1) | 0 | 47 (85.5) |
| Severe Disease (4) | 0 | 0 | 0 | 0 | 0 | 0 |
| Total | 2 (3.6) | 13 (23.6) | 24 (43.6) | 16 (29.1) | 0 | 55 (100) |
| Baseline Values, N (%) | | | | | | |
| RVT-501 0.5% (N = 49) | | | | | | |
| Clear (0) | 0 | 0 | 0 | 0 | 0 | 0 |
| Almost Clear (1) | 0 | 0 | 0 | 0 | 0 | 0 |
| Mild Disease (2) | 2 (4.1) | 1 (2.0) | 2 (4.1) | 0 | 0 | 5 (10.2) |
| Moderate Disease (3) | 1 (2.0) | 9 (18.4) | 20 (40.8) | 12 (24.5) | 2 (4.1) | 44 (89.8) |
| Severe Disease (4) | 0 | 0 | 0 | 0 | 0 | 0 |
| Total | 3 (6.1) | 10 (20.4) | 22 (44.9) | 12 (24.5) | 2 (4.1) | 49 (100) |

Proportion of Subjects Who Achieved a Clear or Almost Clear with at Least a Decrease of 2 Points in Investigator's Global Assessment: The proportion of subjects who achieved an IGA of 0 (clear) or 1 (almost clear) and had at least a 2-point reduction from baseline (i.e., responders) is presented in Table 14.2.2.2.1 for the ITT population. Table 10 summarizes the proportion of subjects in each age group with at least a 2-point reduction attaining clear or almost clear at Week 4 for the ITT population. Overall, the number of responders was numerically higher for both RVT-501 0.2% and RVT-501 0.5% compared with the vehicle.

As shown in Table 10, at Week 4, the difference in IGA responders was even more pronounced for the adolescents, especially when comparing RVT-501 0.5% with the vehicle (adolescent: 2 [9.1%] responders in the vehicle group, 4 [19.0%] responders in the RVT-501 0.2% group, and 6 [31.6%] responders in the RVT-501 0.5% group; 95% CI: 10.4-31.4).

TABLE 10
Proportion of Subjects Who Achieved a Clear or Almost Clear with
at Least a Decrease of 2 Points in IGA Over Time (ITT Population)

| | Vehicle | RVT-501 0.2% | RVT-501 0.5% | |
|---|---|---|---|---|
| Age Group | (N = 53) | (N = 55) | (N = 49) | 95% CI |
| Adult, N (%) | 6 (19.4) | 8 (23.5) | 6 (20.0) | 13.4-30.6 |
| Adolescent, | 2 (9.1) | 4 (19.0) | 6 (31.6) | 10.4-31.4 |
| N (%) | | | | |
| Overall, N (%) | 8 (15.1) | 12 (21.8) | 12 (24.5) | 14.4-27.5 |

CI = confidence interval
Note:
95% CI was obtained from an exact binomial test.
95% CI was computed for all three treatment groups combined.

Proportion of Subjects Who Achieved an Investigator's Global Assessment of clear or almost clear: Table 11 summarizes the proportion of subjects in each age group attaining clear or almost clear at Week 4 for the ITT population. Similarly to that observed for the proportion of subjects who achieved an IGA of 0 or 1 with at least a decrease of 2 points in IGA, the proportion of subjects who achieved an IGA of 0 or 1 increased in each treatment group until Week 4. Overall, the number of responders was numerically higher for both RVT-501 0.2% and RVT-501 0.5% compared with the vehicle. As shown in Table 11, at Week 4, the difference in responders was even more pronounced for the adolescents, especially when comparing RVT-501 0.5% with the vehicle (adolescent: 3 [13.6%] responders in the vehicle group, 5 [23.8%] responders in the RVT-501 0.2% group, 7 [36.8] responders in the RVT-501 0.5% group; CI: 14.2-36.7).

TABLE 11
Proportion of Subjects Who Achieved an IGA Score of
Clear or Almost Clear at Week 4 (ITT Population)

| | Vehicle | RVT-501 0.2% | RVT-501 0.5% | |
|---|---|---|---|---|
| Age Group | (N = 53) | (N = 55) | (N = 49) | 95% CI |
| Adult, N (%) | 6 (19.4) | 10 (29.4) | 6 (20.0) | 15.1-32.9 |
| Adolescent, | 3 (13.6) | 5 (23.8) | 7 (36.8) | 14.2-36.7 |
| N (%) | | | | |
| Overall, N (%) | 9 (17.0) | 15 (27.3) | 13 (26.5) | 17.2-31.0 |

CI = confidence interval
Note:
95% CI was obtained from an exact binomial test.
95% CI was computed for all three treatment groups combined.

Change from Baseline in Body Surface Area Affected: Table 12 summarizes the percent change from baseline at Week 4 for the ITT population. Overall, the results were numerically higher for both RVT-501 0.2% and RVT-501 0.5% compared with the vehicle. As shown in Table 12, at Week 4, the difference in percent change from baseline was more pronounced for the adolescents when comparing RVT-501 0.2% with the vehicle (mean percent change from baseline [SD] for the adolescent group: −31.4% [39.11%] in the vehicle, −49.5% [33.05%] in the RVT-501 0.2% group, and −40.9% [38.67%] in the RVT-501 0.5% group).

TABLE 12
Summary of Percent Change from Baseline in BSA at Week 4 (ITT Population)

| | | Vehicle | RVT-501 0.2% | RVT-501 0.5% |
|---|---|---|---|---|
| Age Group | Statistics | (N = 53) | (N = 55) | (N = 49) |
| Adult | N | 31 | 34 | 30 |
| | Mean (SD) | −37.5 (37.72) | −44.3 (33.39) | −44.5 (35.47) |
| | Median | −42.4 | −41.5 | −50.6 |
| | Min, Max | −100.0, 73.3 | −100.0, 3.0 | −100.0, 44.4 |
| | IQR | −61.5-−14.3 | −75.0-−10.7 | −66.7-−32.2 |
| Adolescent | N | 22 | 21 | 19 |
| | Mean (SD) | −31.4 (39.11) | −49.5 (33.05) | −40.9 (38.67) |
| | Median | −41.5 | −57.4 | −44.4 |
| | Min, Max | −95.8, 50.0 | −100.0, 20.0 | −100.0, 48.0 |
| | IQR | −62.5-0.0 | −68.2-−20.0 | −76.9-0.0 |
| Overall | N | 53 | 55 | 49 |
| | Mean (SD) | −35.0 (38.05) | −46.2 (33.06) | −43.1 (36.38) |
| | Median | −42.1 | −50.0 | −45.5 |
| | Min, Max | −100.0, 73.3 | −100.0, 20.0 | −100.0, 48.0 |
| | IQR | −62.5-−6.5 | −75.0-−15.8 | −66.7-−25.0 |

IQR = interquartile range; SD = standard deviation

Change from Baseline in Eczema Area and Severity Index Score: Table 13 summarizes the percent change from baseline at Week 4 for the ITT population. Overall, the results were numerically higher for both RVT-501 0.2% and RVT-501 0.5% compared with the vehicle, although no statistically significant differences were noted among treatment groups (p-value: >0.05). RVT-501 0.2% and RVT-501 0.5% as well as the vehicle showed high responses over time in improvement of EASI.

As shown in Table 13, at Week 4, the differences in percent change from baseline were more pronounced for the adolescents, especially when comparing RVT-501 0.2% with the vehicle (mean [SD] for the adolescent group: −49.1% [35.18%] in the vehicle group, −61.8% [23.55%] in the RVT 501 0.2% group, and −56.0% [39.750%] in the RVT 501 0.5% group).

TABLE 13
Summary of Percent Change from Baseline in EASI Scores at Week 4 (ITT Population)

| | | Vehicle | RVT-501 0.2% | RVT-501 0.5% |
|---|---|---|---|---|
| Age Group | Statistics | (N = 53) | (N = 55) | (N = 49) |
| Adult | N | 31 | 34 | 30 |
| | Mean (SD) | −50.7 (36.07) | −56.1 (30.42) | −53.4 (39.78) |
| | Median | −54.1 | −57.8 | −61.9 |
| | Min, Max | −100.0, 26.9 | −100.0, 29.6 | −100.0, 37.7 |
| | IQR | −82.3-−25.0 | −81.6-−33.3 | −85.0-−38.2 |
| | LS Means (SE) | −49.9 (6.36) | −56.4 (6.05) | −53.9 (6.45) |
| | 95% CI | −62.5, −37.2 | −68.4, −44.4 | −66.7, −41.0 |
| | 95% CI (Difference A vs C or B vs C) | | −26.3, 13.2 | −24.4, 16.4 |
| | p-value | | 0.677 | 0.869 |
| Adolescent | N | 22 | 21 | 19 |
| | Mean (SD) | −49.1 (35.18) | −61.8 (23.55) | −56.0 (39.75) |
| | Median | −62.0 | −64.5 | −67.9 |
| | Min, Max | −86.3, 11.6 | −100.0, −5.1 | −100.0, 39.5 |
| | IQR | −76.3-−17.5 | −77.8-−44.7 | −90.8-−34.6 |
| | LS Means (SE) | −51.1 (7.08) | −61.9 (7.15) | −53.6 (7.64) |
| | 95% CI | −65.3, −37.0 | −76.2, −47.6 | −68.9, −38.3 |
| | 95% CI (Difference A vs C or B vs C) | | −33.6, 12.0 | −26.4, 21.5 |
| | p-value | | 0.461 | 0.961 |
| Overall | N | 53 | 55 | 49 |
| | Mean (SD) | −50.0 (35.37) | −58.3 (27.91) | −54.4 (39.38) |
| | Median | −56.9 | −62.3 | −64.3 |
| | Min, Max | −100.0, 26.9 | −100.0, 29.6 | −100.0, 39.5 |
| | IQR | −78.7-−25.0 | −81.1-−41.3 | −87.1-−36.3 |
| | LS Means (SE) | −50.3 (4.66) | −58.5 (4.57) | −53.8 (4.85) |
| | 95% CI | −59.5, −41.1 | −67.5, −49.5 | −63.4, −44.3 |
| | 95% CI (Difference A vs C or B vs C) | | −22.8, 6.4 | −18.6, 11.5 |
| | p-value | | 0.351 | 0.822 |

CI = confidence interval; IQR = interquartile range; LS = least squares; SE = standard error
Notes:
A = RVT-501 0.2%; B = RVT-501 0.5%; C = Vehicle.
P-values, LS Means, and 95% CIs were obtained from an ANCOVA model.

EASI-50 Analysis: Table 14 summarizes the proportion of subjects achieving at least 50% reduction in EASI at Week 4 for the ITT population. Over time, for each treatment group, there was an increase in EASI-50 from Day 4 to Week 4. Overall, the results were numerically higher for both RVT-501 0.2% and RVT-501 0.5% compared with the vehicle. Similarly to the changes from baseline, it was observed that RVT 501 0.2% and RVT-501 0.5% as well as the vehicle showed high responses over time.

As shown in Table 14, at Week 4, similar results were observed among age groups. Nevertheless, the EASI 50 was slightly higher in the RVT-501 0.2% group and in the RVT 501 0.5% group than in the vehicle group (overall: 30 [56.6%] subjects in the vehicle group, 36 [65.5%] subjects in the RVT 501 0.2% group, and 33 [67.3%] subjects in the RVT-501 0.5% group).

TABLE 14
Proportion of Subjects Achieving at Least 50%
Reduction in EASI at Week 4 (ITT Population)

| | Vehicle | RVT-501 0.2% | RVT-501 0.5% |
|---|---|---|---|
| Age Group | (N = 53) | (N = 55) | (N = 49) |
| Adult, N (%) | 16 (51.6) | 22 (64.7) | 20 (66.7) |
| Adolescent, N (%) | 14 (63.6) | 14 (66.7) | 13 (68.4) |
| Overall, N (%) | 30 (56.6) | 36 (65.5) | 33 (67.3) |

Change from Baseline in Pruritus as Measured with the Numeric Rating Scale: The pruritus NRS asks the subject to rate the current severity of his/her itch from “no itch (0),” to “worst imaginable itch (10)”. Table 15 summarizes the percent change from baseline in pruritus NRS at Week 4 for the ITT population. Overall, the results were numerically superior (lower pruritus) for RVT-501 0.5% compared with the vehicle.

As shown in Table 15, at Week 4, the percent change from baseline was numerically higher (lower pruritus) for both RVT-501 0.2% and RVT-501 0.5% compared with the vehicle in the adult group (mean [SD] for the adult age group: −27.5% [53.92%] in the vehicle group, −39.2% [46.83%] in the RVT 501 0.2% group, and −38.6% [45.09%] in the RVT-501 0.5% group). There was no decrease in pruritus NRS in the adolescent group.

TABLE 15
Summary of Percent Change from Baseline
in Pruritus at Week 4 (ITT Population)

| | | Vehicle | RVT-501 0.2% | RVT-501 0.5% |
|---|---|---|---|---|
| Age Group | Statistics | (N = 53) | (N = 55) | (N = 49) |
| Adult | N | 29 | 32 | 29 |
| | Mean (SD) | −27.5 (53.92) | −39.2 (46.83) | −38.6 (45.09) |
| | Median | −33.3 | −45.0 | −37.5 |
| | Min, Max | −87.5, 166.7 | −100.0, 50.0 | −100.0, 42.9 |
| | IQR | −71.4-0.0 | −75.7-0.0 | −77.8-0.0 |
| Adolescent | N | 21 | 21 | 18 |
| | Mean (SD) | −31.8 (44.44) | −19.8 (77.48) | −31.6 (49.17) |
| | Median | −37.5 | −28.6 | −32.5 |
| | Min, Max | −100.0, 100.0 | −100.0, 250.0 | −100.0, 50.0 |
| | IQR | −50.0-0.0 | −75.0-0.0 | −62.5-0.0 |
| Overall | N | 50 | 53 | 47 |
| | Mean (SD) | −29.3 (49.72) | −31.5 (60.90) | −35.9 (46.29) |
| | Median | −35.4 | −40.0 | −37.5 |
| | Min, Max | −100.0, 166.7 | −100.0, 250.0 | −100.0, 50.0 |
| | IQR | −66.7-0.0 | −75.0-0.0 | −77.8-0.0 |

IQR = interquartile range; SD = standard deviation

Patient-Reported Symptoms: Table 16 shows shift from baseline for patient-reported symptoms at Week 4 for the overall age group, for the ITT population. At Week 4, a total of 58 (36.9%) subjects reported an improvement in their burning sensation. This included 18 of 53 [34.0%] in the vehicle group, 22 of 55 [40.0%] in the RVT-501 0.2% group, and 18 of 49 [36.7%] in the RVT-501 0.5% group). Seventy-seven (77) (49.0%) subjects reported an improvement in their pruritus (23 of 53 [43.4%] in the vehicle group, 27 of 55 [49.1%] in the RVT-501 0.2% group, and 27 of 49 [55.1%] in the RVT-501 0.5% group) compared with baseline. As previously observed, adults reported an improvement of their symptoms after application of both RVT-501 0.2% and RVT-501 0.5% compared with vehicle, but this was not observed in the adolescent group.

TABLE 16
Shift from Baseline for Patient-Reported Symptoms
at Week 4 - Overall Age Group (ITT Population)

| Week 4 Values, N (%) | | | | |
|---|---|---|---|---|
| None (0) | Mild (1) | Moderate (2) | Severe (3) | Total (4) |

BURNING

| | | | | | |
|---|---|---|---|---|---|
| Baseline Values, N (%) | | | | | |
| Vehicle (N = 53) | | | | | |
| None (0) | 7 (13.2) | 4 (7.5) | 2 (3.8) | 0 | 13 (24.5) |
| Mild (1) | 11 (20.8) | 8 (15.1) | 3 (5.7) | 1 (1.9) | 23 (43.4) |
| Moderate (2) | 3 (5.7) | 4 (7.5) | 7 (13.2) | 0 | 14 (26.4) |
| Severe (3) | 0 | 1 (1.9) | 0 | 1 (1.9) | 2 (3.8) |
| Total | 21 (39.6) | 17 (32.1) | 12 (22.6) | 2 (3.8) | 52 (98.1) |
| Baseline Values, N (%) | | | | | |
| RVT-501 0.2% (N = 55) | | | | | |
| None (0) | 14 (25.5) | 8 (14.5) | 2 (3.6) | 0 | 24 (43.6) |
| Mild (1) | 12 (21.8) | 3 (5.5) | 1 (1.8) | 0 | 16 (29.1) |
| Moderate (2) | 10 (18.2) | 0 | 3 (5.5) | 0 | 13 (23.6) |
| Severe (3) | 0 | 0 | 0 | 0 | 0 |
| Total | 36 (65.5) | 11 (20.0) | 6 (10.9) | 0 | 53 (96.4) |
| Baseline Values, N (%) | | | | | |
| RVT-501 0.5% (N = 49) | | | | | |
| None (0) | 13 (26.5) | 5 (10.2) | 1 (2.0) | 1 (2.0) | 20 (40.8) |
| Mild (1) | 8 (16.3) | 8 (16.3) | 2 (4.1) | 0 | 18 (36.7) |
| Moderate (2) | 2 (4.1) | 2 (4.1) | 0 | 0 | 4 (8.2) |
| Severe (3) | 2 (4.1) | 4 (8.2) | 0 | 1 (2.0) | 7 (14.3) |
| Total | 25 (51.0) | 19 (38.8) | 3 (6.1) | 2 (4.1) | 49 (100.0) |

PRURITUS

| | | | | | |
|---|---|---|---|---|---|
| Baseline Values, N (%) | | | | | |
| Vehicle (N = 53) | | | | | |
| None (0) | 0 | 3 (5.7) | 1 (1.9) | 0 | 4 (7.5) |
| Mild (1) | 1 (1.9) | 4 (7.5) | 3 (5.7) | 2 (3.8) | 10 (18.9) |
| Moderate (2) | 3 (5.7) | 13 (24.5) | 11 (20.8) | 3 (5.7) | 30 (56.6) |
| Severe (3) | 0 | 0 | 6 (11.3) | 2 (3.8) | 8 (15.1) |
| Total | 4 (7.5) | 20 (37.7) | 21 (39.6) | 7 (13.2) | 52 (98.1) |
| Baseline Values, N (%) | | | | | |
| RVT-501 0.2% (N = 55) | | | | | |
| None (0) | 2 (3.6) | 1 (1.8) | 1 (1.8) | 0 | 4 (7.3) |
| Mild (1) | 4 (7.3) | 4 (7.3) | 4 (7.3) | 1 (1.8) | 13 (23.6) |
| Moderate (2) | 4 (7.3) | 10 (18.2) | 10 (18.2) | 1 (1.8) | 25 (45.5) |
| Severe (3) | 1 (1.8) | 4 (7.3) | 4 (7.3) | 2 (3.6) | 11 (20.0) |
| Total | 11 (20.0) | 19 (34.5) | 19 (34.5) | 4 (7.3) | 53 (96.4) |
| Baseline Values, N (%) | | | | | |
| RVT-501 0.5% (N = 49) | | | | | |
| None (0) | 2 (4.1) | 0 | 0 | 2 (4.1) | 4 (8.2) |
| Mild (1) | 3 (6.1) | 6 (12.2) | 2 (4.1) | 1 (2.0) | 12 (24.5) |
| Moderate (2) | 5 (10.2) | 8 (16.3) | 7 (14.3) | 1 (2.0) | 21 (42.9) |
| Severe (3) | 2 (4.1) | 2 (4.1) | 7 (14.3) | 1 (2.0) | 12 (24.5) |
| Total | 12 (24.5) | 16 (32.7) | 16 (32.7) | 5 (10.2) | 49 (100.0) |

If there were no subjects with baseline patient reported symptoms PRS of none (0), then that row was deleted.

Change from Baseline in Patient-Oriented Eczema Measure: Table 17 shows the percent change from baseline in POEM at Week 4 for the ITT population. At Week 4, the subjects reported an improvement of the severity of their condition in the three treatment groups. Overall, the results were numerically higher for both RVT-501 0.2% and RVT 501 0.5% compared with the vehicle and the difference was more pronounced for RVT 501 0.5% (mean percent change from baseline [SD] overall: 32.9% [33.87%] in the vehicle, −36.4% [44.30%] in the RVT-501 0.2% group, and −40.8% [39.09%] in the RVT-501 0.5% group).

TABLE 17
Summary of Percent Change from Baseline in POEM at Week 4 (ITT Population)

| | | Vehicle | RVT-501 0.2% | RVT-501 0.5% |
|---|---|---|---|---|
| Age Group | Statistics | (N = 53) | (N = 55) | (N = 49) |
| Adult | N | 30 | 32 | 30 |
| | Mean (SD) | −33.0 (36.10) | −33.5 (50.88) | −45.1 (35.15) |
| | Median | −34.6 | −39.8 | −52.1 |
| | Min, Max | −80.0, 62.5 | −100.0, 166.7 | −100.0, 21.1 |
| | IQR | −65.4-−8.3 | −69.1-−6.0 | −66.7-−8.7 |
| Adolescent | N | 22 | 21 | 19 |
| | Mean (SD) | −32.7 (31.40) | −40.9 (32.52) | −34.1 (44.80) |
| | Median | −31.0 | −41.7 | −46.4 |
| | Min, Max | −87.5, 28.6 | −100.0, 33.3 | −100.0, 83.3 |
| | IQR | −60.9-−4.0 | − 62.5-−25.0 | −68.2-0.0 |
| Overall | N | 52 | 53 | 49 |
| | Mean (SD) | −32.9 (33.87) | −36.4 (44.30) | −40.8 (39.09) |
| | Median | −32.5 | −41.7 | −50.0 |
| | Min, Max | −87.5, 62.5 | −100.0, 166.7 | −100.0, 83.3 |
| | IQR | −62.9-−6.4 | −66.7-−8.7 | −66.7-−4.5 |

IQR = interquartile range; SD = standard deviation

Patient Diary: The following signs and symptoms were self-assessed over time in the adult and adolescent age groups: itchy skin, red or discolored skin, bleeding skin, oozing skin, cracked skin, scaly, flaky skin; dry or rough skin, painful, and burning or stinging skin. In the adult group, at Week 4, an improvement in these signs and symptoms was reported by the subjects in all treatment groups, with the exception of painful, and burning or stinging skin that the adult subjects reported as worsened in the vehicle group. For both RVT-501 0.2% and RVT 501 0.5%, a numerically higher improvement in these signs and symptoms was reported compared with the vehicle and no clear difference was observed among the active treatment groups. Similar results were observed in the adolescent population, except that the subjects reported a higher improvement in the RVT-501 0.2% group than in the RVT-501 0.5% group.

As a secondary objective, the efficacy assessments showed similar results in both the ITT and PP populations:

A higher percentage of subjects in the RVT-501 treatment groups achieved an IGA score of 0 or 1 with a 2-point improvement from baseline. Overall, endpoints involving IGA showed a numerically higher number of responders for both RVT-501 0.2% and RVT-501 0.5% compared with the vehicle. RVT-501 generally demonstrated a rapid and dose-dependent response compared to vehicle during the first 2 weeks of treatment. The differential response between treatment groups diminished starting at Week 3 due to increases in response in the vehicle group. Adolescent subjects treated with RVT-501 displayed greater treatment effects than adults.

Similar results were observed for endpoints involving BSA and EASI. There was a decrease in the percentage of affected BSA and an improvement in EASI scores across the treatment groups, which were generally numerically higher for both active treatments compared with the vehicle. These parameters showed a discrete separation between arms and age groups at Week 4. BSA and EASI results also showed a rapid response from Week 0 to Week 2 in the RVT-501 treatment groups, with an increased vehicle response starting at Week 3. Adolescents displayed more pronounced results, especially when comparing RVT-501 0.2% with the vehicle. However, EASI-50 results showed a higher response for RVT-501 0.5%.

Results from the pruritus NRS, the patient-reported symptoms of burning and pruritus, and the POEM show improvement in the adult group for both tested concentrations, although a high response also occurred in the vehicle group. The results were less clear in the adolescent group. Pruritus NRS showed a numerical decrease in the RVT-501 0.5% group as early as Week 1. An increased vehicle response was also seen as early as Week 2.

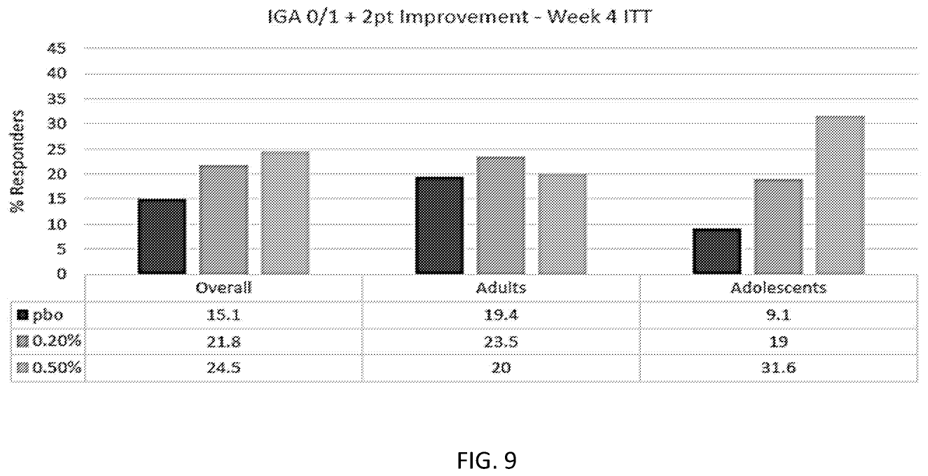

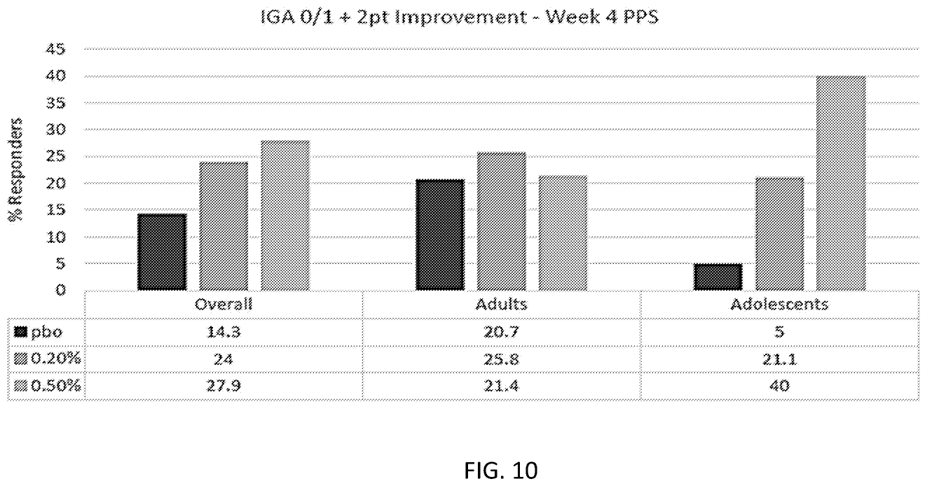

FIG. 9 provides the response in IGA (0/1+2 point improvement) at week 4 in the ITT population. FIG. 10 provides the response in IGA (0/1+2 point improvement) at week 4 in the PPS population. Adolescents responded better than adults in both populations.

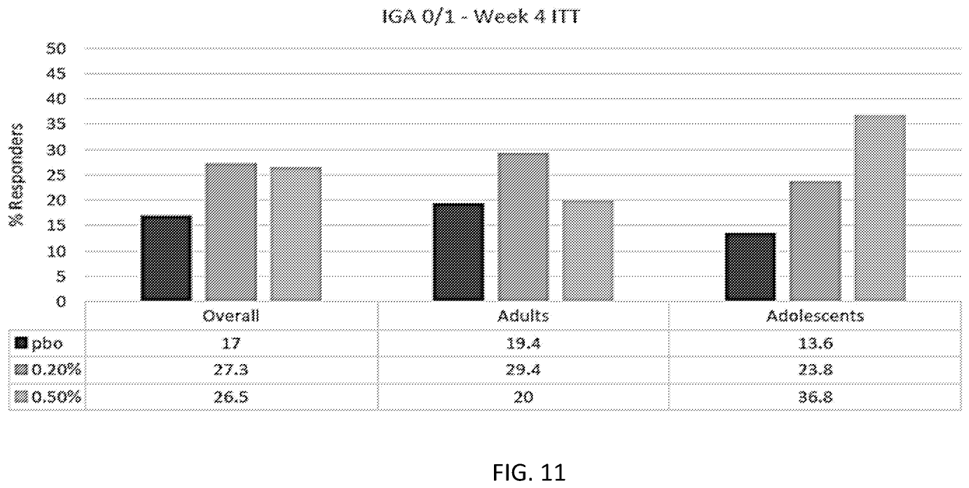

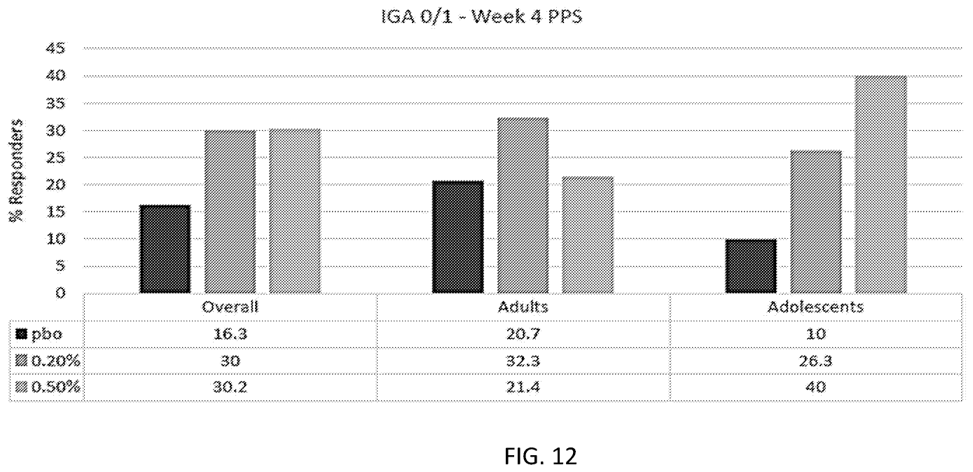

FIG. 11 provides the response in IGA (0/1) at week 4 in the ITT population. FIG. 12 provides the response in IGA (0/1) at week 4 in the PPS population. Adolescents responded better than adults in both populations.

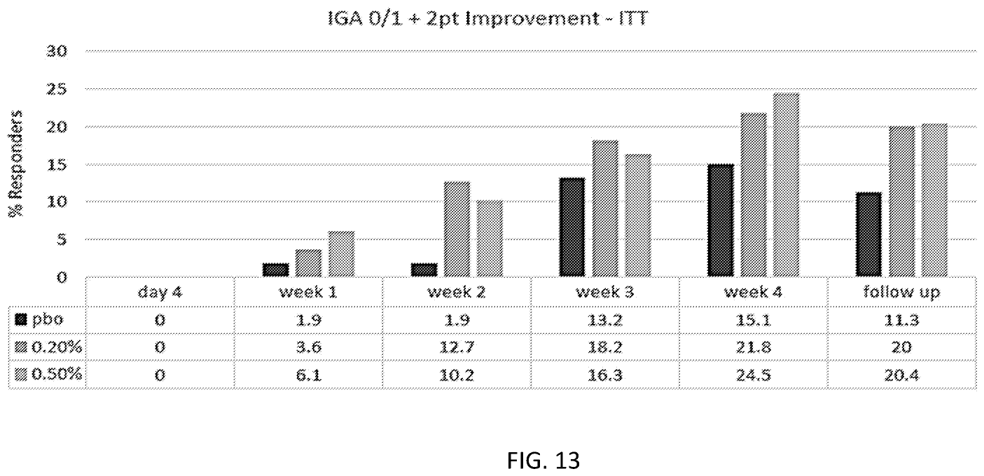

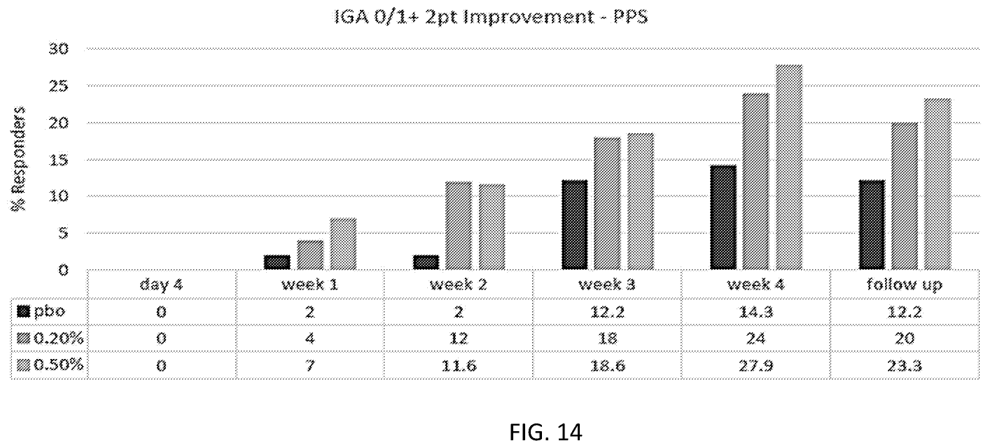

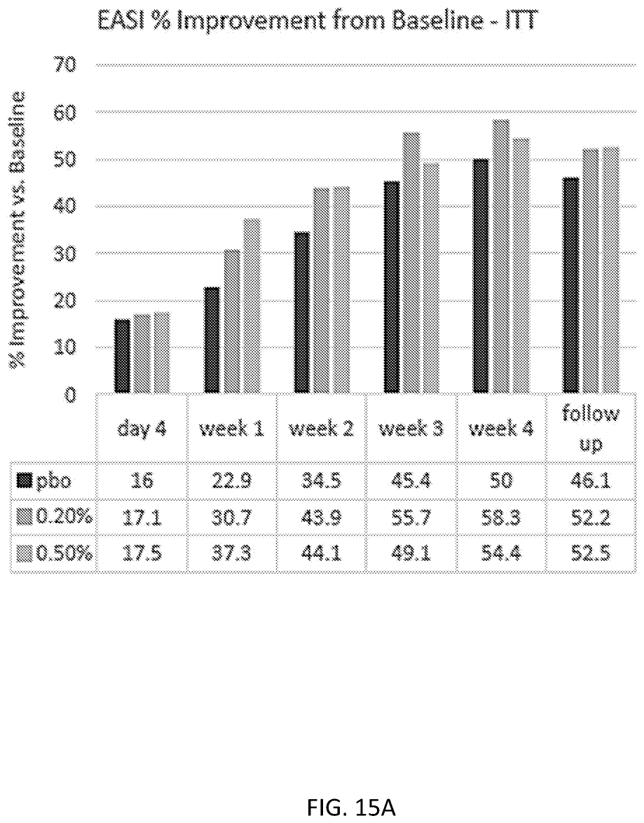

FIG. 13 provides the IGA response (0/1+2 point improvement) kinetics in the ITT population. FIG. 14 provides the IGA response (0/1+2 point improvement) kinetics in the PPS population. Rapid vehicle response was observed after 2 weeks of treatment. Both ITT and PPS populations exhibit similar time-course curves.

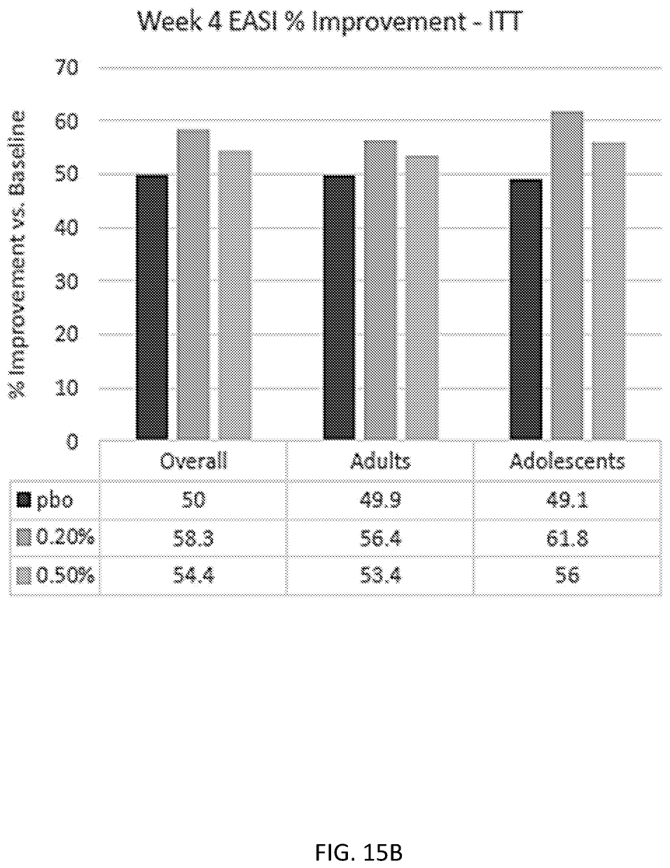

FIG. 15 shows the EASI % improvement from baseline and the week 4 EASI % improvement in the ITT population. RVT-501 exhibited high vehicle response in improvement in EASI. Minimal separation between arms and age groups at Week 4. Faster response observed in active arms vs. vehicle.

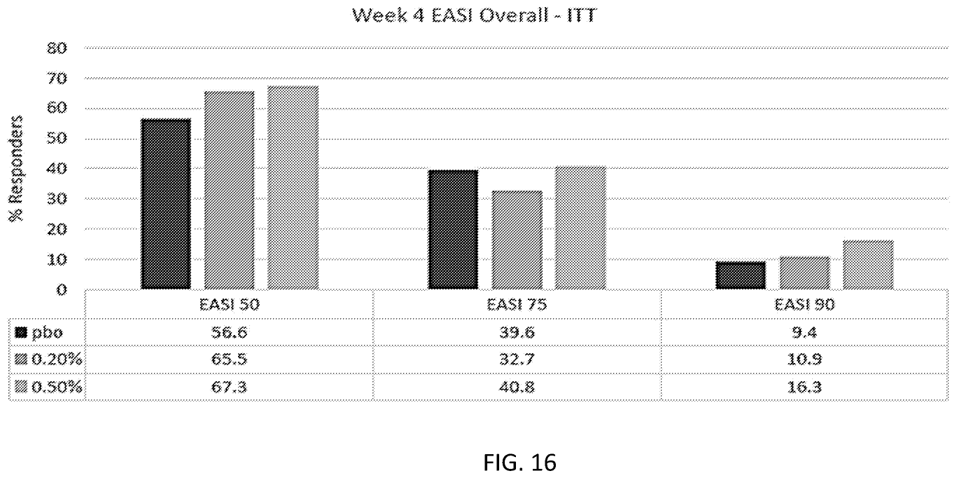

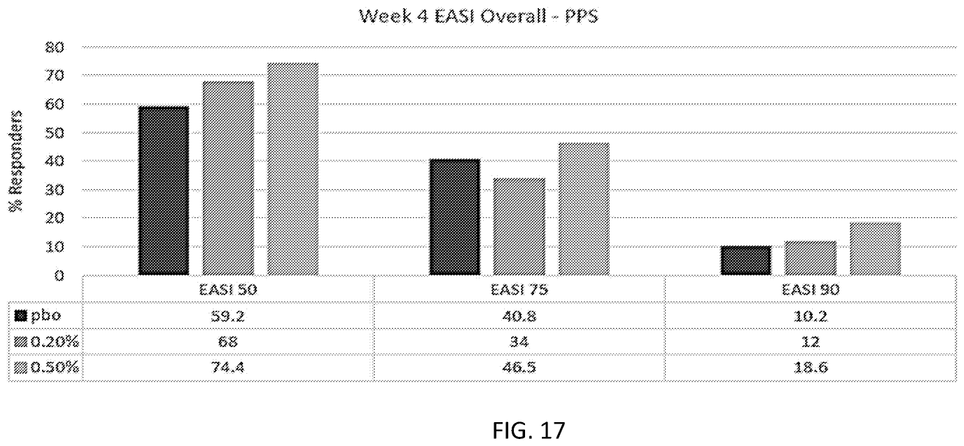

FIG. 16 provides data of EASI 50/75/90 responders at week 4 for the ITT population. FIG. 17 provides data of EASI 50/75/90 responders at week 4 for the PPS population. Separation was observed in active arms vs. vehicle for EASI50 and EASI 90. Very high vehicle response was observed in ITT and PPS populations.

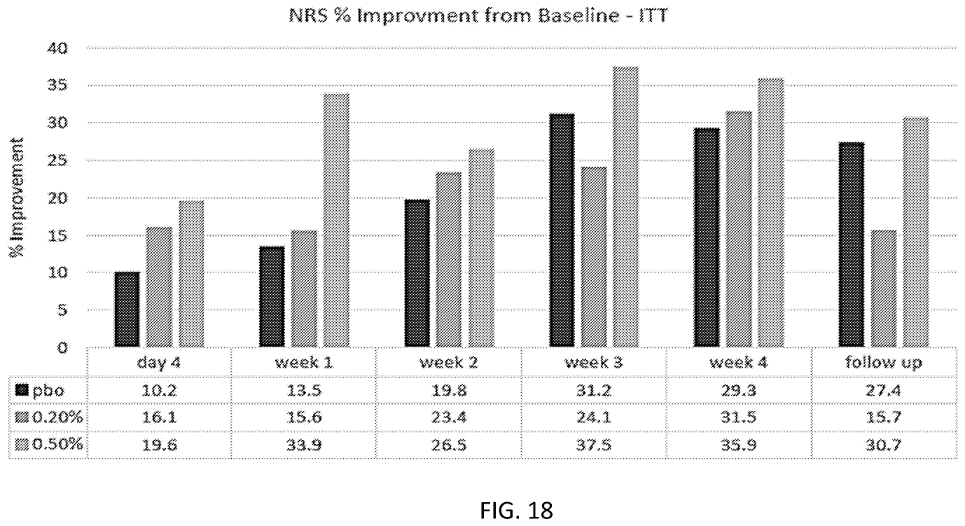

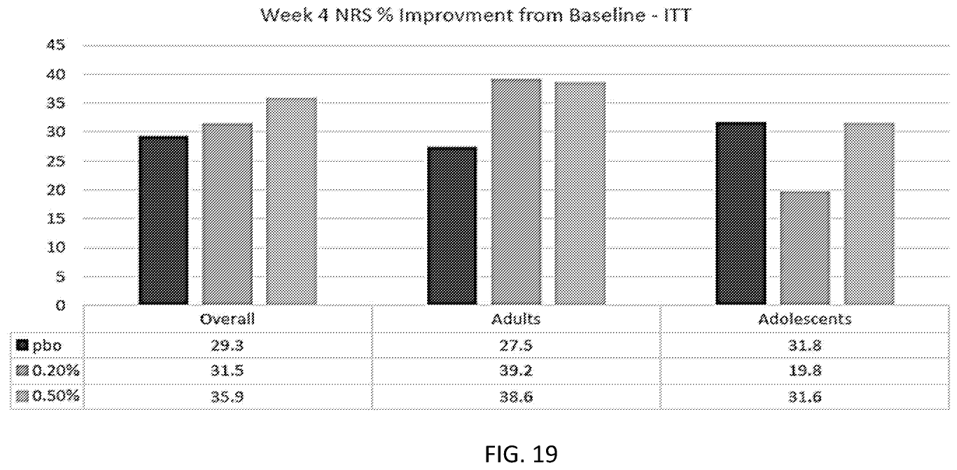

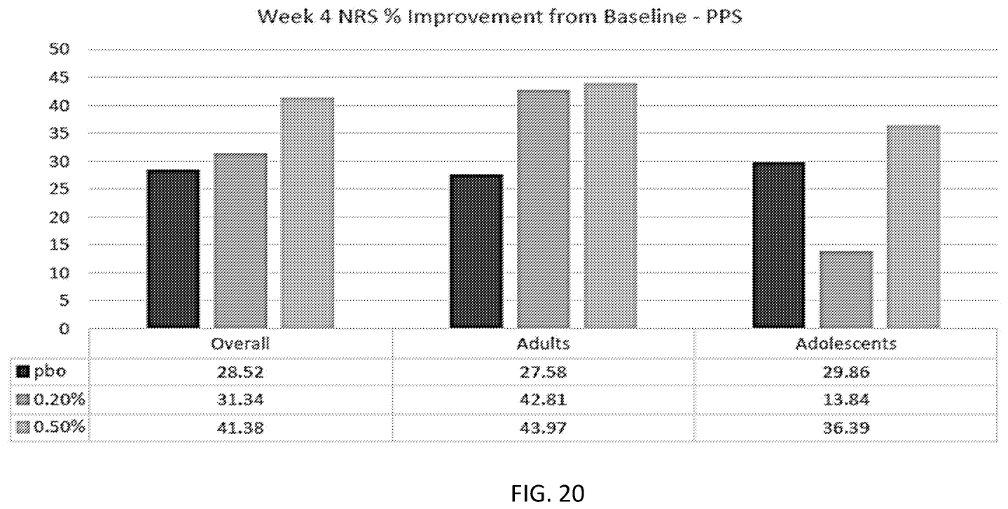

FIG. 18 shows the improvement in NRS (itch) from baseline in the ITT population. Rapid response in itch was observed by Week 1 in 0.5% group. High vehicle response was observed as early as Week 2. FIG. 19 shows the improvement in NRS (itch) from baseline at week 4 in the ITT population. FIG. 20 shows the improvement in NRS (itch) from baseline at week 4 in the PPS population. There was no clear difference among arms or age groups. However, there was a surprisingly high vehicle response rate.

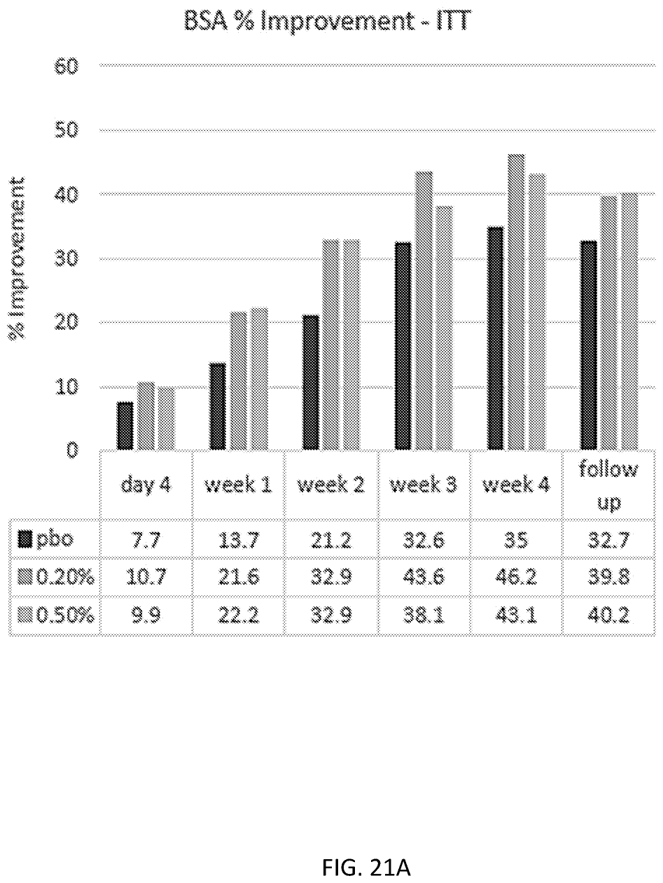

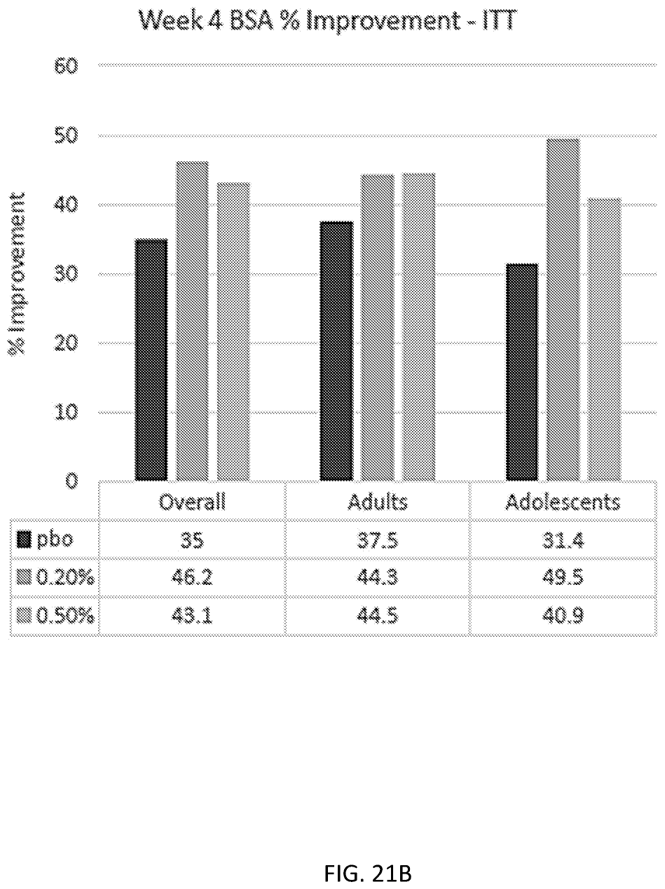

FIG. 21 shows the BSA % improvement from baseline and the week 4 BSA % improvement in the ITT population. Modest separation from vehicle observed vs. active arms across age groups at Week 4. Faster response observed in active arms.

Conclusions: RVT-501 0.2% and 0.5% ointments were well tolerated. A higher percentage of subjects in the RVT-501 treatment groups achieved an IGA score of 0.1 with at least a 2-point grade improvement (Vehicle=15.1%, 0.2% RVT-501=21.8%, 0.5% RVT-501=24.5%). Adolescent subjects treated with RVT-501 displayed greater treatment effects. No differences in treatment effect were observed in Adult subjects. Greater improvements in Itch and EASI score were observed in RVT-501 treated subjects in the first 2 weeks of treatment. This differential response between treatment groups decreased during the second 2 weeks.

In this study, both the RVT-501 0.2% and RVT-501 0.5% ointments were generally safe and well tolerated in adult and adolescent subjects with mild to moderate AD. No deaths or SAEs were reported.

Only 3 subjects had detectable levels of RVT-501 or its active M11 metabolite after 2 weeks of treatment, all near the LLQ, demonstrating minimal to no systemic absorption.

A numerically higher proportion of subjects had an improvement in IGA and achieved an IGA score of 0 or 1 with at least a 2-point improvement in both RVT-501 treatment groups as compared with the vehicle, and a dose-dependent response was observed (overall Week 4 results: 15.1% in the vehicle group, 21.8% in the RVT-501 0.2% group, and 24.5% in the RVT-501 0.5% group).

Adolescent subjects treated with RVT-501 achieved a higher IGA response than adult subjects, especially after application of RVT-501 0.5% (RVT-501 0.5% Week 4 results: 31.6% in the adolescent group, versus 20.0% in the adult group).

Numerically higher improvements in affected BSA and EASI scores were observed in the RVT-501 treated subjects when compared with the vehicle, especially for the adolescents, and in the first 2 weeks of treatment for all subjects. This differential response between treatment groups diminished during the second 2 weeks of the study due to increases in response in the vehicle group.

Improvements in pruritus were reported by the adult subjects for both tested concentrations. The improvements were less pronounced in the adolescent group.

Discussion

The main objective of this study was to evaluate the safety and pharmacokinetics of topical RVT 501 applied BID in adults and adolescents with atopic dermatitis. Efficacy of RVT-501 was also assessed as a secondary objective. A previous study conducted with a different formulation showed that RVT-501 0.2% was well tolerated and suggested that this concentration had some efficacy in the treatment AD. The current study evaluated a novel formulation at a concentration of 0.5% and included a 0.2% BID arm in the same novel formulation to control for efficacy and safety findings at the previous dose level.

A total of 157 subjects with mild to moderate atopic dermatitis were randomized (1:1:1) to one of three treatment arms, RVT-501 0.2%, RVT-501 0.5%, and vehicle, in the study (95 adults and 62 adolescents). The mean affected BSA was similar over the treatment groups at baseline (15.2% in the vehicle, 15.9% in the RVT-501 0.2% group, and 13.5% in the RVT-501 0.5% group). Most of the subjects (89.2%) had an IGA of disease severity of 3 (moderate) at baseline. The mean age, height, weight and BMI were similar across all treatment groups, and there was a higher proportion of female subjects in each group. Of note, the proportion of Black or African American subjects was slightly higher in the RVT-501 0.5% group.

RVT-501 0.2% and RVT-501 0.5% ointments were generally safe and well tolerated and no SAEs nor deaths were reported during the study. Most of the TEAEs were mild in intensity (58.1% of the reported TEAEs), 41.9% of the TEAEs were of moderate intensity, and none were severe of life-threatening. No subject experienced a TEAE of grade 3 or higher. Similar frequency and severity of TEAEs were observed between treatment groups. The majority of TEAEs were considered unrelated to study drug. A total of 14 drug-related TEAEs were reported during the study. One (1.9%) adult subject in the vehicle group (application site pain) and 1 (1.8%) adult subject in the RVT-501 0.2% (application site pruritus and application site pain) reported TEAEs that led to study discontinuation. There was a higher percentage of subjects in the adult population reporting TEAEs and drug-related TEAEs than in the adolescent population. Similarly, more TEAEs were mild in the adolescent population than in the adult population.

TEAEs that were reported by more than one subject were: nasopharyngitis (13 [8.3%] subjects), upper respiratory tract infection (8 [5.1%] subjects), application site pruritus (7 [4.5%] subjects), application site pain (5 [3.2%] subjects), nausea (3 [1.9%] subjects), dermatitis atopic nausea (3 [1.9%] subjects), headache (3 [1.9%] subjects), and vomiting (2 [1.3%] subjects). A similar number of subjects experienced application site pain and pruritus across the treatment groups. No trends were detected between treatment groups, except that the 3 subjects (1.9%) who reported dermatitis atopic (AD flare or worsening of eczema) were in the RVT-501 0.5% group. Overall, there were no trends detected between treatment groups for the safety laboratory results, vital signs, and ECGs.

Minimal or no systemic absorption was observed following topical administration of RVT-501 0.2% and 0.5% to all affected lesions. Only a small subset of subjects had measurable plasma concentrations of RVT-501 and the metabolite M11. One (1) subject had measurable RTV-501 concentrations at Week 4 and 3 subjects had measurable M11 concentrations at Week 1 or Week 4), all of which were near the LLQ, demonstrating very minimal systemic exposure.

The present study was not designed for statistically significant comparisons of the efficacy of RVT-501 versus vehicle. The main purpose of this study was to evaluate the safety and the pharmacokinetics of RVT-501 in adults and adolescents in order to gain insights on efficacy for the design of future studies in pediatric subjects.

However, results showed a higher percentage of subjects in the RVT-501 treatment groups achieved an IGA score of 0 or 1 with a 2-point improvement from baseline. Overall, endpoints involving IGA showed a numerically higher number of responders for both RVT-501 0.2% and RVT 501 0.5% compared with the vehicle. RVT-501 generally demonstrated a rapid and dose-dependent response compared to vehicle during the first 2 weeks of treatment. The differential response between treatment groups diminished starting at Week 3 due to increases in response in the vehicle group. Adolescent subjects treated with RVT-501 displayed greater treatment effects than adults.

Similar results were observed for endpoints involving BSA and EASI. These parameters showed a discrete separation between treatment arms and age groups at Week 4. BSA and EASI results also showed a rapid response from Week 0 to Week 2 in the RVT-501 treatment groups, with an increased vehicle response starting at Week 3. Adolescents displayed more pronounced results, especially when comparing RVT 501 0.2% with the vehicle. However, EASI 50 results showed a higher response for RVT 501 0.5%.

Moreover, efficacy was higher in adolescents where the proportion of IGA 0/1 with a 2-grade decrease at Week 4 was 9.1% for the vehicle, 19.0% for RVT-501 0.2%, and 31.6% for RVT-501 0.5%. This suggests that RVT-501 may be further explored as a potential treatment option for adolescents with atopic dermatitis. Further studies in younger children are needed to assess efficacy and safety in this patient population.

Results from the subject-reported pruritus NRS, the patient reported symptoms, and the POEM showed mostly positive outcomes in the adult group but not in adolescents. Pruritus NRS showed a numerical decrease in the RVT-501 0.5% group as early as Week 1 and a high vehicle response was also seen as early as Week 2. This may illustrate difficulties in using subject's self-reported outcomes in a heterogeneous adolescent population including patients as young as 11 years of age and as old as 17.

Conclusions

In this study, both the RVT-501 0.2% and RVT-501 0.5% ointments were generally safe and well tolerated in adult and adolescent subjects with mild to moderate AD. No deaths or SAEs were reported.

Only 3 subjects had detectable levels of RVT-501 or its active M11 metabolite after 2 weeks of treatment, all near the LLQ, demonstrating minimal to no systemic absorption.

A numerically higher proportion of subjects had an improvement in IGA and achieved an IGA score of 0 or 1 with at least a 2-point improvement in both RVT-501 treatment groups as compared with the vehicle, and a dose-dependent response was observed (overall Week 4 results: 15.1% in the vehicle group, 21.8% in the RVT-501 0.2% group, and 24.5% in the RVT-501 0.5% group).

Adolescent subjects treated with RVT-501 achieved a higher IGA response than adult subjects, especially after application of RVT 501 0.5% (RVT-501 0.5% Week 4 results: 31.6% in the adolescent group, versus 20.0% in the adult group).

Numerically higher improvements in affected BSA and EASI scores were observed in the RVT 501 treated subjects when compared with the vehicle, especially for the adolescents and in the first 2 weeks of treatment. This differential response between treatment groups diminished during the second 2 weeks of the study due to increases in response in the vehicle group.

Improvements in pruritus were reported by the adult subjects for both tested concentrations. The results were less clear in the adolescent group.

### Example 4: Evaluating Topical Bioavailability

Dermatopharmacokinetic (DPK) Studies

The dermatopharmacokinetic (DPK) approach is comparable to a blood, plasma, urine PK approach applied to the stratum corneum. DPK encompasses drug concentration measurements with respect to time and provides information on drug uptake, apparent steady-state levels, and drug elimination from the stratum corneum based on a stratum corneum concentration-time curve.

For antiacne drug products, target sites are the hair follicles and sebaceous glands. In this setting, the drug diffuses through the stratum corneum, epidermis, and dermis to reach the site of action. The drug may also follow follicular pathways to reach the sites of action. The extent of follicular penetration depends on the particle size of the active ingredient if it is in the form of a suspension. Under these circumstances, the DPK approach is still expected to be applicable because studies indicate a positive correlation between the stratum corneum and follicular concentrations.

Application and Removal of Test and Reference Products: The treatment areas are marked using a template without disturbing or injuring the stratum corneum/skin. The size of the treatment area will depend on multiple factors including drug strength, analytical sensitivity, the extent of drug diffusion, and exposure time. The stratum corneum is highly sensitive to certain environmental factors. To avoid bias and to remain within the limits of experimental convenience and accuracy, the treatment sites and arms should be randomized. Uptake, steady-state, and elimination phases, as described in more detail below, may be randomized between the right and left arms in a subject. Exposure time points in each phase may be randomized among various sites on each arm. The test and reference products for a particular exposure time point may be applied on sites to minimize differences. Test and reference products should be applied concurrently on the same subjects according to a SOP that has been previously developed and validated. The premarked sites are treated with predetermined amounts of the products (e.g., 5 mg/sq cm) and covered with a nonocclusive guard. Occlusion is used only if recommended in product labeling. Removal of the drug product is performed according to SOPs at the designated time points, using multiple cotton swabs or Q-tips with care to avoid stratum corneum damage. In case of certain oily preparations such as ointments, washing the area with a mild soap may be needed before skin stripping. If washing is carried out, it should be part of an SOP.

Sites and Duration of Application: The BA/BE study should include measurements of drug uptake into the stratum corneum and drug elimination from skin. Each of these elements is important to establish bioavailability and/or bioequivalence of two products, and each may be affected by the excipients present in the product. A minimum of eight sites should be employed to assess uptake/elimination from each product. The time to reach steady state in the stratum corneum should be used to determine timing of samples. For example, if the drug reaches steady-state in three hours, 0.25, 0.5, 1 and 3 hours posttreatment may be selected to determine uptake and 4, 6, 8 and 24 hours may be used to assess elimination. A zero time point (control site away from test sites) on each subject should be selected to provide baseline data. If the test/reference drug products are studied on both forearms, randomly selected sites on one arm may be designated to measure drug uptake/steady-state. Sites on the contralateral arm may then be designated to measure drug elimination. During drug uptake, both the excess drug removal and stratum corneum stripping times are the same so that the stratum corneum stripping immediately follows the removal of the excess drug. In the elimination phase, the excess drug is removed from the sites at the steady-state time point, and the stratum corneum is harvested at succeeding times over 24 hours to provide an estimate of an elimination phase.

Collection of Sample: Skin stripping proceeds first with the removal of the first 1-2 layers of stratum corneum with two adhesive tapes strip/disc applications, using a commercially available product (e.g., D-Squame, Transpore). These first two tape-strip(s) contain the generally unabsorbed, as opposed to penetrated or absorbed, drug and therefore should be analyzed separately from the rest of the tape-strips. The remaining stratum corneum layers from each site are stripped at the designated time intervals. This is achieved by stripping the site with an additional 10 adhesive tape-strips. All ten tape strips obtained from a given time point are combined and extracted, with drug content determined using a validated analytical method. The values are generally expressed as amounts/area (e.g., ng/cm²) to maintain uniformity in reported values. Data may be computed to obtain full drug concentration-time profiles, Cₘₐₓ₋ₛₛ, Tₘₐₓ₋ₛₛ, and AUCs for the test and reference products.

Procedure for Skin Stripping:

To assess drug uptake: Apply the test and/or reference drug products concurrently at multiple sites. After an appropriate interval, remove the excess drug from a specific site by wiping three times lightly with a tissue or cotton swab. Using information from the pilot study, determine the appropriate times of sample collection to assess drug uptake. Repeat the application of adhesive tape two times, using uniform pressure, discarding these first two tape strips. Continue stripping at the same site to collect ten more stratum corneum samples. Care should be taken to avoid contamination with other sites. Repeat the procedure for each site at other designated time points. Extract the drug from the combined ten skin strippings and determine the concentration using a validated analytical method. Express the results as amount of drug per square cm treatment area of the adhesive tape.

To assess drug elimination: Apply the test and reference drug product concurrently at multiple sites chosen based on the results of the pilot study. Allow sufficient exposure period to reach apparent steady-state level. Remove any excess drug from the skin surface as described previously, including the first two skin strippings. Collect skin stripping samples using ten successive tape strips at time intervals based on the pilot study and analyze them for drug content.

Metrics and Statistical Analyses: A plot of stratum corneum drug concentration versus a time profile should be constructed to yield stratum corneum metrics of Cₘₐₓ, Tₘₐₓ and AUC. The two one-sided hypotheses at the α=0.05 level of significance should be tested for AUC and Cₘₐₓ by constructing the 90 percent confidence interval (CI) for the ratio between the test and reference averages. Individual subject parameters, as well as summary statistics (average, standard deviation, coefficient of variation, 90% CI) should be reported. For the test product to be BE, the 90 percent CI for the ratio of means (population geometric means based on log-transformed data) of test and reference treatments should fall within 80-125 percent for AUC and 70-143 percent for Cₘₐₓ.

In Vivo Dermal Open Flow Microperfusion

In dermal open-flow microperfusion (dOFM), a thin, hollow tube is inserted just under the skin surface, running through a section of the skin a few inches wide and then exiting. A liquid similar to body fluid is injected into the tubing; a portion of the tube under the skin is porous, so any drug that has been applied and absorbed through the skin's outer layer enters the flowing liquid, which is then collected for analysis. dOFM can reliably measure the changing amounts of drug in the skin after topical application of a dermatological drug product.

### Example 5: A Dried Blood Spot Assay with UPLC-MS/MS for the Simultaneous Determination of E6005, a Phosphodiesterase 4 Inhibitor, and its Metabolite in Human Blood

E6005, a novel phosphodiesterase 4 inhibitor, is currently under clinical development for the treatment of atopic dermatitis. To support pediatric clinical trials, dried blood spots assay for the simultaneous determination of E6005 and its main metabolite, ER-392710 (M11), has been developed using ultra-performance liquid chromatography with tandem mass spectrometry. E6005 and M11 in 25 μL blood spotted onto FTA™ DMPK-C cards were extracted by simple protein precipitation with water/acetonitrile (1:1, v/v), and then chromatographed on a reversed phase column under gradient elution. The mass transitions m/z 473.1→163.0 for E6005 and m/z 459.1→149.0 for M11 were monitored in a positive ion electrospray ionization mode. E6005 and M11 were quantifiable from 1 to 200 ng/mL on dried blood spots. Accuracy and precision in the intra- and inter-batch reproducibility were within the acceptance criteria recommended by the bioanalytical guidelines. Impacts on the assay accuracy by hematocrit and blood spot volume were evaluated and results showed hematocrit impacted the analytes' accuracy. Various stability assessments including possible conversion of E6005 to M11 were thoroughly performed. The method was successfully applied to determine E6005 and M11 levels in blood samples in support of a pediatric clinical trial.

Phosphodiesterase 4 (PDE4) is expressed on various inflammatory cells and considered to play a critical role in the inflammatory disorders including atopic dermatitis. E6005 potently inhibited human PDE4 with an IC50 of 2.8 nM and also demonstrated efficacy in mice and humans, thus E6005 is considered as a promising drug for the treatment of atopic dermatitis. Atopic dermatitis is one of autoimmune diseases and a number of children and infants are suffering from. Although it is important to monitor drug concentrations in children and infants, the volume of blood sampling is limited. Dried blood spots (DBS) is a technique of micro-samplings in which small aliquots of whole blood samples were spotted onto filter paper for analysis of drugs' levels and has been applicable for the analysis of a wide spectrum of drugs. DBS has many advantages over conventional plasma assay. It is less laborious in sample preparation of DBS compared to plasma based assay in which blood samples collected were centrifuged to obtain plasma samples for the assay. In addition, DBS requires smaller volume of blood (less than 100 μL) than conventional blood sampling (typically more than 1 mL) in typical plasma based assays.

In vitro metabolism studies demonstrated that E6005 was metabolized to various metabolites including M11 and clinical studies showed that systemic exposure of M11 in plasma was comparable to or more than that of E6005. Therefore, in a human DBS assay as well, a simultaneous assay method for the determination of E6005 and M11 has been developed and validated.

### Materials and Methods

Materials: E6005 and M11 were synthesized at Eisai Co., Ltd. (Ibaraki, Japan). ER-497652 and ER-497653 used as an internal standard (IS) for E6005 and M11, respectively, were synthesized at Sekisui Medical Co., Ltd. (Ibaraki, Japan). Blank human whole blood with EDTA-2K as an anticoagulant was obtained from volunteers in Eisai Co., Ltd. with written consent. Blank human plasma was prepared by centrifuging aliquots of whole blood obtained or commercially available one was purchased from Biopredic International (Saint Grégoire, France). High-performance liquid chromatography (HPLC) grade acetonitrile, methanol, distilled water, and ammonium formate as well as a special grade formic acid were purchased from Wako Pure Chemical Industries, Ltd. (Osaka, Japan). All other chemicals used were of analytical grade. FTAE DMPK-C blood spots cards and equipment used for disc punching including a punching device, Harris Micro Punch 3.0 mm, and cutting mat, Harris cutting mat, were purchased from GE Healthcare (Buckinghamshire, UK). Silica-gel desiccant and polyethylene bag for storing DBS cards were purchased from Toyotakako Co., Ltd. (Aichi, Japan) and Asahi Kasei home products Co. (Tokyo, Japan), respectively.

Assay conditions: The analytical conditions of E6005 and M11 in DBS were the same as those used for the validated assay in plasma. Briefly, an Acquity system (Waters, Mass., USA) coupled with triple quadrupole mass spectrometer Quattro Premier (Waters) was used as an ultra-performance liquid chromatography (UPLC) with tandem mass spectrometry (ULPC-MS/MS). E6005, M11, and IS were eluted with the mobile phase consisting of (A) water/acetonitrile/1 mol/L ammonium formate (950:50:5, v/v/v) and (B) water/acetonitrile/1 mol/L ammonium formate (100:900:5, v/v/v) and chromatographed on an Acquity UPLC BEH C18 column (2.1 mm×100 mm, 1.7 μm, Waters) maintained at 40° C. The gradient program is as follows: a linear increase of mobile phase (B) from 5% to 95% for 4.0 min, then an isocratic elution of 95% (B) for 0.5 min, followed by having the system equilibrated with 5% (B) for 1.5 min. The flow rate was 0.25 mL/min to 4.5 min then increased to 0.3 mL/min for equilibrium.

The optimized mass spectrometer conditions in the multiple reaction monitoring were 370° C. for desolvation temperature, 125° C. for source temperature, and 1.3 kV for capillary voltage, 65 V for cone voltage, and −55 eV for collision energy. The mass transition m/z (precursor ion→product ion) 473.1→163.0, m/z 459.1→149.0, m/z 477.2→167.0, and m/z 463.2→153.0 were monitored for E6005, M11, IS of E6005, and IS of M11, respectively.

Preparation of calibration and quality control samples: A mixture of stock solutions of E6005 and M11 in methanol (each 100 μg/mL as free base) was diluted with acetonitrile/methanol (1:1, v/v) to prepare working standard solutions. By fortifying working solutions to blank naïve whole blood (hematocrit: ca. 45%), calibration samples were prepared at concentrations of 1, 2, 10, 20, 80, 100, 160, and 200 ng/mL for both E6005 and M11. Fresh blank whole blood was used to prepare calibration samples otherwise stated. A working solution of the IS in acetonitrile/methanol (1:1, v/v) was prepared in the similar way as described above (200 ng/mL). The working solutions were stored below −20° C. and used within 181 days in which stability was confirmed. Quality control (QC) samples including the lower limit of quantification (LLOQ), low QC (LQC), middle QC (MQC), and high QC (HQC), were prepared at concentrations of 1, 3, 30, and 160 ng/mL blood with designated hematocrit values. Blood samples with varying hematocrits were prepared by mixing plasma and blood cells with the nominal ratios of 80:20 to 30:70 (v/v). Accurate hematocrit values determined using a hematology analyzer (ADVIA 120, Siemens, Munich, Germany) were 19.3, 26.9, 36.2, 46.7, 49.1, 51.8, 57.5, and 63.6% for the nominal hematocrit of 20% (80:20), 30% (70:30), 40% (60:40), 50% (50:50), 53% (47:53), 56% (44:56), 60% (40:60), and 70% (30:70), (plasma/blood cells, v/v), respectively. Aliquots (25 μL) of blood samples (calibration samples and QC samples) were spotted onto the center of circle of FTA™ DMPK-C cards using a calibrated pipette to prepare DBS. The cards were allowed to dry at room temperature for at least 2 h. QC samples used for the long-term stability assessment were stored at designated temperature in a sealed polyethylene bag containing Silica-gel desiccant.

Sample extraction procedures: DBS discs (i.d. 3 mm) were punched out at the center of spots using the punching device, Harris Micro Punch, into tubes for extraction. A 10 μL aliquot of the IS working solution (200 ng/mL) was spiked and then the analytes were extracted by 100 μL acetonitrile/water (1:1, v/v). After vigorously vortexing, samples were centrifuged (15700×g, 1 min) at 4° C. to obtain supernatants for injection. A 10 μL aliquot of supernatants was injected to the UPLC-MS/MS system.

### Method Validation

Linearity: Punched discs spotted with calibration samples (1-200 ng/mL for both E6005 and M11) were extracted and assayed to determine inaccuracy (relative error, RE) at each concentration across 8 assay runs. Inaccuracy of determined E6005 and M11 at each concentration should be within ±15% (±20% was allowed for the LLOQ). Imprecision as relative standard deviation (RSD) was also calculated and checked whether % RSD was 15% or less (20% or less was allowed for the LLOQ).

Specificity: Discs spotted with blank human blood from six individuals were extracted to check if there were any endogenous peaks interfering with assay of the analytes. Interfering peak areas should be less than 20% for E6005 and M11 while 5% for IS of those of LLOQ samples.

Intra- and inter-batch reproducibility: Inaccuracy and imprecision of E6005 and M11 were determined using QC samples (LLOQ, LQC, MQC, and HQC) in the intra- and inter-assay batch. Five replicates per concentration were assessed for the intra-batch reproducibility, and intra-batch evaluation was repeated across three batches for the inter-batch reproducibility. The acceptance criteria for inaccuracy and imprecision were within ±15% and 15%, respectively (±20% for inaccuracy and 20% for imprecision are allowed for the LLOQ samples).

Extraction recovery and matrix effect: Extraction recovery of E6005 and M11 from DBS discs was assessed at three concentrations (3, 30, and 160 ng/mL, three replicate/concentration), while recovery of the IS from the system was determined at 60 ng/mL. Extraction recovery of the analytes was determined by dividing the peak area of the analytes spiked to blank blood prior to extraction by that spiked after extraction (reference samples) taking the differences in areas between discs for extraction and blood spots into account while extraction recovery of the IS was determined just by comparison of peak area between the extracted samples and reference ones without any correction. Blood spot areas were calculated by πr², where r is radius of spots determined by a ruler.

Matrix factors were evaluated by dividing peak area of reference samples from six individuals by that of neat solution with identical concentrations. Matrix factors were determined for the analytes of interest (E6005 and M11) at 3 ng/mL and the corresponding IS at 160 ng/mL. IS-corrected matrix factors of E6005 and M11 were calculated by dividing matrix factor of E6005 and M11 by that of the corresponding IS. The % RSD of the IS-corrected matrix factor should be within 15%.

Effect of blood spot volume, hematocrit, and punching location: As potential impacts on assay accuracy by blood spot volume, hematocrit, and punching location are unique to DBS-associated bioanalytical method validation studies, these parameters were also assessed. To assess potential impacts by blood spot volume, various volume of QC samples (10, 20, 25, 30, and 40 μL) at low (3 ng/mL) and high (160 ng/mL) concentrations were spotted onto the circle of DBS cards, and then center-punched discs were assayed in three replicates against the calibration samples with the fixed volume (25 μL). Acceptable blood spot volumes should have inaccuracy ≤±15%.

Effects of hematocrit on the assay of E6005 and M11 were evaluated using blood samples with varying hematocrit values (19.3% to 63.6%) at low (3 ng/mL) and high (160 ng/mL) concentrations with the other conditions fixed (25 μL spot volume and center-punching). DBS discs spiked by blood with varying hematocrit were assayed in three replicates against calibration samples prepared from naïve blood (hematocrit: 45.1%). Potential relationship between spot area and inaccuracy of QC samples was evaluated. Impacts of hematocrit were considered negligible when the inaccuracy was not greater than ±15%.

Potential impacts of punching locations in discs were assessed for the following four locations of the spot with the other condition fixed (25 μL spot volumes and 45.1% hematocrit): upper right, lower right, upper left, and lower left. Low (3 ng/mL) and high (160 ng/mL) concentrations were evaluated. Discs punched out from four locations were assayed with the center-punched disc of calibration samples. No impact of punching location was suggested when the inaccuracy was within ±15%.

Carryover: Two types of carryover assessments should be evaluated in bioanalytical methods using DBS-based assays; one is carryover derived from repetitive sample injection via UPLC, a typical validation parameter in the method validation, and the other is DBS-specific spot-to-spot carryover mainly derived from punching devices by repetitive punching of discs. The carryover in the UPLC was assessed by injecting blank samples just after upper limit of quantification (ULOQ) samples. The other possible carryover caused by repetitive punching was investigated by punching discs with blank samples just after the ULOQ samples using a punching device without any wash. Peak areas of any interferences in blank samples should be less than 20% and 5% of the LLOQ sample for the analytes of interest and the IS, respectively.

Stability: Following stability of E6005 and M11 in DBS was assessed at low and high concentrations using LQC and HQC samples (three replicates/concentration): bench-top stability for 7 days at room temperature, long-term frozen stability for 160 days at room temperature and below −15° C., and processed sample stability for 85 h at 4° C. To investigate impacts of high humidity on the stability, bench-top stability test was performed at room temperature with relative humidity of ca. 80%-84%. Samples were considered stable when % bias from the nominal concentrations was within ±15%.

As a part of the stability assessment, possible conversion of E6005 to M11 was also investigated using HQC samples in which only E6005 was fortified. After designated times, formed M11 concentrations were determined and percentage of conversion was calculated by dividing M11 concentrations by E6005 concentrations on the molar concentration basis.

Shelf-life of refrigerated blood: As it is sometimes a challenge that fresh blood samples are available to prepare calibration or QC samples, it is of interest to know whether or not refrigerated blood can be used. The shelf-life of refrigerated blood was assessed by assaying QC samples at low (3 ng/mL) and high (160 ng/mL) concentrations in three replicates prepared from refrigerated blood for seven days against calibration samples prepared from fresh blood. The refrigerated blood can be used when % bias from the nominal concentrations was within ±15%.

Clinical application: A clinical study was performed in which E6005 ointment containing 0.05% or 0.2% was topically applied twice a day for two weeks to pediatric subjects. Blood samples were obtained at 1- and 2-week post-dose as well as subsequent 7-day follow-up period in collection tubes with K2-EDTA as an anticoagulant, thereafter put on ice as soon as possible to reduce possible conversion of E6005 to M11. Details on sample handling at the clinics were clarified in a lab manual; a 25 μL aliquot blood sample was spotted onto the center of circle of DBS cards (four replicates per sample) at clinics, then dried at room temperature for at least 2 h. DBS cards with desiccants were placed in zip lock bags and stored frozen below −20° C. until shipment to a bioanalytical laboratory. Samples were stored below −15° C. at the laboratory until they were subjected to sample processing for the determination of E6005 and M11 concentrations in DBS.

### Results and Discussion

Method Development

Blood spotting is one of the crucial steps in the DBS method to ensure accurate determination, thus in the method development, some abuses on blood spotting were investigated. Typically blood samples with drugs of interest were spotted by one drop per spot with a pipette. As an abuse of double drop may be possible at clinics, DBS with the double drop of blood samples (each 15 μL aliquots) was processed and concentrations of E6005 and M11 were determined against calibration samples with single blood drop (30 μL aliquots) to ensure whether the RE (%) was within 15%. The RE (%) of double drop samples was −4.6% and 3.7% for E6005 and M11, respectively, suggesting minimal impacts of double drop of blood samples as long as the total volume is comparable. The laboratory manual indicates that pipettes should be kept just above the DBS paper not touched when spotting, however blood spot may be performed with pipettes touched on cards; the % RE of E6005 and M11 was 4.3% and 9.7%, respectively, indicating minimal impacts by pipette’ touching on card when spotting.

Extraction procedure focused on selecting appropriate extraction solvents: acetonitrile, acetonitrile/water (8:2, v/v), acetonitrile/water (1:1, v/v), methanol, methanol/water (8:2, v/v), and methanol/water (1:1, v/v). Although minimal extraction was noted with acetonitrile, other solvents showed similar extraction efficiency. Less endogenous peaks in chromatograms led to the selection of 50% acetonitrile rather than pure organic solvents or higher organic solvent containing solvents.

One possible issue to be addressed in the method development is lower sensitivity in DBS method due to lower volume of matrix compared to traditional plasma assay. Given the target LLOQ (1 ng/mL blood), increases in punching spot area were tested for whether higher sensitivity could be achieved. Other than 3 mm diameter disc punching, 6 mm diameter punching was assessed and peak intensity of the analytes was increased 3 to 4 folds which was comparable to the theoretical increase (4 folds).

Method Validation

Linearity and selectivity: E6005 and M11 were quantifiable ranging from 1 to 200 ng/mL with acceptable % inaccuracy and imprecision at all the concentrations tested (Table 18). Calibration curves were consistent among assay batches with minimal variability of the slope (8.2% and 8.6% for E6005 and M11, respectively).

TABLE 18
Linearity of E6005 and M11 in human dried blood spots

| | E6005 | | M11 | |
|---|---|---|---|---|
| Concentration | Inaccuracy | Imprecision (% | Inaccuracy | Imprecision (% |
| (ng/mL) | (% RE) | RSD) | (% RE) | RSD) |
| 1 | −0.3 | 1.4 | 2.6 | 3.3 |
| 2 | 0.9 | 2.9 | −4.8 | 7.0 |
| 10 | −1.2 | 3.9 | −1.8 | 3.7 |
| 20 | −0.5 | 2.7 | −0.8 | 2.3 |
| 80 | −0.1 | 4.4 | 0.4 | 4.0 |
| 100 | −0.8 | 3.0 | 0.2 | 2.2 |
| 160 | 0.5 | 5.1 | 1.4 | 2.6 |
| 200 | 1.3 | 5.0 | 2.8 | 4.6 |

Inaccuracy and imprecision as the relative standard deviation (RSD) were calculated from 8 analytical runs.

Accuracy and precision: Intra- and inter-batch accuracy and precision were assessed at four levels (LLOQ, LQC, MQC, and HQC) and results are shown in Table 19. The inaccuracy as % RE and imprecision as % RSD for E6005 and M11 were within ±7.0% and 9.6% in the intra-batch test and within ±8.0% and 15.7% (at the LLOQ) in the inter-batch test, respectively. These results were within the acceptance criteria recommended by the bioanalytical guidelines from US Food and Drug Administration and European Medicines Agency. No dilution integrity was assessed since it was highly unlikely that the analytes' levels exceeded the ULOQ (200 ng/mL) in clinical studies.

TABLE 19
Intra- and inter-batch reproducibility of
E6005 and M11 in human dried blood spots

| | Nominal | | | | |
|---|---|---|---|---|---|
| Quality | concentration | Inaccuracy (%) | | Imprecision (%) | |
| control | (ng/mL) | E6005 | M11 | E6005 | M11 |

Intra-batch (n = 5/batch)

| | | | | | |
|---|---|---|---|---|---|
| LLOQ | 1 | 4.0 | 7.0 | 9.6 | 4.7 |
| LQC | 3 | −1.3 | 1.0 | 4.4 | 5.9 |
| MQC | 30 | 6.3 | 5.0 | 6.3 | 3.2 |
| HQC | 160 | −1.3 | −1.3 | 6.3 | 4.4 |

Inter-batch (n = 15, n = 5/batch, 3 batches in total)

| | | | | | |
|---|---|---|---|---|---|
| LLOQ | 1 | 7.0 | 8.0 | 14.0 | 15.7 |
| LQC | 3 | 0.7 | 1.0 | 7.3 | 7.6 |
| MQC | 30 | 1.3 | 0.3 | 6.3 | 6.0 |
| HQC | 160 | 1.9 | 1.9 | 5.5 | 5.5 |

Extraction recovery and matrix effect: Table 20 shows extraction recoveries of the analytes of interest and the IS. Extraction recoveries of E6005 and M11 at low, middle, and high concentrations were 79.2%-86.7% and 73.3%-87.5%, respectively, by taking the differences in disc areas between extracted and spotted into account. The recovery of the IS was 93.7% for E6005 and 96.9% for M11. Extraction recoveries of E6005 and M11 were consistent across the concentrations tested. Relatively lower extraction of the analytes than the IS was attributable to differences in fortifying neat solution in the system, where the analytes were spotted onto cards before extraction while the IS was just fortified after extraction of the analytes.

TABLE 20
Mean extraction recovery of E6005 and
M11 and the internal standards (IS)

| Nominal | |
|---|---|
| concentration | % Mean recovery |

| Samples | (ng/mL) | E6005 | M11 |
|---|---|---|---|
| LQC | 3 | 81.5 ± 1.5 | 80.9 ± 4.4 |
| MQC | 30 | 79.2 ± 2.7 | 73.3 ± 2.3 |
| HQC | 160 | 86.7 ± 6.5 | 87.5 ± 7.5 |
| IS | 60 | 93.7 ± 4.9 | 96.9 ± 4.5 |

Data represent the mean ± standard deviation of three replicates for the analytes at each level and nine replicates for the IS.

Matrix effects of the analytes and the IS were evaluated using blood from six individuals. Neither ion suppression nor ion enhancement were observed for both analytes and the IS with matrix factor ranging from 92.5% to 100.3%. The IS-normalized matrix factor was almost unity ranged from 93.2% to 99.2% with CV of 2.2% for E6005 and 2.1% for M11, indicating no matrix effects (Table 21).

TABLE 21
Matrix effects of E6005 and M11 in human
dried blood spots from six individuals

| | |
|---|---|
| Matrix factor | IS-normalized matrix factor |

| Lot | E6005 | M11 | E6005 | M11 |
|---|---|---|---|---|
| 1 | 0.925 | 0.966 | 0.992 | 0.983 |
| 2 | 0.971 | 0.951 | 0.975 | 0.956 |
| 3 | 0.927 | 0.968 | 0.957 | 0.970 |
| 4 | 0.951 | 0.935 | 0.998 | 0.932 |
| 5 | 0.909 | 0.928 | 0.945 | 0.936 |
| 6 | 0.940 | 0.933 | 0.986 | 0.960 |
| CV(%) | 2.3 | 1.8 | 2.2 | 2.1 |

Matrix effects were evaluated at 5 ng/mL for E6005 and M11.

Impacts of blood spot volume, hematocrit, and punching location: Possible impacts of blood spot volume were investigated by spotting blood samples (10 to 40 μL) containing E6005 and M11 at low and high concentrations. The % RE of both E6005 and M11 was within the acceptance criteria (<±15%) when 10 to 30 μL blood was spotted. On the other hand, inaccuracy of M11 at 40 μL spotting was slightly higher than the criteria (16.3%). These results suggest that blood spot volume was ensured at least up to 30 μL.

Impacts of hematocrit were also assessed using blood samples with varying hematocrit (19.3%-63.6%). The % RE of E6005 and M11 was within <±15% at hematocrit ranging from 26.9% to 51.8%. On the other hand, inaccuracy negatively biased at the hematocrit 19.3% while positively biased at the hematocrit 57.5% and 63.6%. It would be explained by varying viscosity of blood; the diffusion of blood on DBS cards would be higher for blood with smaller hematocrit, thus resulted in larger blood spot area. As the same size of discs were punched out regardless of blood spot area, larger blood spot area leads to lower concentrations of analytes, and vice versa. The impact of hematocrit on the assay accuracy of E6005 and M11 was not clinically significant given that hematocrit of subjects determined in a clinical study ranged from 0.33 to 0.47.

As punching locations of a disc in a spot may impact the assay of E6005 and M11, the % RE of the analytes in four different punching locations (upper, lower, right, and left of spots) was compared to that in the center of spots. Discs punched out from all the peripheral location showed % RE within 15% when concentrations were determined against calibration samples punched from the center of disc, indicating minimal impact of disc punching locations.

Carryover: No carryover derived from repetitive sample injection was detected in chromatograms of blank samples injected just after ULOQ samples. No DBS-specific device-oriented carryover was also noted.

Stability: Results of the stability assessment of E6005 and M11 in DBS are shown in Table 22. A bench-top stability test demonstrated that E6005 and M11 were stable for 160 days at ambient temperature. Long-term frozen stability was assessed below −15° C. and confirmed stable up to 160 days. Stability of E6005 and M11 in processed samples was confirmed for 85 h when stored at 4° C. No impacts of high humidity on the stability were also ensured for 7 days at ambient temperature with relative humidity of ca. 80%-84%. Possible conversion of E6005 to M11 was evaluated by fortifying only E6005 in blood and formed M11 levels were assessed (Table 23). The conversion of E6005 in the long-term stability test was slightly higher at room temperature (2.2%) compared to that stored below −15° C. (1.2%). However, the conversion was not so different between 7 and 160 days even when stored at room temperature (1.0% and 2.2% for 7 and 160 days, respectively). The minimal conversion of E6005 to M11 was not clinically significant given minimal exposure of E6005 after dermal application (1.65 ng/mL at the maximum).

TABLE 22
Stability of E6005 and M11 in human dried blood spots

| | | Quality control | | % bias | |
|---|---|---|---|---|---|
| Stability test | Condition | samples | | E6005 | M11 |
| Bench-top | After 7 days at | LQC | (3 ng/mL) | 4.0 | 6.0 |
| | RT (room | HQC | (160 ng/mL) | −1.3 | 2.5 |
| | temperature) | | | | |
| Under high | After 7 days at | LQC | (3 ng/mL) | −2.0 | −2.3 |
| humidity | humidity 80-84% | HQC | (160 ng/mL) | −9.4 | −12.5 |
| Long-term | After 160 days | LQC | (3 ng/mL) | −1.7 | −1.3 |
| | below −15° C. | HQC | (160 ng/mL) | −3.1 | −1.3 |
| Long-term | After 160 days at | LQC | (3 ng/mL) | −0.7 | 0.3 |
| | RT | HQC | (160 ng/mL) | −3.1 | 9.4 |
| In processed | After 85 h | LQC | (1 ng/mL) | 6.8 | 5.9 |
| samples | at 4° C. | HQC | (160 ng/mL) | 1.2 | 0.2 |

Quality control samples at low (3 ng/mL) and high (160 ng/mL) levels were assayed in triplicates and the relative error was calculated from the mean. Percent bias was calculated against nominal concentrations.

TABLE 23
Conversion of E6005 to M11 in human dried blood spots

| | | Concentration | Conversion |
|---|---|---|---|
| Stability test | Condition | (ng/mL) | (%) |
| Bench-top in blood | 7 days at RT (room | 160 | 1.0 |
| | temperature) | | |
| | 33 days at RT | 160 | 1.2 |
| | 91 days at RT | 160 | 2.1 |
| | 160 days at RT | 160 | 2.2 |
| Frozen in blood | 33 days | 160 | 1.0 |
| | below −15° C. | | |
| | 91 days | 160 | 1.2 |
| | below −15° C. | | |
| | 160 days | 160 | 1.2 |
| | below −15° C. | | |
| In processed | 85 h at 4° C. | 160 | 0 |
| samples | | | |

Blood-to-plasma partition and shelf-life of refrigerated blood: The blood-to-plasma partition (B/P) of E6005 and M11 was determined by assaying concentrations of E6005 and M11 in whole blood samples and plasma samples prepared from blood samples by centrifugation. The B/P of E6005 was 0.690 and 0.669 at 3 and 160 ng/mL, respectively, while that of M11 was 0.594 and 0.574 at 3 and 160 ng/mL, respectively, suggesting that no concentration-dependent B/P was observed. The average B/P of two levels was 0.679 for E6005 and 0.584 for M11.

The shelf-life of refrigerated whole blood was assessed by evaluating the inaccuracy of QC samples from “aged blood” fortified with E6005 and M11 at low and high levels. The % RE of E6005 was 7.7% and −1.9% at 3 and 160 ng/mL, respectively, and that of M11 was 7.3% and −3.8% at the low and high concentrations, respectively. These results suggest that aged blood stored refrigerated for seven days can be used in preparing calibration samples and QC samples.

Clinical application: Concentrations of E6005 and M11 in blood were determined in support of a pediatric clinical trial in which E6005 was topically applied. A total of 147 DBS samples were assayed according to the method described above and all the samples except one were below the LLQ. The maximum level was 1.65 ng/mL for E6005 while that of M11 was below the LLOQ. These results demonstrated that the systemic exposures of E6005 and M11 were minimal when E6005 was applied topically to children, which was similar to findings in adults. The % RE of all the calibration samples and QC samples assayed with post-dose samples was within 15%, indicating accurate determination of E6005 and M11 in sample assay.

### Conclusions

Results in the validation study demonstrated that the developed DBS method with UPLC-MS/MS for the simultaneous determination of E6005 and M11 in human whole blood is simple, selective, and reproducible. The validated method has been successfully applied to clinical studies in which blood E6005 levels in pediatrics were accurately determined using only 25 μL blood.

### Example 6: Phase 2 Study to Evaluate the Efficacy, Safety, and Tolerability of RVT-501 Topical Ointment in Pediatric Patients with Mild to Moderate Atopic Dermatitis

Study Design: Multicenter, randomized, vehicle-controlled, double-blind efficacy, safety, and tolerability study. The study consisted of four phases: Screening (up to 30 days), Double-Blind Phase (approximately 28 days), Open-Label Extension Phase (approximately 28 days), and Follow-up (5-9 days).

Objectives: Primary: To assess the efficacy of topical RVT-501 in pediatric subjects with mild to moderate atopic dermatitis. Secondary: To evaluate the safety of topical RVT-501 in pediatric subjects with mild to moderate atopic dermatitis, To assess the pharmacokinetics (PK) of topical RVT-501 in 2 to 11 years old subjects with atopic dermatitis.

Study Design/Methodology: This was a multicenter, randomized, vehicle-controlled, double-blind, Phase 2 study to evaluate the efficacy and safety of RVT-501 in pediatric subjects with mild to moderate atopic dermatitis.

There was a Double-Blind Phase lasting 4 weeks in which subjects received either RVT-501 0.5% ointment or vehicle ointment (study medication). All subjects who completed the Double-Blind Phase were eligible to enter an Open-Label Extension Phase and received the active treatment (RVT-501 0.5% ointment) for 4 weeks during the extension.

All subjects underwent screening procedures within 30 days prior to randomization to confirm eligibility. At Day 0 (baseline), eligible subjects were to be randomized (1:1) to one of two treatment arms.

During the Double-Blind Phase, subjects/caregivers applied study medication to affected areas twice daily for 4 weeks. Subjects returned to the clinic at Weeks 1, 2, and 4 for safety and efficacy assessments. A phone call was conducted at Week 3 to assess subject safety, concomitant medications, and continued participation in the trial.

Subjects/caregivers liberally applied sufficient study medication to completely cover each lesion with a thin layer of medication. Medication was applied to all affected areas, including newly appearing lesions and lesions that improved during the study.

Subjects who completed the Double-Blind Phase could elect to enroll in the optional Open-Label Extension Phase upon completing the Week 4 visit assessments.

Subjects/caregivers who chose to participate were dispensed RVT-501 0.5% ointment at the Week 4 visit and continued to apply the ointment to all treatment areas twice daily for 4 weeks during the extension.

Subjects/caregivers returned to the clinic at Weeks 6 and 8 for safety and efficacy assessments. A phone call was conducted at Week 5 to assess subject safety, concomitant medications, and continued participation in the study.

There was a follow-up assessment 5 to 9 days following the completion of the Double-Blind Phase for subjects who chose not to enroll in the Open-Label Extension Phase, or following completion of the Open-Label Extension Phase, as applicable.

Target Population: Approximately 100 pediatric subjects with atopic dermatitis aged 2 to 17 years were to be enrolled in this study.

Main Criteria for inclusion: Male and female pediatric subjects aged 2 to 17 with confirmed diagnosis of atopic dermatitis by Hanifin and Rajka criteria. Subjects with atopic dermatitis covering 5% to 40% of the Body Surface Area (BSA) and with an Investigator Global Assessment (IGA) of disease severity of 2 or 3 (mild or moderate atopic dermatitis) at baseline. History of atopic dermatitis and stable disease for at least 1 month according to the subject or caregiver.

Compound: RVT-501 0.5% ointment, applied twice daily for 28 days plus an additional 28 days for subjects who entered the Open-Label Extension Phase, Formulation C2 (see Table 1). Vehicle ointment, applied twice daily for 28 days, Formulation B (see Table 1).

Criteria for Evaluation/Endpoints

Primary Efficacy Endpoint: Proportion of subjects who achieved an IGA of 0 or 1 and at least a 2-point improvement in IGA at Week 4.

Secondary Efficacy Endpoints: Proportion of subjects who achieved an IGA of 0 or 1 at Week 4. Proportion of subjects who achieved an EASI-50 (a 50% reduction from the baseline Eczema Area and Severity Index [EASI]) total score at Week 4. Percent change from baseline to Week 4 in peak pruritus as measured with the 24-hour Peak Pruritus Numeric Rating Scale (NRS)

Exploratory Efficacy Endpoints: Proportion of subjects who achieved an IGA of clear or almost clear with at least a 2-point improvement from baseline at all visits. Proportion of subjects who achieved an IGA of clear or almost clear at all visits. Proportion of subjects who achieved an EASI-50 at all visits. Total score and change from baseline at all visits in IGA. Total score, change, and percent change from baseline at all visits in EASI. Total score, change, and percent change from baseline at all visits in peak pruritus as measured with the 24-hour peak pruritus NRS. Total score, change, and percent change from baseline at all visits in whole body BSA affected.

Safety Endpoint: Frequency and severity of adverse events (AE; local and systemic).

Pharmacokinetic Endpoint: PK analysis for RVT-501 and M11 metabolite at the Week 1 visit in subjects 2 to 11 years old.

Statistical Methods

Analysis Populations: All subjects enrolled in the study who had at least one application of investigational product were included in the Safety Set (SS). This was the population for the safety analyses.

The Full Analysis Set (FAS) consisted of all subjects randomized to treatment who have used at least one application of investigational product and who had a baseline efficacy assessment and at least one post-baseline efficacy assessment. This was the primary population used for the efficacy analyses.

The Per-Protocol Set (PPS) consisted of those members of the FAS who had no major protocol violations, had completed the Double-Blind Phase of the study, and who applied at least 50% of the expected doses through the Week 4 visit. The primary and secondary endpoints were analyzed using the PPS as a sensitivity analysis.

The Open-Label Safety Set (OLSS) consisted of all subjects who entered the Open-Label Extension Phase. This set was used for the analyses of demographics and baseline characteristics, adverse events, and concomitant medication of these subjects.

Efficacy Analyses: The proportion of subjects who achieved an IGA score of 0 or 1 with at least a 2-point improvement from baseline to Week 4 (primary endpoint) was summarized with counts and exact binomial 90% confidence interval (CI) for each treatment group. The treatment difference between RVT-501 and placebo at Week 4 was presented with 90% Wald CI limits for the difference and a 2-sided, 2-group, Cochran-Mantel-Haenszel (CMH) test stratified by randomization factors (baseline IGA and age group) with a 10% significance level was used to assess statistical significance.

A similar approach was performed for the analysis of the secondary endpoints with categorical data. The treatments were compared for the peak pruritus NRS percent change from baseline using a Van Elteren test stratified by the randomization factors.

For hypothesis testing, the last observation carried forward (LOCF) was used for continuous data and non-responder imputation (NRI) was used for categorical data to evaluate the impact of missing data.

The primary and secondary categorical endpoints were analyzed using a Fisher's exact test as sensitivity analysis. Analyses of the exploratory efficacy endpoints are described in Section 9.8.5 of this report.

The efficacy data from the Open-Label Extension Phase were summarized descriptively by initial treatment group and overall.

Pharmacokinetic Analyses: RVT-501 and M11 were measured in plasma by a validated assay. Plasma concentrations were summarized as continuous variables.

Safety Analyses: The number and proportion of subjects with AEs were summarized by treatment, system organ classification, and preferred term for all adverse events, all adverse events considered by the investigator to be related to study drug, all serious adverse events (SAE), all Common Terminology Criteria for Adverse Events (CTCAE) Grade 3 or higher AEs, and all adverse events leading to study discontinuation. Summaries of AEs were presented separately for the Double-Blind and Open-Label Extension Phases.

Laboratory data were analyzed using descriptive summary statistics and changes from baseline. Categorical safety data were analyzed using frequency tables and, if applicable, shift tables. Vital signs were listed by subjects and summarized by treatment.

No formal statistical comparisons were made for safety data.

Interim Analyses: No interim analysis was performed for this study.

Summary of Results

Study Disposition: A total of 110 subjects were enrolled, and 99 subjects completed the Double-Blind Phase. When treatment assignments were unblinded for statistical analysis, a randomization imbalance was discovered. Subject randomization was planned to be 1:1 active RVT-501 0.5% ointment versus vehicle ointment. As a result of the randomization imbalance, 77 subjects received the vehicle and 33 subjects received the active treatment.

Eleven subjects withdrew from the Double-Blind Phase prematurely: five subjects did not complete the Double-Blind Phase due to AEs, two were lost to follow-up, two withdrew consent, one was withdrawn for a protocol deviation, and one for noncompliance with study visit attendance.

Subjects who completed the Double-Blind Phase could elect to enroll in the optional Open-Label Extension Phase. A total of 93 subjects entered the Open-Label Phase. Six subjects who completed the Double-Blind Phase did not enter the Open-Label Extension Phase, either because of an adverse event, a physician decision, a protocol deviation, withdrawal of consent, or other reasons. A total of 84 subjects completed the Open-Label Extension Phase.

All subjects who entered the study were included in the SS (n=110). Two subjects were excluded from the FAS because they did not have post-baseline efficacy data, and 77 subjects in the vehicle group and 31 subjects in the RVT-501 0.5% group were included. Fourteen subjects were excluded from the PPS: two who were not included in the FAS, six were excluded because they used prohibited medications, five were excluded because they did not complete the Double-Blind Phase of the study, and one was excluded because less than 50% of the expected doses were applied. All subjects who entered the Open-Label Phase were included in the OLSS.

Demographic and Baseline Characteristics: The proportion of subjects in the 2 to 11 years subgroup was higher in the RVT-501 0.5% group than in the vehicle group. The mean BSA affected by atopic dermatitis was similar over treatment groups (18.1% in the vehicle group and 17.5% in the RVT-501 0.5% group). Most of the subjects had an IGA of disease severity of 3 (moderate) at baseline. Efficacy Results: The FAS was the primary population used for the efficacy analyses. Improvement in IGA was generally faster and numerically higher in the RVT-501 0.5% group than in the vehicle group. A total of 16.1% of the subjects in the RVT-501 0.5% group achieved an IGA score of clear or almost clear with at least a 2-point improvement from baseline compared to 11.7% of the subjects in the vehicle group after 4 weeks of treatment. The difference between the groups was not statistically significant. Similar results were observed with the secondary endpoint of subjects who achieved an IGA score of clear or almost clear.

The proportion of subjects who achieved an EASI-50 was higher in the RVT-501 0.5% group than in the vehicle group as early as one week after the start of treatment and this was sustained during the Double-Blind Phase. At Week 4, 61.3% of the subjects achieved an EASI-50 in the RVT-501 0.5% group compared to 40.3% in the vehicle group, and the difference between the groups was statistically significant (P=0.053). A similar rapid response was also observed in the percent change from baseline in EASI and BSA affected with atopic dermatitis (AD), one or two weeks, respectively, after the start of the treatment.

At the Week 4 visit, there was a decrease in pruritus of 35.63% with RVT-501 0.5% and 26.34% with the vehicle in peak pruritus with the numeric rating. However, the difference was not statistically significant (P=0.14).

Subgroup analyses by IGA severity at baseline and age group (2 to 11 and 12 to 17 years) did not suggest higher efficacy in a particular subgroup.

In general, the additional 4 weeks of treatment with RVT-501 0.5% in subjects who were already receiving the active ointment did not have a significant impact on atopic dermatitis progression and pruritus severity. After 8 weeks of continuous treatment with RVT-501 0.5%, 18.5% of the subjects achieved an IGA score of clear or almost clear with at least a 2-point improvement, and there was a decrease in the EASI, BSA, and peak pruritus NRS of 42.7%, 53.3%, and 32.4%, respectively. Subjects in the vehicle group who started RVT-501 0.5% at Week 4 achieved similar responses after 4 weeks of treatment when compared to subjects who applied the active ointment from baseline. See Table 24.

TABLE 24
Results

| | | | |
|---|---|---|---|
| | | | P value for |
| | | | difference |
| | Vehicle | RVT-501 0.5% | between the |
| Statistics ^{a} | (N = 77) | (N = 31) | groups ^{b} |

Proportion of Subjects with at Least a 2-Point Improvement in IGA
to Clear or Almost Clear at Week 4

| | | | |
|---|---|---|---|
| N(%) | 9 (11.7) | 5 (16.1) | 0.65 |

Proportion of Subjects with an IGA of Clear or Almost Clear at Week 4

| | | | |
|---|---|---|---|
| N(%) | 14 (18.2) | 5 (16.1) | 0.63 |

Proportion of Subjects who Achieved EASI-50 at Week 4

| | | | |
|---|---|---|---|
| N(%) | 31 (40.3) | 19 (61.3) | 0.053 |

Percent Change from Baseline in EASI at Week 4

| | | | |
|---|---|---|---|
| Mean (SD) | −34.82 (55.588) | −39.85 (85.808) | 0.02 |

Percent Change from Baseline in Total Affected BSA at Week 4

| | | | |
|---|---|---|---|
| Mean(SD) | −31.76 (37.224) | −46.66 (39.895) | 0.03 |

Percent Change from Baseline in Peak Pruritus NRS Score at Week 4

| | | | |
|---|---|---|---|
| Mean(SD) | −26.34 (44.089) | −35.63 (51.219) | 0.14 |

BSA = body surface area; EASI = eczema area and severity index; IGA = investigator's global assessment; NRS = numeric rating scale; SD = standard deviation.
^{a} Subjects with missing data were evaluated using the non-responder imputation or last observation carried forward method.
^{b} Significance level is 0.10.

Pharmacokinetic Results: PK samples were collected on a total of 16 subjects ages 2-11 years. No or minimal systemic absorption was observed for most subjects following topical administration of RVT-501 0.5% ointment to all affected lesions. Three subjects out of 16 had measurable plasma concentrations of RVT-501; two subjects had relatively high concentrations of RVT-501 (one had a value of 306 ng/mL and the other had a value above the upper limit of quantification). These two subjects were 3 years old, had IGA score of 3 (moderate) at baseline, and had BSA and EASI scores at baseline above the overall study average. A total of eight subjects out of 16 had measurable concentrations of the M11 metabolite, all of which were near the lower limit of quantitation.

Safety Results: RVT-501 was generally safe and well tolerated. There were no deaths during this study and four subjects (two in the vehicle group and two in the RVT-501 group) experienced SAE that were all deemed not related to the study treatment by the investigator. Overall, 27 subjects (24.5%) reported at least one adverse event during the Double-Blind Phase of the study, with a total of 42 events reported. Four subjects experienced AEs with severity CTCAE grade 3 (severe) or higher, but only one (application site pruritus) was judged to be related to the study drug and it was experienced by a subject in the vehicle group. There was a higher number of subjects reporting at least one event in the RVT-501 0.5% group (36.4%) than in the vehicle group (19.5%).

A total of 10 subjects (9.1%) reported 11 events at the application site. Five subjects (4.5%) reported application site pruritus (2 subjects [2.6%] in the vehicle group and 3 subjects [9.1%] in the RVT-501 0.5% group). One subject (1.3%) randomized to the vehicle group reported burning after application that was recorded under application site pain. No events of application site stinging were reported.

No treatment-related AEs led to study discontinuation in subjects in the RVT-501 0.5% group. Four subjects (5.2%) of the vehicle group had treatment-related AEs that led to study discontinuation (dermatitis contact, application site pruritus, application dermatitis, and application site pain).

Finally, there were no clinically significant findings in safety laboratory results that resulted in an AE, and no trends detected between treatment groups for the safety laboratory results and vital signs.

Proportion of Subjects Who Achieved an Investigator's Global Assessment of Clear or Almost Clear With at Least a 2-Point Improvement From Baseline: The proportion of subjects that had at least a 2-point improvement from baseline and achieved an IGA of clear or almost clear at Week 4 is presented in Table 25 for the Full Analysis Set. A proportion of 16.1% of the subjects who received RVT-501 0.5% achieved an IGA of clear or almost clear with at least a 2-point improvement from baseline compared to 11.7% of the subjects who received the vehicle. The difference between the groups was not statistically significant (P=0.65).

TABLE 25
Proportion of Subjects Who Achieved an IGA of Clear
or Almost Clear With at Least a 2-Point Improvement
From Baseline at Week 4 (Full Analysis Set)

| | Vehicle | RVT-501 0.5% |
|---|---|---|
| Response | (N = 77) | (N = 31) |
| Subjects with at least a 2-point improvement | 9 (11.7) | 5 (16.1) |
| to clear or almost clear, n (%) ^{a} | | |
| 90% CI ^{b} | 6.2, 19.5 | 6.6, 31.0 |
| Treatment difference (%) | | 4.4 |
| 90% CI ^{c} | | −8.0, 16.9 |
| P value ^{d} | | 0.65 |

CI = confidence interval
^{a} Subjects with missing data were evaluated using the non-responder imputation method.
^{b} 90% CI from an exact binomial test.
^{c} 90% Wald CI.
^{d} Two-sided P value from Cochran-Mantel-Haenszel test stratified by age group and baseline severity.

The proportion of subjects who achieved an IGA of clear or almost clear with at least a 2-point improvement from baseline increased slightly faster in the RVT-501 0.5% group than in the vehicle group between baseline and Week 4, but the difference was not statistically significant (Table 26). The proportion of subjects achieving an IGA of clear or almost clear with at least a 2-point improvement from baseline in the RVT-501 0.5% group was maintained during the 4-week Open-Label Extension Phase. In the vehicle group, an additional 9 subjects (14.2%) reached this endpoint 4 weeks after starting the treatment with RVT-501 0.5% ointment for a total of 18 subjects (28.6%) with such improvement from the baseline visit.

TABLE 26
Proportion of Subjects Who Achieved an IGA of Clear
or Almost Clear With at Least a 2-Point Improvement
From Baseline Over Time (Full Analysis Set)

| Time | Vehicle | RVT-501 0.5% |
|---|---|---|
| Response | (N = 77) | (N = 31) |
| Week 1 | Double-Blind Phase | |
| Subjects with non-missing IGA, n | 75 | 30 |
| Subjects with at least a 2-point improvement | 1 (1.3) | 1 (3.3) |
| to clear or almost clear, n (%) | | |
| 90% CI | 0.1, 6.2 | 0.2, 14.9 |
| Week 2 | | |
| Subjects with non-missing IGA, n | 73 | 31 |
| Subjects with at least a 2-point improvement | 7 (9.6) | 4 (12.9) |
| to clear or almost clear, n (%) | | |
| 90% CI | 4.6, 17.3 | 4.5, 27.1 |
| Week 4 | | |
| Subjects with non-missing IGA, n | 74 | 30 |
| Subjects with at least a 2-point improvement | 9 (12.2) | 5 (16.7) |
| to clear or almost clear, n (%) | | |
| 90% CI | 6.5, 20.3 | 6.8, 31.9 |
| Week 6 | Open-Label Phase | |
| Subjects with non-missing IGA, n | 62 | 29 |
| Subjects with at least a 2-point improvement | 22 (35.5) | 6 (20.7) |
| to clear or almost clear, n (%) | | |
| 90% CI | 25.4, 46.7 | 9.4, 36.8 |
| Week 8 | | |
| Subjects with non-missing IGA, n | 63 | 27 |
| Subjects with at least a 2-point improvement | 18 (28.6) | 5 (18.5) |
| to clear or almost clear, n (%) | | |
| 90% CI | 19.4, 39.4 | 7.6, 35.1 |

CI, confidence interval; IGA = investigator global assessment
Observed cases are presented and 90% CIs are from an exact binomial test.

Proportion of Subjects Who Achieved an Investigator's Global Assessment of Clear or Almost Clear: The proportion of subjects who achieved an IGA of clear or almost clear at Week 4 is presented in Table 27 for the Full Analysis Set. A proportion of 16.1% of the subjects who received RVT-501 0.5% achieved an IGA of clear or almost clear compared to 18.2% of the subjects who received the vehicle. The difference between the groups was not statistically significant (P=0.63).

TABLE 27
Proportion of Subjects Who Achieved an IGA of Clear
or Almost Clear at Week 4 (Full Analysis Set)

| | Vehicle | RVT-501 0.5% |
|---|---|---|
| Response | (N = 77) | (N = 31) |
| Subjects who achieved | 14 (18.2) | 5 (16.1) |
| clear or almost clear, n (%) ^{a} | | |
| 90% CI ^{b} | 11.3, 27.0 | 6.6, 31.0 |
| Treatment difference (%) | | −2.1 |
| 90% CI ^{c} | | −15.1, 11.0 |
| P value ^{d} | | 0.63 |

^{a} Subjects with missing data were evaluated using the non-responder imputation method.
^{b} 90% CI from an exact binomial test.
^{c} 90% Wald CI.
^{d} Two-sided Pvalue from Cochran-Mantel-Haenszel test stratified by age group and baseline severity.

The proportion of subjects who achieved an IGA of clear or almost clear over time was very similar between the groups in the Double-Blind Phase (Table 28). During the 4-week Open-Label Extension Phase, additional subjects (7.4%) achieved an IGA of clear or almost clear in the RVT-501 0.5% group. In the vehicle group, an additional 8 subjects (12.7%) reached this endpoint 4 weeks after starting treatment with RVT-501 0.5% ointment for a total of 22 subjects (34.9%).

TABLE 28
Proportion of Subjects Who Achieved an IGA of Clear
or Almost Clear Over Time (Full Analysis Set)

| | | |
|---|---|---|
| Time | Vehicle | RVT-501 0.5% |
| Response | (N = 77) | (N = 31) |

| | |
|---|---|
| Week 1 | Double-Blind Phase |

| | | |
|---|---|---|
| Subjects with non-missing IGA, n | 75 | 30 |
| Subjects who achieved | 2 (2.7) | 1 (3.3) |
| clear or almost clear, n (%) | | |
| 90% CI | 0.5, 8.2 | 0.2, 14.9 |
| Week 2 | | |
| Subjects with non-missing IGA, n | 73 | 31 |
| Subjects who achieved | 9 (12.3) | 5 (16.1) |
| clear or almost clear, n (%) | | |
| 90% CI | 6.6, 20.5 | 6.6, 31.0 |
| Week 4 | | |
| Subjects with non-missing IGA, n | 74 | 30 |
| Subjects who achieved | 14 (18.9) | 5 (16.7) |
| clear or almost clear, n (%) | | |
| 90% CI | 11.8, 28.0 | 6.8, 31.9 |

| | |
|---|---|
| Week 6 | Open-Label Phase |

| | | |
|---|---|---|
| Subjects with non-missing IGA, n | 62 | 29 |
| Subjects who achieved | 26 (41.9) | 7 (24.1) |
| clear or almost clear, n (%) | | |
| 90% CI | 31.3, 53.2 | 11.9, 40.6 |
| Week 8 | | |
| Subjects with non-missing IGA, n | 63 | 27 |
| Subjects who achieved | 22 (34.9) | 7 (25.9) |
| clear or almost clear, n (%) | | |
| 90% CI | 25.0, 46.0 | 12.9, 43.2 |

CI = confidence interval; IGA = investigator global assessment
Observed cases are presented and 90% CIs are from an exact binomial test.

Change From Baseline in Investigator's Global Assessment Score: Shifts from baseline at Week 4 are presented in Table 29 for the Full Analysis Set. Over time, the proportion of subjects presenting an improvement in IGA scores was more pronounced in the RVT-501 0.5% group than in the vehicle group at every visit. At Week 4, 23 subjects (76.6%) in the RVT-501 0.5% group had an improvement in their IGA scores compared to 39 subjects (52.7%) in the vehicle group. At this visit, only three subjects had a worsening in their IGA scores (2 subjects [2.7%] in the vehicle group and 1 subject [3.3%] in the RVT-501 0.5% group.

TABLE 29
Shift Table From Baseline for IGA at Week 4 (Full Analysis Set)

| | Week 4 Values, n (%) | | | | | |
|---|---|---|---|---|---|---|
| | | Almost | Mild | Moderate | | |
| | Clear | clear | disease | disease | Severe | |
| | (0) | (1) | (2) | (3) | (4) | Total |
| Baseline Values | | | | | | |
| Vehicle (N = 77) | | | | | | |
| Mild disease (2) | 0 (0.0) | 5 (6.8) | 6 (8.1) | 1 (1.4) | 0 (0.0) | 12 (16.2) |
| Moderate disease (3) | 0 (0.0) | 9 (12.2) | 25 (33.8) | 27 (36.5) | 1 (1.4) | 62 (83.8) |
| Total | 0 (0.0) | 14 (18.9) | 31 (41.9) | 28 (37.8) | 1 (1.4) | 74 (100.0) |
| Baseline Values | | | | | | |
| RVT-501 0.5% (N = 31 ) | | | | | | |
| Mild disease (2) | 2 (6.7) | 0 (0.0) | 2 (6.7) | 1 (3.3) | 0 (0.0) | 5 (16.7) |
| Moderate diseased (3) | 0 (0.0) | 3 (10.0) | 18 (60.0) | 4 (13.3) | 0 (0.0) | 25 (83.3) |
| Total | 2 (6.7) | 3 (10.0) | 20 (66.7) | 5 (16.7) | 0 (0.0) | 30 (100.0) |

A summary of the mean IGA scores over time, including mean change from baseline, is provided in Table 30 for the Full Analysis Set.

There was a constant increase in mean change from baseline in IGA over time in the RVT-501 0.5% group during the Double-Blind Phase. Subjects in the vehicle group improved their IGA at a slower rate, but the difference between the groups was not statistically significant. The additional 4 weeks of treatment with RVT-501 0.5% during the Open-Label Extension Phase did not have a significant impact on the IGA of subjects in the RVT-501 0.5% group, as shown by a mean IGA that was maintained until Week 8. Subjects in the vehicle group who entered the Open-Label Phase had a significant improvement in their IGA score after 4 weeks of treatment with RVT-501 0.5% (−1.1 mean change at Week 8 compared to −0.6 at Week 4).

TABLE 30
Summary of IGA Scores Over Time (Full Analysis Set)

| Time Point | Vehicle | | RVT-501 0.5% | |
|---|---|---|---|---|
| Parameter Statistics | (N = 77) | | N = 31) | |
| Baseline | Double-Blind Phase | | | |
| N | 77 | | 31 | |
| Mean (SD) | 2.8 | (0.37) | 2.8 | (0.37) |
| Median | 3.0 | | 3.0 | |
| Min, max | 2, 3 | | 2, 3 | |
| IQR | 3.0-3.0 | | 3.0-3.0 | |
| Week 1 | | | | |
| N | 75 | | 30 | |
| Mean (SD) | 2.6 | (0.57) | 2.3 | (0.65) |
| Median | 3.0 | | 2.0 | |
| Min, max | 1, 4 | | 0, 3 | |
| IQR | 2.0-3.0 | | 2.0-3.0 | |
| Mean change | −0.3 | (−0.4, −0.2) | −0.5 | (−0.7, −0.4) |
| from baseline (90% CI) | | | | |
| Week 2 | | | | |
| N | 73 | | 31 | |
| Mean (SD) | 2.3 | (0.70) | 2.0 | (0.71) |
| Median | 2.0 | | 2.0 | |
| Min, mam | 1, 4 | | 0, 3 | |
| IQR | 2.0-3.0 | | 2.0-2.0 | |
| Mean change | −0.5 | (−0.7, −0.4) | −0.8 | (−1.0, −0.6) |
| from baseline (90% CI) | | | | |
| Week 4 | | | | |
| N | 74 | | 30 | |
| Mean (SD) | 2.2 | (0.76) | 1.9 | (0.74) |
| Median | 2.0 | | 2.0 | |
| Min, max | 1, 4 | | 0, 3 | |
| IQR | 2.0-3.0 | | 2.0-2.0 | |
| Mean change | −0.6 | (−0.8, −0.5) | −0.9 | (−1.1, −0.7) |
| from baseline (90% CI) | | | | |
| Week 6 | Open-Label Phase | | | |
| N | 62 | | 29 | |
| Mean (SD) | 1.8 | (0.96) | 2.0 | (0.78) |
| Median | 2.0 | | 2.0 | |
| Min, max | 0, 4 | | 0, 3 | |
| IQR | 1.0-2.0 | | 2.0-2.0 | |
| Mean change | −1.1 | (−1.3, −0.9) | −0.9 | (−1.1, −0.6) |
| from baseline (90% CI) | | | | |
| Week 8 | | | | |
| N | 63 | | 27 | |
| Mean (SD) | 1.8 | (0.91) | 1.9 | (0.87) |
| Median | 2.0 | | 2.0 | |
| Min, max | 0, 4 | | 0, 3 | |
| IQR | 1.0-2.0 | | 1.0-3.0 | |
| Mean change | −1.1 | (−1.3, −0.9) | −0.9 | (−1.2, −0.6) |
| from baseline (90% CI) | | | | |

CI = confidence interval; IQR = interquartile range; SD = standard deviation

Proportion of Subjects Who Achieved a 50% Reduction From Baseline in Eczema Area and Severity Index: The proportion of subjects who achieved a 50% reduction from baseline (EASI-50) at Week 4 is presented in Table 31 for the Full Analysis Set. There was a statistically significant difference between the proportion of subjects who achieved an EASI-50 in the RVT-501 0.5% group (61.3%) and the vehicle group (40.3%) (P=0.053).

TABLE 31
Proportion of Subjects Who Achieved
EASI-50 at Week 4 (Full Analysis Set)

| | Vehicle | RVT-501 0.5% |
|---|---|---|
| Response | (N = 77) | (N = 31) |
| Subjects who achieved EASI-50, n (%) ^{a} | 31 (40.3) | 19 (61.3) |
| 90% CI ^{b} | 30.8, 50.3 | 45.0, 75.9 |
| Treatment difference (%) | | 21.0 |
| 90% CI ^{c} | | 4.0, 38.1 |
| P value ^{d} | | 0.053 |

CI = confidence interval; EASI-50 = 50% reduction from baseline in Eczema Area and Severity Index (EASI)
^{a} Subjects with missing data were evaluated using the non-responder imputation method.
^{b} 90% CI from an exact binomial test.
^{c} 90% Wald CI.
^{d} Two-sided P value from Cochran-Mantel-Haenszel test stratified by age group and baseline severity.

The proportion of subjects who achieved an EASI-50 was higher in the RVT-501 0.5% group than in the vehicle group at all visits between baseline and Week 4 (Table 32). The proportion of subjects who achieved an EASI-50 in the RVT-501 0.5% group did not significantly increase during the 4-week Open-Label Extension Phase. In the vehicle group, an additional 12 subjects (19.0%) reached this endpoint 4 weeks after starting treatment with RVT-501 0.5% ointment for a total of 43 subjects (68.3%) with such improvement from the baseline visit. At Week 8, a similar proportion of subjects had reached an EASI-50 in both treatment groups.

TABLE 32
Proportion of Subjects Who Achieved
EASI-50 Over Time (Full Analysis Set)

| Time | Vehicle | RVT-501 0.5% |
|---|---|---|
| Response | (N = 77) | (N = 31) |
| Week 1 | Double-Blind Phase | |
| Subjects with non-missing EASI, n | 75 | 30 |
| Subjects who achieved EASI-50, n (%) | 6 (8.0) | 8 (26.7) |
| 90% CI | 3.5, 15.2 | 14.0, 43.0 |
| Week 2 | | |
| Subjects with non-missing EASI, n | 73 | 31 |
| Subjects who achieved EASI-50, n (%) | 21 (28.8) | 18 (58.1) |
| 90% CI | 20.2, 38.7 | 41.8, 73.1 |
| Week 4 | | |
| Subjects with non-missing EASI, n | 74 | 30 |
| Subjects who achieved EASI-50, n (%) | 31 (41.9) | 19 (63.3) |
| 90% CI | 32.2, 52.1 | 46.7, 77.9 |
| Week 6 | Open-Label Phase | |
| Subjects with non-missing EASI, n | 62 | 29 |
| Subjects who achieved EASI-50, n (%) | 45 (72.6) | 17 (58.6) |
| 90% CI | 61.8, 81.7 | 41.8, 74.1 |
| Week 8 | | |
| Subjects with non-missing EASI, n | 63 | 27 |
| Subjects who achieved EASI-50, n (%) | 43 (68.3) | 18 (66.7) |
| 90% CI | 57.3, 77.9 | 49.1, 81.4 |

CI = confidence interval; EASI = Eczema Area and Severity Index, EASI-50 = 50% reduction from EASI
Observed cases are presented and 90% CIs are from an exact binomial test.

Change From Baseline in Eczema Area and Severity Index: Mean percent change from baseline in total EASI scores at Week 4 are presented in Table 33 for the Full Analysis Set. At Week 4, the mean percent change from baseline in total EASI score was statistically significantly greater in the RVT-501 0.5% group (−39.85%) than in the vehicle group (−34.82%) (P=0.02).

TABLE 33
Percent Change From Baseline in EASI
at Week 4 (Full Analysis Set)

| | Vehicle | RVT-501 0.5% |
|---|---|---|
| Response | (N = 77) | (N = 31) |
| N ^{a} | 77 | 31 |
| Mean (SD) | −34.82 (55.588) | −39.85 (85.808) |
| Median | −42.11 | −58.97 |
| Min, max | −95.5, 350.0 | −100.0, 342.9 |
| IQR | −67.65 to −10.53 | −80.00 to −35.37 |
| 90% CI | −45.37, −24.28 | −66.00, −13.69 |
| Treatment difference | | −5.02 |
| P value ^{b} | | 0.02 |

CI = confidence interval; IQR = interquartile range; SD = standard deviation
^{a} Subjects with missing data were evaluated using the last observation carried forward method.
^{b} P value from Van Elteren test stratified by the age group and baseline severity.

A summary of EASI scores over time, including change and percent change from baseline, is provided in Table 34 for the Full Analysis Set. The improvement in EASI was greater in the RVT-501 0.5% group after 2 weeks of treatment compared to the vehicle group, but the difference was reduced at Week 4. The mean change from baseline at Week 4 showed a greater change for the RVT-501 0.5% group; however, the mean percent change from baseline was similar for both groups. The EASI scores of subjects in the RVT-501 0.5% group did not significantly improve during the 4-week Open-Label Extension Phase. Subjects in the vehicle group who entered the Open-Label Phase had a significant improvement in their EASI score after 4 weeks of treatment with RVT-501 0.5% (−56% change at Week 8 compared to −35% at Week 4).

TABLE 34
Summary of EASI Scores Over Time (Full Analysis Set)

| Time Point | Vehicle | | RVT-501 0.5% | |
|---|---|---|---|---|
| Parameter Statistics | (N = 77) | | (N = 31) | |
| Baseline | Double-Blind Phase | | | |
| N | 77 | | 31 | |
| Mean (SD) | 10.56 | (5.765) | 11.92 | (7.655) |
| Median | 8.30 | | 11.00 | |
| Min, max | 1.2, 24.4 | | 1.2, 32.9 | |
| IQR | 6.00-15.40 | | 5.90-16.20 | |
| Week 1 | | | | |
| N | 75 | | 30 | |
| Mean (SD) | 8.70 | (5.788) | 7.37 | (5.571) |
| Median | 7.60 | | 5.85 | |
| Min, max | 0.5, 27.8 | | 0.0, 25.1 | |
| IQR | 4.20-12.70 | | 3.40-10.00 | |
| Mean change | −2.01 | (−2.57, −1.44) | −4.45 | (−5.86, −3.04) |
| from baseline (90% CI) | | | | |
| Mean percent change | −19.9 | (−24.7, −15.1) | −35.5 | (−43.6, −27.3) |
| from baseline (90% CI) | | | | |
| Week 2 | | | | |
| N | 73 | | 31 | |
| Mean (SD) | 7.33 | (5.754) | 5.55 | (4.958) |
| Median | 5.80 | | 4.60 | |
| Min, max | 0.3, 23.4 | | 0.0, 26.3 | |
| IQR | 2.90-9.40 | | 2.70-7.40 | |
| Mean change | −3.06 | (−3.73, −2.40) | −6.37 | (−8.05, −4.69) |
| from baseline (90% CI) | | | | |
| Mean percent change | −32.3 | (−39.1, −25.4) | −49.1 | (−60.0, −38.2) |
| from baseline (90% CI) | | | | |
| Week 4 | | | | |
| N | 74 | | 30 | |
| Mean (SD) | 6.80 | (6.062) | 4.70 | (3.185) |
| Median | 5.20 | | 4.65 | |
| Min, max | 0.2, 26.8 | | 0.0, 11.2 | |
| IQR | 2.20-8.80 | | 2.00-6.40 | |
| Mean change | −3.55 | (−4.37, −2.73) | −7.13 | (−9.31, −4.94) |
| from baseline (90% CI) | | | | |
| Mean percent change | −34.5 | (−45.4, −23.7) | −38.4 | (−65.4, −11.5) |
| from baseline (90% CI) | | | | |
| Week 6 | Open-Label Phase | | | |
| N | 62 | | 29 | |
| Mean (SD) | 4.66 | (5.378) | 5.12 | (5.639) |
| Median | 2.65 | | 3.10 | |
| Min, max | 0.0, 23.9 | | 0.0, 25.8 | |
| IQR | 1.20-5.50 | | 1.80-7.20 | |
| Mean change | −5.34 | (−6.18, −4.50) | −5.98 | (−7.89, −4.06) |
| from baseline (90% CI) | | | | |
| Mean percent change | −59.1 | (−66.0, −52.2) | −44.3 | (−62.4, −26.2) |
| from baseline (90% CI) | | | | |
| Week 8 | | | | |
| N | 63 | | 27 | |
| Mean (SD) | 5.23 | (8.025) | 4.89 | (5.753) |
| Median | 2.00 | | 2.90 | |
| Min, max | 0.0, 50.8 | | 0.0, 21.7 | |
| IQR | 0.90-6.70 | | 1.10-5.00 | |
| Mean change | −4.69 | (−6.01, −3.37) | −6.08 | (−8.53, −3.635) |
| from baseline (90% CI) | | | | |
| Mean percent change | −55.9 | (−65.2, −46.7) | −42.7 | (−71.6, −13.8) |
| from baseline (90% CI) | | | | |

CI = confidence interval;
IQR = interquartile range;
SD = standard deviation

Change from Baseline in Total Affected Body Surface Area: Mean percent change from baseline in total affected BSA at Week 4 are presented in Table 35 for the Full Analysis Set. At Week 4, the mean percent change from baseline in total affected BSA was statistically significantly greater in the RVT-501 0.5% group (−46.66%) than in the vehicle group (−31.76%) (P=0.03).

TABLE 35
Percent Change From Baseline in Total Affected
BSA at Week 4 (Full Analysis Set)

| | Vehicle | RVT-501 0.5% |
|---|---|---|
| Response | (N = 77) | (N = 31) |
| N ^{a} | 77 | 31 |
| Mean (BD) | −31.76 (37.224) | −46.66 (39.895) |
| Median | −31.43 | −54.05 |
| Min, Max | −94.2, 99.3 | −100.0, 80.0 |
| IQR | −57.49 to −9.50 | −75.35 to −25.00 |
| 90% CI | −38.82, −24.69 | −58.82, −34.50 |
| Treatment difference | | −14.90 |
| P value ^{b} | | 0.03 |

CI = confidence interval;
IQR = interquartile range;
SD = standard deviation
^{a} Subjects with missing data were evaluated using the last observation carried forward method.
^{b} P value from Van Elteren test stratified by the age group and baseline severity.

A summary of total BSA scores over time, including mean change and percent change from baseline, is provided in Table 36 for the Full Analysis Set. The improvement in BSA was significantly faster in the RVT-501 0.5% group after 2 weeks of treatment compared to the vehicle group, but the difference between the groups was less pronounced at Week 4. The BSA of subjects in the RVT-501 0.5% group did not significantly improve during the Open-Label Extension Phase. Subjects in the vehicle group who entered the Open-Label Phase had a significant improvement in their affected BSA after 4 weeks of treatment with RVT-501 0.5% (−56% change at Week 8 compared to −32% at Week 4). At Week 8, mean percent change in affected BSA was similar in both treatment groups.

TABLE 36
Summary of Total Affected BSA Over Time (Full Analysis Set)

| Time Point | Vehicle | | RVT-501 0.5% | |
|---|---|---|---|---|
| Parameter Statistics | (N = 77) | | (N = 31) | |
| Baseline | Double-Blind Phase | | | |
| N | 77 | | 31 | |
| Mean (SD) | 18.05 | (10.970) | 17.48 | (11.118) |
| Median | 15.10 | | 15.60 | |
| Min, max | 5.0, 40.0 | | 5.0, 40.0 | |
| IQR | 8.00-25.10 | | 7.10-26.50 | |
| Week 1 | | | | |
| N | 75 | | 30 | |
| Mean (SD) | 15.34 | (10.482) | 13.58 | (9.451) |
| Median | 11.80 | | 10.80 | |
| Min, max | 2.0, 42.7 | | 0.0, 40.0 | |
| IQR | 6.60-22.00 | | 6.30-19.90 | |
| Mean change | −2.53 | (−3.36, −1.70) | −3.97 | (−5.84, −2.10) |
| from baseline (90% CI) | | | | |
| Mean percent change | −14.8 | (−19.1, −10.6) | −20.3 | (−28.6, −12.0) |
| from baseline (90% CI) | | | | |
| Week 2 | | | | |
| N | 73 | | 31 | |
| Mean (SD) | 13.72 | (11.166) | 9.60 | (7.820) |
| Median | 10.00 | | 7.00 | |
| Min, max | 1.0, 51.5 | | 0.0, 40.0 | |
| IQR | 5.60-20.00 | | 5.80-13.60 | |
| Mean change | −3.52 | (−4.77, −2.28) | −7.88 | (−10.21, −5.56) |
| from baseline (90% CI) | | | | |
| Mean percent change | −24.8 | (−30.9, −18.8) | −40.8 | (−51.1, −30.5) |
| from baseline (90% CI) | | | | |
| Week 4 | | | | |
| N | 74 | | 30 | |
| Mean (SD) | 12.44 | (11.512) | 7.97 | (5.801) |
| Median | 9.20 | | 7.00 | |
| Min, max | 0.5, 53.2 | | 0.0, 19.0 | |
| IQR | 4.00-17.50 | | 3.30-13.00 | |
| Mean change | −4.75 | (−6.26, −3.23) | −9.58 | (−12.58, −6.59) |
| from baseline (90% CI) | | | | |
| Mean percent change | −31.6 | (−39.0, −24,3) | −45.4 | (−57.8, −33.0) |
| from baseline (90% CI) | | | | |
| Week 6 | Open-Label Phase | | | |
| N | 62 | | 29 | |
| Mean (SD) | 9.59 | (13.667) | 8.10 | (11.241) |
| Median | 4.85 | | 4.40 | |
| Min, max | 0.0, 81.2 | | 0.0, 58.9 | |
| IQR | 1.20-12.00 | | 2.20-8.00 | |
| Mean change | −6.66 | (−8.90, −4.42) | −8.67 | (−12.26, −5.09) |
| from baseline (90% CI) | | | | |
| Mean percent change | −52.8 | (−62.8, −42.9) | −49.3 | (−62.9, −35.6) |
| from baseline (90% CI) | | | | |
| Week 8 | | | | |
| N | 63 | | 27 | |
| Mean (SD) | 8.91 | (12.968) | 7.54 | (7.728) |
| Median | 4.00 | | 4.50 | |
| Min, max | 0.0, 80.0 | | 0.0,27.4 | |
| IQR | 1.00-10.60 | | 2.70-10.00 | |
| Mean change | −7.20 | (−9.23, −5.16) | −9.61 | (−12.84, −6.38) |
| from baseline (90% CI) | | | | |
| Mean percent change | −55.9 | (−64.8, −47.0) | −53.3 | (−65.4, −41.1) |
| from baseline (90% CI) | | | | |

CI = confidence interval;
IQR = interquartile range;
SD = standard deviation

Change From Baseline in Peak Pruritus Numeric Rating Scale Score: The peak pruritus NRS asks the subject to rate the peak severity of his/her itch from “no itch (0),” to “worst itch possible (10)” over a 24-hour period. Mean percent changes from baseline in peak pruritus NRS score at Week 4 are presented in Table 37 for the Full Analysis Set.

At Week 4, the mean percent change from baseline in peak pruritus NRS score was not statistically different between the RVT-501 0.5% group (−35.63%) and the vehicle group (−26.34%) (P=0.14).

TABLE 37
Percent Change From Baseline in Peak Pruritus
NRS Score at Week 4 (Full Analysis Set)

| | Vehicle | RVT-501 0.5% |
|---|---|---|
| Response | (N = 77) | (N = 31) |
| N ^{a, b} | 76 | 31 |
| Mean (SD) | −26.34 (44.089) | −35.63 (51.219) |
| Median | −24.29 | −42.86 |
| Min, max | −100.0, 150.0 | −100.0, 133.3 |
| IQR | −50.00 to 0.00 | −60.00 to −11.11 |
| 90% CI | −34.76, −17.92 | −51.24, −20.02 |
| Treatment difference | | −9.29 |
| P value ^{c} | | 0.14 |

CI = confidence interval;
IQR = interquartile range;
SD = standard deviation
^{a} Subjects with missing data were evaluated using the last observation carried forward method.
^{b} Subjects with a score of zero at baseline and non-zero at Week 4 were excluded because percent cannot be determined.
^{c} P value from Van Elteren test stratified by the age group and baseline severity.

A summary of peak pruritus NRS score over time, including mean change and percent change from baseline, is provided in Table 38 for the Full Analysis Set. The improvement in peak pruritus was faster in the RVT-501 0.5% group after 2 weeks of treatment compared to the vehicle group. The peak pruritus NRS score in the RVT-501 0.5% group did not improve during the 4-week Open-Label Extension Phase. Subjects in the vehicle group who entered the Open-Label Phase had a significant improvement in their peak pruritus NRS score after 4 weeks of treatment with RVT-501 0.5% (−45% change at Week 8 compared to −27% oat Week 4).

TABLE 38
Summary of Peak Pruritus NRS Score
Over Time (Full Analysis Set)

| Time Point | Vehicle | | RVT-501 0.5% | |
|---|---|---|---|---|
| Parameter Statistics | (N = 77) | | (N = 31) | |
| Baseline | Double-Blind Phase | | | |
| N | 77 | | 31 | |
| Mean (SD) | 6.4 | (2.37) | 6.0 | (2.19) |
| Median | 7.0 | | 6.0 | |
| Min, max | 0, 10 | | 1, 10 | |
| IQR | 5.0-8.0 | | 5.0-7.0 | |
| Week 1 | | | | |
| N | 75 | | 30 | |
| Mean (SD) | 5.4 | (2.69) | 4.3 | (2.02) |
| Median | 5.0 | | 4.0 | |
| Min, max | 0, 10 | | 0, 10 | |
| IQR | 3.0-8.0 | | 3.0-6.0 | |
| Mean change | −1.0 | (−1.5, −0.6) | −1.6 | (−2.2, −1.1) |
| from baseline (90% CI) | | | | |
| Mean percent change | −15.9 | (−24.7, −7.1) | −24.3 | (−33.0, −15.7) |
| from baseline (90% CI) ^{a} | | | | |
| Week 2 | | | | |
| N | 73 | | 31 | |
| Mean (SD) | 4.8 | (2.52) | 3.6 | (2.21) |
| Median | 5.0 | | 4.0 | |
| Min, max | 0, 10 | | 0, 9 | |
| IQR | 3.0-7.0 | | 2.0-5.0 | |
| Mean change | −1.7 | (−2.1, −1.2) | −2.4 | (−3.1, −1.6) |
| from baseline (90% CI) | | | | |
| Mean percent change | −25.6 | (−32.0, −19.1) | −36.4 | (−47.0, −25.9) |
| from baseline (90% CI) ^{a} | | | | |
| Week 4 | | | | |
| N | 74 | | 30 | |
| Mean (SD) | 4.5 | (2.64) | 3.5 | (2.21) |
| Median | 5.0 | | 4.0 | |
| Min, max | 0, 10 | | 0, 7 | |
| IQR | 2.0-6.0 | | 2.0-5.0 | |
| Mean change | −1.9 | (−2.4, −1.4) | −2.4 | (−3.2, −1.6) |
| from baseline (90% CI) | | | | |
| Mean percent change | −26.5 | (−35.1 −17.9) | −36.4 | (−52.5, −20.4) |
| from baseline (90% CI) ^{a} | | | | |
| Week 6 | Open-Label Phase | | | |
| N | 62 | | 29 | |
| Mean (SD) | 3.6 | (2.57) | 3.7 | (2.25) |
| Median | 3.5 | | 4.0 | |
| Min, max | 0, 10 | | 0, 9 | |
| IQR | 2.0-5.0 | | 2.0-5.0 | |
| Mean change | −2.9 | (−3.4, −2.3) | −2.2 | (−3.2, −1.3) |
| from baseline (90% CI) | | | | |
| Mean percent change | −41.7 | (−49.7, −33.6) | −30.8 | (−49.4, −12.2) |
| from baseline (90% CI) ^{a} | | | | |
| Week 8 | | | | |
| N | 63 | | 27 | |
| Mean (SD) | 3.4 | (2.72) | 3.6 | (2.45) |
| Median | 3.0 | | 3.0 | |
| Min, max | 0, 10 | | 0, 9 | |
| IQR | 1.0-5.0 | | 2.0-6.0 | |
| Mean change | −3.0 | (−3.6 to −2.4) | −2.3 | (−3.2, −1.4) |
| from baseline (90% CI) | | | | |
| Mean percent change | −44.9 | (−53.4, −36.5) | −32.4 | (−48.6, −16.3) |
| from baseline (90% CI) ^{a} | | | | |

CI = confidence interval;
IQR interquartile range;
SD = standard deviation
^{a} Subjects with a score of zero at baseline and non-zero at the timepoint were excluded because percent change cannot be determined.

Pharmacokinetic Concentration Results: A single blood sample was collected pre-dose in subjects aged 2 to 11 years old at Week 1 to assess the concentration of RVT-501 and the M11 metabolite in plasma.

A summary of plasma concentrations of RVT-501 and M11 metabolite is shown in Table 39 for the Full Analysis Set. Measurable concentrations of RVT-501 were reported in three subjects (Subject 03001 [1.07 ng/mL], Subject 05002 [306.00 ng/mL], and Subject 21001 [above upper limit of quantitation]) (lower limit of quantitation=0.25 ng/mL).

Subject 03001 was 4 years old, had an IGA score of 3 (moderate), a total EASI of 14.1, and a BSA affected by AD of 25.0% at baseline. The morning application of the study product was performed approximately 9.5 hours before the PK sample collection.

Subject 05002 was 3 years old, had an IGA score of 3 (moderate), a total EASI of 13.2, and a BSA affected by AD of 28.4% at baseline. The last application before the PK sample collection was performed in the evening prior to the day of the Week 1 visit.

Subject 21001 was 3 years old, had an IGA score of 3 (moderate), a total EASI of 20.0, and a BSA affected by AD of 26.5% at baseline. The morning application of the study product was performed approximately 9.5 hours before the PK sample collection.

Measurable concentrations of plasma M11 were reported in eight subjects. The highest concentration measured was 16.90 ng/mL in Subject 05002. These subjects had an IGA score of 3 (moderate), a total EASI between 3.4 and 28.5, and a BSA affected by AD between 9.0% and 37.0% at baseline.

TABLE 39
Summary of Plasma Concentration of RVT-501 and M11 Metabolite
at Week 1 in Subjects Aged 2 to 11 Years Old Following Twice
Daily Application of RVT-501 0.5% (Full Analysis Set)

| Statistics | | |
|---|---|---|
| (ng/mL) | RVT-501 | M11 |
| N | 16 | 16 |
| N with concentrations | 3 | 8 |
| above LLQ | | |
| Mean (SD) | 31.69 (88.531) | 1.50 (4.194) |
| Median | 0 | 0.19 |
| Min, max | 0, 306.00 | 0, 16.90 |
| IQR | 0.0-0.0 | 0.0-0.7 |

IQR = interquartile range;
SD = standard deviation;
LLQ = lower limit of quantitation

Discussion

The objectives of this study were to confirm the efficacy observed in study RVT-501-2001 in pediatric subjects with atopic dermatitis and investigate if there is a difference in response between the adult and pediatric populations. Safety and pharmacokinetics of the drug were also assessed as secondary objectives.

A total of 110 pediatric subjects with mild to moderate atopic dermatitis were randomized in the study. At the end of the study, when the treatment assignments were unblinded for statistical analysis, a randomization imbalance was discovered. Subject randomization was planned to be 1:1 active RVT-501 0.5% ointment versus vehicle ointment. As a result of the randomization imbalance, 77 subjects received vehicle and 33 subjects received active treatment. The most probable cause identified was the apparent lack of clear understanding between the RT vendor and the statistics vendor in regards to the randomization format to be provided according to the Veracity Logic VLIRT® system functionalities. In consequence, the randomization codes were assigned by the IRT vendor based on lowest available randomization code in each protocol defined stratum (subject age and disease severity), which was not how the statistic vendor built the randomization list.

The mean affected BSA was similar over the treatment groups at baseline (18.1% in the vehicle group and 17.5% in the RVT-501 0.5% group). Most of the subjects (84.3%) had an IGA of disease severity of 3 (moderate) at baseline. The proportion of subjects based on baseline IGA severity was similar in both treatment groups despite the randomization imbalance. The mean age was similar in both treatment groups. However, there was a higher proportion of subjects in the 2 to 11 years subgroup in the RVT-501 0.5% group (46.8% in the vehicle group and 60.6% in the RVT-501 0.5% group) and a higher proportion of subjects in the 12 to 17 years subgroup in the vehicle group (53.2% in the vehicle group and 39.4% in the RVT-501 0.5% group). The difference in the proportion of age group despite the planned stratification by this factor is likely due to the imbalance randomization that was discovered when the study was unblinded. Additional analyses performed by the sponsor suggested that this imbalance likely had minimal impact on the efficacy data. The majority of subjects were female and the proportion of Black or African American was higher in the vehicle group.

Results of this Phase 2 study suggest that RVT-501 0.5% provided a modest benefit versus the vehicle ointment. The improvement in IGA was generally faster and numerically higher in the RVT-501 0.5% group than in the vehicle group. A total of 16.1% of the subjects in the RVT-501 0.5% group achieved an IGA score of clear or almost clear with at least a 2-point improvement compared to 11.7% of the subjects in the vehicle group after 4 weeks of treatment. The difference between the groups was not statistically significant. The response was lower than the response observed in a previous study (RVT-501-2001), where 31.6% of the adolescent subjects achieved this endpoint after 4 weeks of treatment with RVT-501 0.5% versus 9.1% of the vehicle-treated subjects. Similar results were observed with the secondary endpoint of subjects who achieved an IGA score of clear or almost clear.

Statistical significance was reached at Week 4 for the secondary endpoints of proportion of subjects achieving EASI-50, percent change in EASI, and percent change in BSA. The proportion of subjects who achieved an EASI-50 was higher in the RVT-501 0.5% group than in the vehicle group as early as one week after the start of the treatment, and this was maintained until Week 4. A similar rapid response was also observed in the percent change from baseline in EASI and BSA one or two weeks, respectively, after the start of the treatment.

At the Week 4 visit, there was a decrease of 35.63% with RVT-501 0.5% and 26.34% with the vehicle in peak pruritus with the numeric rating scale. However, this was not statistically significant.

Subgroup analyses by IGA severity at baseline and age group (2 to 11 and 12 to 17 years) did not suggest higher efficacy in a particular subgroup. Only one subject (8.3%) in the 12 to 17 age group achieved an IGA score of 0 or 1 with at least a 2-point improvement from baseline compared to 31.6% of the adolescents in the previous study.

Overall, efficacy results obtained in this study showed lower efficacy as compared to vehicle than what was observed for adolescents in the previous study RVT-501-2001.

After having completed the Double-Blind Phase of the study, subjects could elect to enter a 4-week Open-Label Extension Phase. A total of 93 subjects entered the Open-Label Phase. In general, the additional 4 weeks of treatment with RVT-501 0.5% in subjects who were already receiving the active ointment did not have a significant impact on atopic dermatitis progression and pruritus severity. Subjects in the vehicle group who started RVT-501 0.5% at Week 4 achieved similar responses after 4 weeks when compared to subjects who applied the active ointment from the baseline.

Plasma concentrations of RVT-501 and the M11 metabolite were quantified in 16 subjects aged 2 to 11 years old. Consistent with other studies, no or minimal systemic absorption was observed for most subjects following topical administration of RVT-501 0.5% ointment to all affected lesions. Three subjects (20%) had measurable plasma concentrations of RVT-501; two subjects had relatively high concentrations of RVT-501 (one had a value of 306 ng/mL and the other had a value above the upper limit of quantification). These two subjects were 3 years old, had IGA score of 3 (moderate) at baseline, and their BSA and EASI scores at baseline were above the overall study average. A total of 8 subjects (50%) had measurable concentrations of the M11 metabolite, all of which were near the lower limit of quantitation. This proportion is significantly higher than in the previous study where only 3% of the adolescent and adult subjects had a measurable concentration of the metabolite. Although the bioanalytical method used in this study was more sensitive than in the previous study RVT-501-2001, it may suggest that absorption of RVT-501 is greater in subjects 2 to 11 years old than in adolescents or adults, but that the plasma concentration remains minimal.

RVT-501 0.5% ointment was generally safe and well tolerated. Four subjects experienced SAEs that were all deemed not related to the study drug. There was a higher number of subjects reporting at least one event in the RVT-501 0.5% group (36.4%) than in the vehicle group (19.5%), but frequencies of treatment-related events were similar. A small number of events was reported for each term, including application site reactions.

Ten subjects (9.1%) reported application site reactions; 5 of which (4.5%) reported application site pruritus. Other application site reactions (urticaria, dermatitis, pain) were reported by only one subject including burning after application that was recorded under the application site pain preferred term. No events of application site stinging were reported.

Six subjects (5.5%) had AEs that were considered related to the study drug during the Double-Blind Phase and none during the Open-Label Phase. All these AEs were skin-related. Two subjects (6.1%) in the RVT-501 group had mild application site pruritus that resolved before the end of the study. In the vehicle group, severe application site pruritus, mild application site dermatitis, moderate application site pain, and moderate contact dermatitis were each reported by one subject (1.3%). No trends were detected between the groups in treatment-related AE. Finally, there were no clinically significant findings in safety laboratory results that resulted in an AE, and no trends detected between treatment groups for the safety laboratory results and vital signs.

Conclusions: In this study, RVT-501 0.5% appears to provide a modest clinical benefit in pediatric subjects with mild to moderate atopic dermatitis versus the vehicle.

The improvement in IGA was generally faster and numerically higher in the RVT-501 0.5% group than in the vehicle group. A total of 16.1% of the subjects in the RVT-501 0.5% group achieved an IGA score of clear or almost clear with at least a 2-point improvement from baseline compared to 11.7% in the vehicle group after 4 weeks of treatment. The difference between the groups was not statistically significant.

There was a statistically significant difference between the groups in the proportion of subjects who achieved an EASI-50, in the percent change in EASI, and in the percent change in BSA after 4 weeks of treatment.

Improvements in pruritus were reported in both groups (35.63% decrease in the RVT-501 group versus 26.34% decrease in the vehicle group), but results did not reach statistical significance.

Only three subjects had a detectable level of RVT-501 and eight subjects had a measurable concentration of the active M11 metabolite after 2 weeks of treatment, demonstrating minimal systemic absorption.

Further analyses demonstrated that the impact of the randomization imbalance on the overall efficacy achieved with RVT-501 0.5% ointment was likely minimal.

RVT-501 0.5% ointment was generally safe and well tolerated in pediatric subjects with mild to moderate atopic dermatitis. Five SAEs, all evaluated as not related to study treatment, were observed. The number of subjects who reported application site AEs such as pruritus was low and similar for both groups. One subject (vehicle) reported application site burning sensation and no subjects reported application site stinging.

### Example 7: Open-Label Study to Evaluate the Safety, Tolerability, and Pharmacokinetics of RVT-501 Topical Ointment in Pediatric Patients with Atopic Dermatitis

Study design: Multicenter, open-label, safety, tolerability, and pharmacokinetic study. The study consisted of three phases: Screening (up to 30 days), Treatment Phase (28 days), and Follow-up (7-10 days).

Objectives: Primary: To evaluate the safety and pharmacokinetics (PK) of topical RVT-501 in pediatric subjects with extensive atopic dermatitis.

Secondary: To assess the efficacy of topical RVT-501 in pediatric subjects with extensive atopic dermatitis.

Study design/Methodology: This was a multicenter, open-label, Phase 1b study to evaluate the safety, tolerability, and PK of RVT-501 ointment in pediatric subjects with atopic dermatitis.

Subjects underwent screening procedures within 30 days of enrollment to confirm eligibility. At Day 0 (baseline), while under the supervision of site personnel in the clinic, eligible subjects and their parent(s) or caregiver were instructed on how to apply RVT-501. Study medication was dispensed to subjects and was applied at home as instructed by site personnel between clinic visits.

During the Treatment Phase, subjects, their parent(s), or caregiver applied RVT-501 0.5% ointment to affected areas twice daily for 28 days. Subjects returned to the clinic at Week 1, Week 4, and follow-up for study assessments. On Day 1 and Weeks 2 and 3, subjects were contacted by phone to confirm their status, including any adverse events (AEs) and changes in concomitant medications.

Subjects/caregivers liberally applied sufficient study medication to completely cover each lesion with a thin layer of medication. Medication was applied to all affected areas, including newly appearing lesions and lesions that improved during the study.

There was a follow-up visit 7 (±2) days following the end of study treatment. A subject's total participation in the study lasted up to 10 weeks and included five clinic visits.

Target Population: Approximately 24 evaluable subjects with extensive atopic dermatitis aged 2 to 11 years, with approximately equal distribution across both age groups (ages 2 to 6 and ages 7 to 11) were to be enrolled in this study.

Main Criteria for Inclusion: Male and female pediatric subjects aged 2 to 11 with confirmed diagnosis of atopic dermatitis by Hanifin and Rajka criteria. Subjects with atopic dermatitis covering >25% of the Body Surface Area (BSA) and with an Investigator Global Assessment (IGA) of disease severity of 2 or greater at baseline. Minimum body weight of 10 kg (22 lbs). History of atopic dermatitis and stable disease for at least 1 month according to the subject or caregiver.

Compound: RVT-501 0.5% ointment, applied twice daily for 28 days, Formulation C2 (see Table 1).

Criteria for Evaluation/Endpoints

Primary Endpoints: Frequency and severity of AE (local and systemic), Laboratory values, Vital signs, Plasma concentrations of RVT-501 and M11 metabolite.

Secondary Endpoints: Change from baseline in IGA at Week 4. Proportion of subjects with IGA score of 0 (clear) or 1 (almost clear) with at least a 2-point improvement from baseline at Week 4. Proportion of subjects with IGA score of 0 or 1 at Week 4. Percent change from baseline in Eczema Area and Severity Index (EASI) at Week 4. Proportion of subjects who achieved at least a 50% reduction from baseline EASI (EASI-50) at Week 4. Percent change from baseline in peak pruritus as measured with the Numeric Rating Scale (NRS) at Week 4. Percent change from baseline in BSA affected by disease at Week 4. Change from baseline in subject or caregiver assessment of itch severity. Change from baseline in subject or caregiver global assessment of change in itch severity.

Statistical Methods

Analysis Populations: All subjects enrolled in the study who had at least one application of study drug were included in the Safety Set. This was the population for the safety and efficacy analyses. The PK Set included all subjects who underwent plasma PK sampling and had evaluable PK assay results.

Safety Analyses: The number and proportion of subjects with AEs were summarized by system organ class, and preferred term for all AEs, all AEs considered by the investigator to be related to study drug, all serious adverse events (SAEs), and all AEs leading to study discontinuation.

Laboratory data were analyzed using descriptive summary statistics and changes from baseline. Incidence of treatment-emergent laboratory values that were considered clinically significantly abnormal were summarized. Vital sign data were listed by subject and summarized by treatment. Electrocardiogram data were listed.

No formal statistical comparisons were made for safety data.

Pharmacokinetic Analyses: RVT-501 and M11 were measured in plasma by a validated assay. The number and percent of subjects with measurable concentration at each time point and at any time during the study were summarized. RVT-501 and M11 concentrations were summarized descriptively at each collection time point.

Efficacy Analyses: Key efficacy endpoints included two-sided p-values based on one-sample t-tests for continuous endpoints. Observed cases were used for the primary analysis. The sensitivity analysis was based on the last observation carried forward (LOCF) for continuous data and non-responder imputation (NRI) for binary response data for missing data.

The IGA scores were summarized for the actual and change from baseline. The 90% confidence intervals (CIs) for the change from baseline were presented. IGA was also summarized as a categorical variable where n (%) of subjects were presented via a shift table. The IGA responder endpoint was defined as IGA score of 0 or 1 with at least a 2-point improvement from baseline at Week 4. The exact binomial 90% CIs were summarized. A similar analysis was presented for subjects who achieved an IGA score of 0 or 1 at Week 4.

The total EASI scores were summarized descriptively for actual, change from baseline, and percent change from baseline. The 90% CIs for the change and percentage change from baseline were presented. The proportion of subjects who achieved at least a 50% reduction from baseline total EASI (i.e., EASI 50) was presented with exact binomial 90% CIs.

The total affected BSA, in-office peak pruritus NRS, and weekly average peak pruritus NRS were summarized for the actual, change from baseline, and percent change from baseline. The 90% CIs for the change and percentage change from baseline were presented. The assessments of itch severity and global assessments of change in itch severity, as well as the subject reported symptoms and outcomes, were listed and summarized.

Interim Analyses: No interim analysis was performed for this study.

Summary of Results

Study Disposition: A total of 26 subjects were enrolled, and 25 completed the study. All subjects who entered the study (n=26) were included in the Safety Set. One subject was excluded from the PK Set (n=25); the subject missed the Week 1 visit because of two SAEs (asthma exacerbation and pneumonia) that were not related to study treatment, and did not complete the study.

Demographic and Baseline Characteristics: Subjects with atopic dermatitis had, on average, 43.5% of their body surface area covered with atopic dermatitis. Most subjects had an IGA 2 or 3 (mild or moderate severity) at baseline, and all were Black or African American or White.

Safety Results

Overall, 7 subjects (26.9%) experienced at least one AE after the first application of study drug, with a total of nine AEs reported. One subject (3.8%) had two SAEs (asthma exacerbation and pneumonia) that were of CTCAE grade 3 (severe), and deemed not related to the study treatment. All other AEs were of mild or moderate severity.

Only 1 subject (3.8%) experienced an AE that was judged by the investigator to be related to the study treatment (mild skin burning at the application site that was coded to application site pain). This treatment-related AE lasted about 2 days and did not lead to study discontinuation. No events of application site pruritus or stinging were reported. There were no clinically significant findings in safety clinical chemistry or hematology laboratory tests that resulted in an AE, and no trends detected for the safety laboratory results and vital signs. Only 1 subject (3.8%) had a clinically significant urinalysis value that was associated with a urinary tract infection, judged unrelated to the study treatment.

Pharmacokinetic Results

Ten subjects (40) had measurable concentrations of RVT-501 and of the M11 metabolite in plasma at one or more time points and 15 subjects (60%) had no measurable concentration at all time points. Only four subjects (16%) had concentrations of RVT-501 during the study that were ≥80 ng/ml (highest value: 1 860 ng/mL). One of these subjects also had a relatively high plasma concentration of the M11 metabolite (23.4 ng/mL). There were no trends in the demographics or baseline severity of atopic dermatitis of these subjects (aged 2 to 8 years, IGA of 2 or 3, 34% to 81% BSA, EASI between 5.8 and 30.5). See Table 40.

TABLE 40

| | RVT-501 0.5% | | | |
|---|---|---|---|---|
| | (N = 25) | | | |
| Time point Statistics (ng/mL) | RVT-501 | | M11 | |
| Week 1 Pre-Dose | | | | |
| N | 25 | | 25 | |
| Mean (SD) | 6.00 | (29.38) | 0.57 | (1.63) |
| Median | 0.0 | | 0.0 | |
| Min, Max | 0, 147.00 | | 0, 7.64 | |
| IQR | 0.0-0.0 | | 0.0-0.0 | |
| Number of subjects with | 6 | (24.0) | 6 | (24.0) |
| measurable concentrations, | | | | |
| n (%) | | | | |
| Week 1 Hour 3 | | | | |
| N | 25 | | 25 | |
| Mean (SD) | 102.92 | (392.58) | 0.61 | (1.75) |
| Median | 0.0 | | 0.0 | |
| Min, Max | 0, 1860.00 | | 0, 8.58 | |
| IQR | 0.0-0.0 | | 0.0-0.57 | |
| Number of subjects with | 6 | (24.0) | 7 | (28.0) |
| measurable concentrations, | | | | |
| n (%) | | | | |
| Week 1 Hour 7 | | | | |
| N | 25 | | 25 | |
| Mean (SD) | 62.16 | (293.74) | 1.24 | (4.67) |
| Median | 0.0 | | 0.0 | |
| Min, Max | 0, 1470.00 | | 0, 23.40 | |
| IQR | 0.0-0.47 | | 0.0-0.33 | |
| Number of subjects with | 8 | (32.0) | 7 | (28.0) |
| measurable concentrations, | | | | |
| n (%) | | | | |
| Week 4 Pre-Dose | | | | |
| N | 23 | | 23 | |
| Mean (SD) | 0.05 | (0.13) | 0.12 | (0.30) |
| Median | 0.0 | | 0.0 | |
| Min, Max | 0, 0.45 | | 0, 1.07 | |
| IQR | 0.0-0.0 | | 0.0-0.0 | |
| Number of subjects with | 3 | (13.0) | 4 | (17.4) |
| measurable concentrations, | | | | |
| n (%) | | | | |

Ten subjects (40%) had measurable concentrations of RVT-501 and of the M11 metabolite in plasma at one or more time points, and most of these had concentrations near the lower limit of quantitation (0.25 ng/mL).

At the Week 1 visit, the mean plasma concentration of RVT-501 was 6.00 ng/mL pre-dose, increased 3 hours post-dose to 102.92 ng/mL, and decreased 7 hours post-dose to 62.16 ng/mL. The mean plasma concentration before the study product application at the Week 4 visit was

ng/mL.

Four subjects (16%) had concentrations of RVT-501 ≥80 ng/ml measured at the Week 1 visit.

Subject 03001 was 3 years old, had an IGA of 3, an EASI of 24.7, and a BSA of 71.9% at the baseline visit. The plasma level of RVT-501 was 80.3 ng/mL 7 hours post-dose.

Subject 03002 was 8 years old, had an IGA of 2, an EASI of 8.7, and a BSA of 48.8% at the baseline visit. The plasma level of RVT-501 was 710.0 ng/mL 3 hours post-dose.

Subject 03005 was 7 years old, had an IGA of 2, an EASI of 5.8, and a BSA of 34.0% at the baseline visit. The plasma level of RVT-501 was 1 860.0 ng/mL 3 hours post-dose.

Subject 03007 was 2 years old, had an IGA of 3, an EASI of 30.5, and a BSA of 81.0% at the baseline visit. The plasma level of RVT-501 was 147.0 ng/mL and 1 470.0 ng/mL pre-dose and 7 hours post-dose, respectively.

There were no deviations related to study drug application at these visits or to PK sampling time reported for these subjects.

The mean plasma concentration of the M11 metabolite was below 1 ng/ml at all time points with the exception of a mean value of 1.24 ng/ml at 7 hours post-dose at the Week 1 visit. The highest concentration measured was 23.40 ng/mL in Subject 03007 observed at 7 hours post-dose at the Week 1 visit.

Efficacy Results (see Table 41): The Safety Set was the primary population used for the efficacy analyses. A total of 30.8% of the subjects achieved an IGA of clear or almost clear with at least a 2-point improvement from baseline after 4 weeks of treatment. A total of 46.2% of the subjects achieved an IGA of clear or almost clear at Week 4 visit. A reduction of at least 50% in EASI was observed in 61.5% of the subjects after 4 weeks of treatment. Statistically significant percent reductions from baseline were also observed in EASI, total affected BSA, and pruritus at Week 4 (table below).

Subjects aged 7 to 11 years old had a numerically better response to RVT-501 0.5% ointment than subjects aged 2 to 6 years old for all endpoints assessed. Five subjects (38.5%) in this age subgroup achieved at least a 2-point improvement in IGA to clear or almost clear at Week 4 compared to 3 subjects (23.1%) in the 2 to 6 age subgroup.

TABLE 41
Results

| | | |
|---|---|---|
| | RVT-501 0.5% | P value for difference |
| Statistics | (N = 26) | between the groups |

Proportion of Subjects with at Least a 2-PointImprovement in IGA
to Clear or Almost Clear at Week 4

| | | | |
|---|---|---|---|
| N(%) | 8 | (30.8) | N/A |

Proportion of Subjects with an IGA of Clear orAlmost Clear at Week 4

| | | | |
|---|---|---|---|
| N(%) | 12 | (46.2) | N/A |

Proportion of Subjects who Achieved EASI-50 at Week4

| | | | |
|---|---|---|---|
| N(%) | 16 | (61.5) | N/A |

Percent Change from Baseline in EASI at Week4

| | | | |
|---|---|---|---|
| Mean (SD) | −64.87 | (30.802) | <0.001 |

Percent Change from Baseline in Total Affected BSAat Week 4

| | | | |
|---|---|---|---|
| Mean(SD) | −54.15 | (37.871) | <0.001 |

Percent Change from Baseline in Peak Pruritus NRSScore at Week 4

| | | | |
|---|---|---|---|
| Mean(SD) | −56.52 | (33.853) | <0.001 |

BSA = body surface area;
EASI = eczema area and severity index;
IGA = investigator global assessment;
NRS = numeric rating scale;
SD = standard deviation.

The proportion of subjects who achieved an IGA of clear or almost clear with at least a 2-point improvement from baseline and achieved an IGA of clear or almost clear is presented in Table 42 for the Safety Set. A proportion of 30.8% of the subjects were responders, defined as achieving an IGA of clear or almost clear with at least a 2-point improvement from baseline after 4 weeks of treatment with RVT-501 0.5%. Only 2 subjects (8.0%) achieved this endpoint at Week 1.

Table 42 also presents the proportion of subjects who achieved an IGA of clear or almost clear in the Safety Set. A proportion of 46.2% of the subjects achieved an IGA of clear or almost clear after 4 weeks of treatment with RVT-501 0.5%. Only 2 subjects (8.0%) achieved this endpoint at Week 1.

TABLE 42
IGA Responder Analyses (Safety Set)

| | At least 2-point | |
|---|---|---|
| | improvement to | Improvement to clear |
| | clear or almost clear | or almost clear |
| Time point Response | (N = 26) | (N = 26) |
| Week 1 | | |
| Subjects with non- | 25 | 25 |
| missing IGA, n | | |
| Subjects achieving | 2 (8.0) | 2 (8.0) |
| the endpoint, n (%) | | |
| 90% CI | 1.4, 23.1 | 1.4, 23.1 |
| Week 4 | | |
| Subjects with non- | 26 | 26 |
| missing IGA, n | | |
| Subjects achieving | 8 (30.8) | 12 (46.2) |
| the endpoint, n (%) | | |
| 90% CI | 16.3, 48.7 | 29.2, 63.8 |

A summary of IGA scores over time, including change from baseline, is provided in Table 43 for the Safety Set. There was a constant decrease in IGA score over time with a mean decrease of approximately 1.0 point in IGA score after 4 weeks of treatment with RVT-501 0.5% ointment.

TABLE 43
Summary of IGA Scores Over Time (Safety Set)

| Time Point | RVT-501 0.5% | |
|---|---|---|
| Parameter Statistics | (N = 26) | |
| Baseline | | |
| N | 26 | |
| Mean (SD) | 2.7 | (0.56) |
| Median | 3.0 | |
| Min, Max | 2, 4 | |
| IQR | 2.0-3.0 | |
| Week 1 | | |
| N | 25 | |
| Mean (SD) | 2.1 | (0.53) |
| Median | 2.0 | |
| Min, Max | 1, 3 | |
| IQR | 2.0-2.0 | |
| Mean change from baseline (90% CI) | −0.6 | (−0.8, −0.3) |
| Week 4 | | |
| N | 26 | |
| Mean (SD) | 1.6 | (0.94) |
| Median | 2.0 | |
| Min, Max | 0, 3 | |
| IQR | 1.0-2.0 | |
| Mean change from baseline (90% CI) | −1.0 | (−1.4, −0.7) |

Eczema Area and Severity Index: The proportion of subjects who achieved at least a 50% reduction from baseline in the total EASI score (EASI-50) at Week 4 is presented in Table 44 for the Safety Set. A proportion of 61.5% of the subjects achieved an EASI-50 after 4 weeks of treatment with RVT-501 0.5%. Eight subjects (32.0%) achieved this endpoint at Week 1.

TABLE 44
Proportion of Subjects Who Achieved EASI-50 Safety Set)

| Time Point | At least 50% Reduction |
|---|---|
| Response | (N = 26) |
| Week 1 | |
| Subjects with non-missing EASI, n | 25 |
| Subjects who achieved EASI-50, n (%) | 8 (32.0) |
| 90% CI | 17.03, 50.36 |
| Week 4 | |
| Subjects with non-missing EASI, n | 26 |
| Subjects who achieved EASI-50, n (%) | 16 (61.5) |
| 90% CI | 43.57, 77.43 |

A summary of EASI scores over time, including change and percent change from baseline, is provided in Table 45 for the Safety Set. There was a constant decrease in EASI score over time with a mean reduction of 64.9% after 4 weeks of treatment with RVT-501 0.5% ointment. The percent change from baseline at Week 4 was statistically significant (P<0.001).

TABLE 45
Summary of EASI Scores Over Time (Safety Set)

| Time Point | RVT-501 0.5% | |
|---|---|---|
| Parameter Statistics | (N = 26) | |
| Baseline | | |
| N | 26 | |
| Mean (SD) | 18.63 | (11.481) |
| Median | 17.55 | |
| Min, Max | 4.5, 55.4 | |
| IQR | 9.40-24.80 | |
| Week 1 | | |
| N | 25 | |
| Mean (SD) | 12.30 | (9.457) |
| Median | 9.20 | |
| Min, Max | 2.8, 44.0 | |
| IQR | 6.00-15.80 | |
| Mean change from baseline (90% CI) | −6.81 | (−8.92, −4.70) |
| Mean percent change from baseline | −32.8 | (−41.9, −23.8) |
| (90% CI) | | |
| Week 4 | | |
| N | 26 | |
| Mean (SD) | 6.52 | (6.653) |
| Median | 3.90 | |
| Min, Max | 0.0, 20.3 | |
| IQR | 0.90-10.20 | |
| Mean change from baseline (90% CI) | −12.12 | (−15.03, −9.20) |
| Mean percent change from baseline | −64.9 | (−75.2, −54.6) |
| (90% CI) | | |

| | |
|---|---|
| P value¹ | <0.001 |

Body Surface Area: A summary of total affected BSA over time, including change and percent change from baseline, is provided in Table 46 for the Safety Set. There was a constant decrease in affected BSA over time with a mean reduction of 54.2% after 4 weeks of treatment with RVT-501 0.5% ointment. The percent change from baseline at Week 4 was statistically significant (P<0.001).

TABLE 46
Summary of BSA Over Time (Safety Set)

| Time Point | RVT-501 0.5% | |
|---|---|---|
| Parameter Statistics | (N = 26) | |
| Baseline | | |
| N | 26 | |
| Mean (SD) | 43.53 | (17.854) |
| Median | 40.00 | |
| Min, Max | 26.2, 88.6 | |
| IQR | 28.20-52.60 | |
| Week 1 | | |
| N | 25 | |
| Mean (SD) | 35.97 | (20.759) |
| Median | 26.30 | |
| Min, Max | 15.0, 86.0 | |
| IQR | 21.80-46.50 | |
| Mean change from baseline (90% CI) | −8.05 | (−11.181, −4.915) |
| Mean percent change from baseline | −20.3 | (−27.87, −12.76) |
| (90% CI) | | |
| Week 4 | | |
| N | 26 | |
| Mean (SD) | 22.69 | (24.817) |
| Median | 9.50 | |
| Min, Max | 0.0, 76.3 | |
| IQR | 4.90-41.80 | |
| Mean change from baseline (90% CI) | −20.84 | (−26.08, −15.60) |
| Mean percent change from baseline | −54.2 | (−66.8, −41.5) |
| (90% CI) | | |

| | |
|---|---|
| P value¹ | <0.001 |

Weekly Average Peak Pruritus Numeric Rating Scale: A summary of weekly average peak pruritus NRS over time, including change and percent change from baseline, is provided in Table 47 for the Safety Set. There was a constant decrease in the weekly average peak pruritus over time with a mean reduction of 56.5% after 4 weeks of treatment with RVT-501 0.5% ointment. The percent change from baseline at Week 4 was statistically significant (P<0.001).

TABLE 47
Summary of Weekly Average Peak Pruritus
NRS Over Time (Safety Set)

| Time Point | RVT-501 0.5% | |
|---|---|---|
| Parameter Statistics | (N = 26) | |
| Baseline | | |
| N | 26 | |
| Mean (SD) | 6.56 | (2.408) |
| Median | 6.79 | |
| Min, Max | 2.0, 10.0 | |
| IQR | 4.43-8.86 | |
| Week 1 | | |
| N | 24 | |
| Mean (SD) | 4.20 | (2.818) |
| Median | 3.46 | |
| Min, Max | 0.0, 9.0 | |
| IQR | 2.00-6.57 | |
| Mean change from baseline (90% CI) | −2.50 | (−3.310, −1.692) |
| Mean percent change from baseline | −38.3 | (−51.16, −25.37) |
| (90% CI) | | |
| Week 4 | | |
| N | 25 | |
| Mean (SD) | 3.01 | (2.815) |
| Median | 2.83 | |
| Min, Max | 0.0, 9.0 | |
| IQR | 0.43-4.29 | |
| Mean change from baseline (90% CI) | −3.62 | (−4.480, −2.753) |
| Mean percent change from baseline | −56.5 | (−68.10, −44.94) |
| (90% CI) | | |

| | |
|---|---|
| P value¹ | <0.001 |

In-Office Peak Pruritus Numeric Rating Scale Over Time: A summary of in-office peak pruritus NRS over time, including change and percent change from baseline, is provided in Table 48 for the Safety Set. There was a constant decrease in the in-office peak pruritus over time with a mean reduction of 47.6% after 4 weeks of treatment with RVT-501 0.5% ointment.

TABLE 48
Summary of In-Office Peak Pruritus NRS Over Time (Safety Set)

| Time Point | RVT-501 0.5% | |
|---|---|---|
| Parameter Statistics | (N = 26) | |
| Baseline | | |
| N | 26 | |
| Mean (SD) | 7.2 | (2.16) |
| Median | 8.0 | |
| Min, Max | 4, 10 | |
| IQR | 5.0-9.0 | |
| Week 1 | | |
| N | 25 | |
| Mean (SD) | 4.3 | (3.10) |
| Median | 3.0 | |
| Min, Max | 0, 9 | |
| IQR | 2.0-8.0 | |
| Mean change from baseline (90% CI) | −3.1 | (−4.22, −1.94) |
| Mean percent change from baseline | −39.2 | (−54.45, −23.96) |
| (90% CI) | | |
| Week 4 | | |
| N | 25 | |
| Mean (SD) | 3.8 | (2.93) |
| Median | 4.0 | |
| Min, Max | 0, 10 | |
| IQR | 1.0-6.0 | |
| Mean change from baseline (90% CI) | −3.6 | (−4.66, −2.54) |
| Mean percent change from baseline | −47.6 | (−61.85, −33.35) |
| (90% CI) | | |

Discussion

The objectives of this study were to evaluate the safety and PK of topical RVT-501 in pediatric subjects aged 2 to 11 years old with atopic dermatitis, under maximal use conditions. The efficacy of the drug in this population was also assessed as a secondary objective.

The trial enrolled subjects with atopic dermatitis who generally have a more extensive form of disease. Subjects with atopic dermatitis had, on average, 43.5% of their body surface area covered with atopic dermatitis. Most subjects had an IGA 2 or 3 (mild or moderate severity) at baseline. The study had an equal distribution across both age groups (ages 2 to 6 and ages 7 to 11).

RVT-501 0.5% ointment was well tolerated in subjects with extensive atopic dermatitis. One subject experienced two SAEs that were deemed not related to the study drug by the investigator. All AEs, but one, were considered unrelated to the study drug. One subject (3.8%) reported a mild skin burning sensation at the application site that was judged to be related to the study drug and lasted about 2 days. No events of application site pruritus or stinging were reported. There were no clinically significant findings for clinical chemistry and hematology laboratory tests, or vital signs, and 1 subject (3.8%) had a clinically significant urinalysis result associated with a urinary tract infection, judged unrelated to the study treatment.

Consistent with other studies, no or minimal systemic absorption was observed for the majority of subjects following topical application of RVT-501 0.5% ointment. Ten subjects (40%) had measurable concentrations of RVT-501 and of the M11 metabolite in plasma at one or more time points and 15 subjects (60%) had no measurable concentration at all time points. Only four subjects (16%) had concentrations of RVT-501 during the study that were ≥80 ng/ml (highest value: 1 860 ng/mL). One of these subjects also had a relatively high plasma concentration of the M11 metabolite (23.4 ng/mL). There were no trends in the demographics or baseline severity of atopic dermatitis of these subjects (aged 2 to 8 years, IGA of 2 or 3, 34% to 81% BSA, EASI between 5.8 and 30.5). After 4 weeks of twice daily applications, 30.8% of the subjects achieved an IGA of clear or almost clear with at least a 2-point reduction from baseline.

Evaluation of the other efficacy endpoints also suggests a positive effect of RVT-501 on atopic dermatitis for this pediatric population. A total of 46.2% of the subjects achieved an IGA of clear or almost clear, 61.5% achieved a reduction of at least 50% in EASI, and statistically significant percent reductions from baseline were observed in EASI, total affected BSA, and pruritus.

Conclusions for the potential for efficacy are, however, limited due to the absence of a vehicle control.

Conclusions: RVT-501 0.5% ointment was generally safe and well tolerated in pediatric subjects with extensive atopic dermatitis. Two SAEs, both evaluated as unrelated to the study treatment, were observed in the same subject. Only one subject reported an application site burning sensation, and no subjects reported application site pruritus or stinging.

There were no significant or clinically meaningful changes in clinical chemistry and hematology laboratory tests, or vital signs.

Ten subjects (40%) had measurable concentrations of RVT-501 and of the M11 metabolite in plasma at one or more time points, and most of these had concentrations near the lower limit of quantitation. Four subjects (16%) had concentrations of RVT-501 in plasma at one or more time point that were ≥80 ng/ml (highest value: 1 860 ng/mL).

RVT-501 0.5% was associated with improvements in atopic dermatitis as seen by the reductions in IGA, EASI, BSA, and pruritus assessments.

1. A method of treating a skin condition in a patient in need thereof comprising topically applying a topical composition comprising a therapeutically effective amount of methyl N-[3-(6,7-dimethoxy-2-methylaminoquinazolin-4-yl)phenyl]terephthalamic acid, PEG 400, PEG 4000, white petrolatum, vitamin E, glycerol monostearate/glycerides, isopropyl myristate, and water.

2. The method of claim 1, wherein methyl N-[3-(6,7-dimethoxy-2-methylaminoquinazolin-4-yl)phenyl]terephthalamic acid is at a concentration of about 0.01% to about 5% by weight of the topical composition.

3. The method of claim 1, wherein PEG 400 is at a concentration of about 25% to about 75% by weight of the topical composition.

4. The method of claim 1, wherein PEG 4000 is at a concentration of about 15% to about 35% by weight of the topical composition.

5. The method of claim 1, wherein white petrolatum is at a concentration of about 1% to about 10% by weight of the topical composition.

6. The method of claim 1, wherein vitamin E is at a concentration of about 0.01% to about 5% by weight of the topical composition.

7. The method of claim 1, wherein glycerol monostearate/glycerides is at a concentration of about 2% to about 15% by weight of the topical composition.

8. The method of claim 1, wherein isopropyl myristate is at a concentration of about 2% to about 25% by weight of the topical composition.

9. The method of claim 1, wherein water is at a concentration of about 0.1% to about 10% by weight of the topical composition.

10. The method of claim 1, wherein methyl N-[3-(6,7-dimethoxy-2-methylaminoquinazolin-4-yl)phenyl]terephthalamic acid is at 0.2% by weight, PEG 400 is at 50.5% by weight, PEG 4000 is at 25.0% by weight, white petrolatum is at 4.4% by weight, vitamin E is at 0.1% by weight, glycerol monostearate/glycerides is at 8.0% by weight, isopropyl myristate is at 10.0% by weight, and water is at 2.0% by weight.

11. The method of claim 1, wherein methyl N-[3-(6,7-dimethoxy-2-methylaminoquinazolin-4-yl)phenyl]terephthalamic acid is at 0.5% by weight, PEG 400 is at 50.5% by weight, PEG 4000 is at 25.0% by weight, white petrolatum is at 4.4% by weight, vitamin E is at 0.1% by weight, glycerol monostearate/glycerides is at 8.0% by weight, isopropyl myristate is at 10.0% by weight, and water is at 2.0% by weight.

12. The method of claim 1, wherein the skin condition is selected from the group consisting of dermatitis; psoriasis; itchy skin; acne; inflammation and redness of the skin; disorders associated with sebaceous glands; oily skin; dry skin; rosacea; burns; disorders affecting the palms or soles; genetic disorders of the skin; warts; and any combination thereof.

13. The method of claim 12, wherein dermatitis is selected from the group consisting of atopic dermatitis, contact dermatitis, allergic contact dermatitis, irritant contact dermatitis, stasis dermatitis, seborrheic dermatitis, chronic dermatitis, eczema, and any combination thereof.

14. The method of claim 12, wherein psoriasis is selected from the group consisting of plaque psoriasis, guttate psoriasis, inverse psoriasis, pustular psoriasis, erythrodermic psoriasis, and any combination thereof.

15. The method of claim 12, wherein itchy skin is selected from the group consisting of pruritus, prurigo, Pityriasis rubra pilaris, lichen simplex chronicus, lichen planus, and any combination thereof.

16. The method of claim 12, wherein acne is selected from the group consisting of acne vulgaris, cystic acne, inflammatory acne, non-inflammatory acne, and any combination thereof.

17. The method of claim 12, wherein inflammation and redness of the skin is selected from the group consisting of seborrheic dermatitis, urticaria eczema, hives, seborrheic eczema, and any combination thereof.

18. The method of claim 12, wherein disorders associated with sebaceous glands is selected from the group consisting of acne, follicular hyperkeratinization, sebostasis, sebaceous adenomas, sebaceous hyperplasia, excess sebum production, seborrhea, sebaceoma, sebaceous carcinoma, seborrheic dermatitis, sebaceous cysts, and any combination thereof.

19. The method of claim 12, wherein oily skin is seborrhea.

20. The method of claim 12, wherein dry skin is selected from the group consisting of sebostasis, ichthyosis, xerosis, and any combination thereof.

21. The method of claim 12, wherein burns is sunburn.

22. The method of claim 12, wherein disorders affecting the palms or soles is selected from the group consisting of palmoplantar pustulosis, exfoliative keratolysis, and any combination thereof.

23. The method of claim 12, wherein genetic disorders of the skin is Darier's disease.

24. The method of claim 1, wherein said patient is an adolescent.

25. The method of claim 1, wherein said skin condition is atopic dermatitis.
